(19) United States
(12) Patent Application Publication
(10) Pub. No.: US 2012/0231023 A1
(43) Pub. Date: Sep. 13, 2012

(54) Novel Vaccine Adjuvants Based on Targeting Adjuvants to Antibodies Directly to Antigen-Presenting Cells

(75) Inventors: Gerard Zurawski, Midlothian, TX (US); Jacques F. Banchereau, Montclair, NJ (US); Anne-Laure Flamar, Dallas, TX (US); Robert R. Kane, Waco, TX (US)

(73) Assignee: BAYLOR RESEARCH INSTITUTE, Dallas, TX (US)

(21) Appl. No.: 13/415,564

(22) Filed: Mar. 8, 2012

Related U.S. Application Data
(60) Provisional application No. 61/450,480, filed on Mar. 8, 2011.

(51) Int. Cl. A61K 39/44; A61K 35/12; A61P 31/12; C12N 5/0784; A61P 31/10; A61P 33/00; A61P 33/02; A61P 37/08; A61P 35/00; A61P 31/04

(52) U.S. Cl. 424/178.1; 424/93.7; 435/375

(57) ABSTRACT

The present invention includes compositions and methods for enhancing an immune response with an adjuvant composition comprising: an anti-dendritic cell (DC)-specific antibody or fragment thereof conjugated to at least a portion of Toll-Like Receptor (TLR) agonist, more specifically a TLR7 ligand (TLR7L), and a pharmaceutically acceptable carrier, wherein the conjugate and agonist are each comprised in an amount such that, in combination with the other, are effective to produce the immune response in a human or animal subject in need of immunostimulation.

### CROSS-REFERENCE TO RELATED APPLICATIONS

The present application claims the benefit of priority to U.S. Provisional Patent Application No. 61/450,480 filed Mar. 8, 2011, the entire contents of which are incorporated herein.

### STATEMENT OF FEDERALLY FUNDED RESEARCH

This invention was made with U.S. Government support under Contract Nos. 1 U19 AI057234 awarded by the National Institutes of Health (NIH)/National Institute for Allergy and Infectious Diseases (NIAID). The government has certain rights in this invention.

### TECHNICAL FIELD OF THE INVENTION

The present invention relates in general to the dendritic cell (DC)-targeting vaccines, and more particularly, to the enhancing vaccine efficacy by directly linking adjuvants (e.g., TLR ligands/agonists) directly to DC-targeting vaccines.

### REFERENCE TO A SEQUENCE LISTING

The instant application contains a Sequence Listing which has been submitted in ASCII format via EFS-Web and is hereby incorporated by reference in its entirety.

### BACKGROUND OF THE INVENTION

Without limiting the scope of the invention, its background is described in connection with novel adjuvants, dendritic cell (DC) vaccines, and strategies for enhancing immune responses to DC vaccines.

U.S. Patent Application No. 20090004194 (Kedl, 2007) relates to novel protein and DNA conjugates which promote antigen specific cellular immunity. The use of these polypeptide conjugates and DNA conjugates as immune adjuvants for treating various chronic diseases including cancer, infectious diseases, autoimmune diseases, allergic and inflammatory diseases. The Kedl invention discloses fusion proteins and DNA conjugates containing a TLR/CD40/agonist and optional antigen combination. The use of these protein and DNA conjugates as immune adjuvants and as vaccines for treatment of various chronic diseases is also taught.

U.S. Patent Application No. 20080220011 (Steven, 2008) provides a fusion protein comprising a flagellin adjuvant and a tumor antigen. Also provided are compositions comprising a flagellin adjuvant and a tumor antigen. The invention further provides pharmaceutical formulations and methods for inducing an immune response against a tumor antigen and methods of treating a tumor in a subject.

U.S. Patent Application No. 20080248068 (Ljunggren et al., 2008) is directed to flagellin and its use as an adjuvant for vaccination. The invention can be used in vaccine formulations to improve immunity against any other antigen administered at the same localization. The antigen can be administered in the same construct as Flagellin or in any other formulation given at the same localization. As an alternative flagellin can be used to stimulate immunity against antigens expressed at a specific location. Flagellin can also be used to induce local inflammation with the purpose of creating a model for inflammation.

U.S. Pat. No. 7,404,963 issued to Sotomayor and Suarez (2008) provides adjuvants, vaccines, and related methods that are useful in eliciting immune responses, particularly immune responses against tumor antigens. According to the Sotomayor invention flagellin is capable of inhibiting tolerance when it is administered in conjunction with a tolerogenic antigen. This effect is likely mediated by the ability of flagellin to induce IL-12 while keeping IL-10 levels low. Furthermore, flagellin can be provided in an extended-releasing manner by using a flagellin-expressing cell. Preferably, the flagellin-expressing cell is treated such that it is no longer capable of replicating, yet retaining the ability to express flagellin, such as by lethal irradiation.

### SUMMARY OF THE INVENTION

The present invention describes compositions and methods for making novel vaccine adjuvants based on targeting adjuvants with antibodies directly to antigen-presenting cells. The present invention was found to enhance vaccine efficacy by directly linking adjuvants (e.g., TLR ligands) directly to DC-targeting vaccines. As shown herein, the compositions and methods of the present invention are broadly applicable to all DC-targeting vaccines and extensible to making adjuvants with unexpected novel properties.

An adjuvant composition is disclosed in one embodiment of the present invention. The adjuvant comprises an anti-dendritic cell (DC)-specific antibody or fragment thereof conjugated to a Toll-Like Receptor (TLR) agonist; and a pharmaceutically acceptable carrier, wherein the conjugate and agonist are each comprised in an amount such that, in combination with the other, are effective to produce the immune response in a human or animal subject in need of immunostimulation. The DC-specific antibody or fragment as disclosed herein is selected from an anti-DCIR, MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, or ASPGR. In a related aspect a nucleotide sequence of the DC-specific antibody is selected from SEQ ID NOS.: 3 to 67 and an amino acid sequence of the DC-specific antibody is selected from SEQ ID NOS.: 68 to 132.

The composition of the present invention further comprises antigenic peptides selected from human immunodeficiency virus (HIV) antigens and gene products selected from the group consisting of gag, pol, and env genes, the Nef protein, reverse transcriptase, string of HIV peptides (Hipo5), PSA (KLQCVDLHV)-tetramer (SEQ ID NO: 159), a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide (GILGFVFTL) tetramer (SEQ ID NO: 160), and Avian Flu (HA5-1), dockerin domain from C. thermocellum, measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens or combinations and modifications thereof. In one aspect the composition further comprises antigenic peptides selected from cancer peptides are selected from tumor associated antigens comprising antigens from leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia. In another aspect the composition further comprises antigenic peptides selected from tumor associated antigens are selected from CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67. In yet another aspect the DC-specific antibody is humanized. In another aspect the composition is administered to the human or animal subject by an oral route, a nasal route, topically or as an injection, wherein the injection is selected from the group consisting of subcutaneous, intravenous, intraperitoneal, intramuscular, and intravenous.

Another embodiment of the present invention relates to a vaccine composition comprising: an antigen; and an adjuvant, wherein the adjuvant comprises an anti-dendritic cell (DC)-specific antibody or fragment thereof conjugated to a Toll-Like Receptor (TLR) agonist; and a pharmaceutically acceptable carrier, wherein the conjugate and agonist are each comprised in an amount such that, in combination with the other, are effective to produce the immune response in a human or animal subject in need of immunostimulation. In one aspect the DC-specific antibody or fragment is selected from an anti-DCIR, MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, or ASPGR. In another aspect the composition further comprises antigenic peptides selected from human immunodeficiency virus (HIV) antigens and gene products selected from the group consisting of gag, pol, and env genes, the Nef protein, reverse transcriptase, string of HIV peptides (Hipo5), PSA (KLQCVDLHV)-tetramer (SEQ ID NO: 159), a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide (GILGFVFTL) tetramer (SEQ ID NO: 160), and Avian Flu (HA5-1), dockerin domain from C. thermocellum, measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens or combinations and modifications thereof. In yet another aspect the composition further comprises antigenic peptides selected from cancer peptides are selected from tumor associated antigens comprising antigens from leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia. In another aspect the composition further comprises antigenic peptides selected from tumor associated antigens are selected from CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67.

In other related aspects the DC-specific antibody is humanized and the composition is administered to the human or animal subject by an oral route, a nasal route, topically or as an injection, wherein the injection is selected from the group consisting of subcutaneous, intravenous, intraperitoneal, intramuscular, and intravenous.

In yet another embodiment the present invention provides a method for increasing effectiveness of antigen presentation by an antigen presenting cell comprising the step of contacting the antigen presenting cell with a composition comprising an antigen and an adjuvant, wherein the adjuvant comprises an anti-dendritic cell (DC)-specific antibody or fragment thereof conjugated to a Toll-Like Receptor (TLR) agonist; and a pharmaceutically acceptable carrier, wherein the conjugate and agonist are each comprised in an amount such that, in combination with the other, are effective to produce the immune response in a human or animal subject in need of immunostimulation. In one aspect of the method the DC-specific antibody or fragment is selected from an anti-DCIR, MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, or ASPGR.

In another aspect the composition further comprises antigenic peptides selected from human immunodeficiency virus (HIV) antigens and gene products selected from the group consisting of gag, pol, and env genes, the Nef protein, reverse transcriptase, string of HIV peptides (Hipo5), PSA (KLQCVDLHV)-tetramer (SEQ ID NO: 159), a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide (GILGFVFTL) tetramer (SEQ ID NO: 160), and Avian Flu (HA5-1), dockerin domain from C. thermocellum, measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens or combinations and modifications thereof. In yet another aspect the composition further comprises antigenic peptides selected from cancer peptides are selected from tumor associated antigens comprising antigens from leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia. In another aspect the composition further comprises antigenic peptides selected from tumor associated antigens are selected from CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67. In a specific aspect of the method the DC-specific antibody is humanized and the composition is administered to the human or animal subject by an oral route, a nasal route, topically or as an injection. The type of injection employed in the method described hereinabove is selected from the group consisting of subcutaneous, intravenous, intraperitoneal, intramuscular, and intravenous.

One embodiment of the instant invention discloses a method for a treatment, a prophylaxis or a combination thereof against one or more cancers in a human subject comprising the steps of: (i) identifying the human subject in need of the treatment, the prophylaxis or a combination thereof and (ii) administering a vaccine composition comprising: an antigen; and an adjuvant, wherein the adjuvant comprises an anti-dendritic cell (DC)-specific antibody or fragment thereof conjugated to a Toll-Like Receptor (TLR) agonist; and a pharmaceutically acceptable carrier, wherein the conjugate and agonist are each comprised in an amount such that, in combination with the other, are effective to produce the immune response in a human or animal subject in need of immunostimulation.

In one aspect of the treatment method disclosed hereinabove the DC-specific antibody or fragment is selected from an anti-DCIR, MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, or ASPGR. In another aspect the composition further comprises antigenic peptides selected from human immunodeficiency virus (HIV) antigens and gene products selected from the group consisting of gag, pol, and env genes, the Nef protein, reverse transcriptase, string of HIV peptides (Hipo5), PSA (KLQCVDLHV)-tetramer (SEQ ID NO: 159), a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide (GILGFVFTL) tetramer (SEQ ID NO: 160), and Avian Flu (HA5-1), dockerin domain from C. thermocellum, measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens or combinations and modifications thereof. IN yet another aspect the composition further comprises antigenic peptides selected from cancer peptides are selected from tumor associated antigens comprising antigens from leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia. In another aspect the composition further comprises antigenic peptides selected from tumor associated antigens are selected from CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67.

In related aspects of the treatment method the DC-specific antibody is humanized and the composition is administered to the human or animal subject by an oral route, a nasal route, topically or as an injection (subcutaneous, intravenous, intraperitoneal, intramuscular or intravenous).

In another embodiment the instant invention describes a method for providing immunostimulation by activation of one or more dendritic cells (DCs) to a human subject for a prophylaxis, a therapy or a combination thereof against one or more viral, bacterial, fungal, parasitic, protozoal, and parasitic diseases, and allergic disorders comprising the steps of: (i) identifying the human subject in need of immunostimulation for the prophylaxis, the therapy or a combination thereof against the one or more diseases selected from the group selected from influenza, HIV, cancer, and immune disorders;

isolating one or more DCs from the human subject, (ii) activating the isolated DCs with an amount of a composition effective for form activated DCs comprising: (a) an antigen and (b) an adjuvant, wherein the adjuvant comprises an anti-dendritic cell (DC)-specific antibody or fragment thereof conjugated to a Toll-Like Receptor (TLR) agonist; and a pharmaceutically acceptable carrier, wherein the conjugate and agonist are each comprised in an amount such that, in combination with the other, are effective to produce the immune response in a human or animal subject in need of immunostimulation, and (iii) reintroducing the activated DCs into the human subject.

In one aspect of the method disclosed hereinabove the DC-specific antibody or fragment is selected from an anti-DCIR, MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, or ASPGR. In another aspect the composition further comprises antigenic peptides selected from human immunodeficiency virus (HIV) antigens and gene products selected from the group consisting of gag, pol, and env genes, the Nef protein, reverse transcriptase, string of HIV peptides (Hipo5), PSA (KLQCVDLHV)-tetramer (SEQ ID NO: 159), a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide (GILGFVFTL) tetramer (SEQ ID NO: 160), and Avian Flu (HA5-1), dockerin domain from C. thermocellum, measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens or combinations and modifications thereof. In yet another aspect the composition further comprises antigenic peptides selected from cancer peptides are selected from tumor associated antigens comprising antigens from leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia. In another aspect the composition further comprises antigenic peptides selected from tumor associated antigens are selected from CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67.

In one aspect of the method the DC-specific antibody is humanized. In another aspect the composition is administered to the human or animal subject by an oral route, a nasal route, topically or as an injection. In yet another aspect the injection is selected from the group consisting of subcutaneous, intravenous, intraperitoneal, intramuscular, and intravenous.

### BRIEF DESCRIPTION OF THE DRAWINGS

For a more complete understanding of the features and advantages of the present invention, reference is now made to the detailed description of the invention along with the accompanying figures and in which:

FIGS. 1A-1C show the chemical structures of TLR7L variants as described in the instant invention;

FIGS. 2A-2C show the dose titration of the TLR9, TLRf, and TLRf′ variants of the instant invention at concentrations of 10 nM, 100 nM, 1 μM, and 10 μM. The activity is measured by the secretion (pg/mL) of: IL-6 (FIG. 2A), IL-8 (FIG. 2B), IL-1β (FIG. 2C). The dose titration curves indicate that the activity of the 3 different TLR7-L compounds is pretty similar. All 3 compounds are still effective at 100 nM;

FIGS. 2D-2G show the dose titration of the TLR9, TLRf, and TLRf variants of the instant invention at concentrations of 12.5 nM, 25 nM, 50 nM, and 100 nM. The activity is measured by the secretion of: IL-6 (FIG. 2D), IL-8 (FIG. 2E), IL-1β (FIG. 2F), and IL-12p40 (FIG. 2G). The dose titration curves indicate that the TLRf is marginally more effective and its activity is still detected at 25 nM;

FIGS. 3A and 3B show that TLR7L linked to DC-targeting mAb potentiates and restricts adjuvant activity. The activity of the TLR7L linked to CohFluM1 is dependent on the targeting antibody. TLR7L linked to CohFluM1 or CohFluM1 alone are effective only at highest 30 nM dose. Moreover, there is a difference in potency and relative amounts of cytokines induced with anti-DCIR and anti-CD40 antibodies linked to TLR7L; and

FIG. 3C is a chemical structure of a TLR7 ligand according to an embodiment of the present invention.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an,” and “the” are not intended to refer to only a singular entity, but include the general class of which a specific example may be used for illustration. The terminology herein is used to describe specific embodiments of the invention, but their usage does not delimit the invention, except as outlined in the claims.

As used herein the term “Antigen presenting cells” (APC) are cells that are capable of activating T cells, and include, but are not limited to, certain macrophages, B cells and dendritic cells. “Dendritic cells” (DCs) refers to any member of a diverse population of morphologically similar cell types found in lymphoid or non-lymphoid tissues. These cells are characterized by their distinctive morphology, high levels of surface MHC-class II expression (Steinman, et al., Ann. Rev. Immunol. 9:271 (1991); incorporated herein by reference for its description of such cells). These cells can be isolated from a number of tissue sources, and conveniently, from peripheral blood, as described herein. Dendritic cell binding proteins refers to any protein for which receptors are expressed on a dendritic cell. Examples include GM-CSF, IL-1, TNF, IL-4, CD40L, CTLA4, CD28, and FLT-3 ligand.

For the purpose of the present invention, the term “vaccine composition” is intended to mean a composition which can be administered to humans or to animals in order to induce an immune system response; this immune system response can result in a production of antibodies or simply in the activation of certain cells, in particular antigen-presenting cells, T lymphocytes and B lymphocytes. The vaccine composition can be a composition for prophylactic purposes or for therapeutic purposes, or both. As used herein the term “antigen” refers to any antigen which can be used in a vaccine, whether it involves a whole microorganism or a subunit, and whatever its nature: peptide, protein, glycoprotein, polysaccharide, glycolipid, lipopeptide, etc. They may be viral antigens, bacterial antigens, or the like; the term “antigen” also comprises the polynucleotides, the sequences of which are chosen so as to encode the antigens whose expression by the individuals to which the polynucleotides are administered is desired, in the case of the immunization technique referred to as DNA immunization. They may also be a set of antigens, in particular in the case of a multivalent vaccine composition which comprises antigens capable of protecting against several diseases, and which is then generally referred to as a vaccine combination, or in the case of a composition which comprises several different antigens in order to protect against a single disease, as is the case for certain vaccines against whooping cough or the flu, for example. The term “antibodies” refers to immunoglobulins, whether natural or partially or wholly produced artificially, e.g. recombinant. An antibody may be monoclonal or polyclonal. The antibody may, in some cases, be a member of one, or a combination immunoglobulin classes, including: IgG, IgM, IgA, IgD, and IgE.

The term “adjuvant” refers to a substance that enhances, augments or potentiates the host's immune response to a vaccine antigen.

The term “gene” is used to refer to a functional protein, polypeptide or peptide-encoding unit. As will be understood by those in the art, this functional term includes both genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated

As used herein, the term “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

As used in this application, the term “amino acid” means one of the naturally occurring amino carboxylic acids of which proteins are comprised. The term “polypeptide” as described herein refers to a polymer of amino acid residues joined by peptide bonds, whether produced naturally or synthetically. Polypeptides of less than about 10 amino acid residues are commonly referred to as “peptides.” A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

As used herein, the term “in vivo” refers to being inside the body. The term “in vitro” used as used in the present application is to be understood as indicating an operation carried out in a non-living system.

As used herein, the term “treatment” or “treating” means any administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology).

The present invention describes novel vaccine adjuvants based on targeting adjuvants with antibodies directly to antigen-presenting cells. The present invention was found to enhance vaccine efficacy by directly linking adjuvants (e.g., TLR ligands or agonists), more specifically TLR7 ligands (TLR7L) directly to DC-targeting vaccines. As shown herein, the compositions and methods of the present invention are broadly applicable to all DC-targeting vaccines and extensible to making adjuvants with unexpected novel properties. The present invention is an adjuvant and methods for making and using the same in which the adjuvant is conjugated to a DC-targeting vaccine (e.g., anti-DC receptor antibody fused to antigen). Several aspects of the present invention have advantages over the prior art, including dose-sparing (by sending the adjuvant directly to the antigen-presenting cell that actually receives antigen), unexpectedly, this discovery provides a method of making the agonist activity of the adjuvant strictly dependent upon the type of DC receptor targeted.

Preparation of cells: Apheresis procedures were performed on healthy individuals after informed consent was collected. This protocol was reviewed and approved by the Baylor Research Institute Institutional Review Board. PBMCs were purified from apheresis blood samples and used after cryopreservation. Monocyte-derived IFNa-DCs were prepared from frozen human monocytes (elutriation fraction 5, Lemarie et al, J. Immunological Methods, 2007) cultured with GM-CSF (100 ng/ml) and IFNa (500 U/ml)) (Salluto et al, J. Exp. Med) for 3 days in Cellgenix.

Extracellular cytokine secretion assay. PBMCs or monocyte-derived IFNα-DCs (2×10⁶ cells/ml, 200 μl/well) were cultured in cRPMI containing 10% human AB serum, 2 mM L-glutamine, 50 U penicillin, 50 μg/ml streptomycin, 1× essential amino acids, 25 mM hepes, 55 μM 2-mercapto-ethanol with DC-targeting vaccines and TLR ligands of interest or left unstimulated (negative control) for 24 h, at 37° C. and 5% CO₂. Then culture supernatants were harvested and then the secreted cytokines were measured in the culture supernatants using BioPlex200 Luminex (BioRad).

The present invention describes novel vaccine adjuvants based on targeting adjuvants with antibodies directly to antigen-presenting cells. The present invention was found to enhance vaccine efficacy by directly linking adjuvants, more specifically Toll-Like Receptor (TLR) ligands directly to DC-targeting vaccines. As shown herein, the compositions and methods of the present invention are broadly applicable to all DC-targeting vaccines and extensible to making adjuvants with unexpected novel properties. While adjuvants directly linked to adjuvants are well known for e.g., development of Hep B vaccine linked to CpG (Dynavax) or Flu antigens linked to flagellin (Vaxigen), the present invention relates to adjuvants and methods for making and using the same in which the adjuvant is conjugated to a DC-targeting vaccine (e.g., anti-DC receptor antibody fused to antigen).

Several aspects of the present invention have advantages over the prior art, including dose-sparing (by sending the adjuvant directly to the antigen-presenting cell that actually receives antigen), unexpectedly, this discovery provides a method of making the agonist activity of the adjuvant strictly dependent upon the type of DC receptor targeted.

The present invention further discloses a vaccine composition comprising: (i) an antigen, (ii) an adjuvant comprising an anti-dendritic cell (DC)-specific antibody or fragment thereof conjugated to a TLR agonist, and (iii) a pharmaceutically acceptable carrier, wherein the conjugate and agonist are each comprised in an amount such that, in combination with the other, are effective to produce the immune response in a human or animal subject in need of immunostimulation.

The inventors describe a DC-targeting agent linked to a TLR7 ligand or a flagellin-based adjuvant and linked to cohesin-antigen via the dockerin domain. It must be noted that any desired antigen can also be directly fused in place of the dockerin domain. Some non-limiting examples of these constructs are presented herein below:

(SEQ ID NO: 1)

C566 E. coli-pET28 vector encoding expression of
[Cohesin-var1-FluM1-6xHis]
MDLDAVRIKVDTVNAKPGDTVNIPVRFSGIPSKGIANADFVYSYDPNVLE
IIEIKPGELIVDPNPTKSFDTAVYPDRKMIVFLFAEDSGTGAYAITKDGV
FATIVAKVKEGAPNGLSVIKFVEVGGFANNDLVEQKTQFFDGGVNVGDTT
EPATPTTPVTTPTTTDDLDA [symbol] [symbol] LLTEVETYVLSIIPSGPLKAEIAQ
RLEDVFAGKNTDLEVLMEWLKTRPILSPLTKGILGFVFTLTVPSERGLQR
RRFVQNALNGNGDPNNMDKAVKLYRKLKREITFHGAKEIALSYSAGAL [symbol]
CMGLIYNRMGAVTTEVAFGLVCATCEQIADSQHRSHRQMVTTTNPLIRHE
NRMVL [symbol] TTAKAMEQMAGSSEQAAEAMDIASQARQMVQAMRTIGTHPSSS
AGLKDDLLENLQAYQKRMGVQMQRFKLEHHHHHH

Bold is the cohesin domain showing an underlined single C to A change that maintains dockerin binding and 3 C residues (bold-underlined) that permit site-specific maleimide linkage of TLRL adducts. Underlined is the Flu M1 antigen sequence. AS sequences in bold-italics are joining sequences from construction of the expression vector.

In related forms any cohesin-antigen with free cys residues can be conveniently decorated with TLR7-L compound and linked with any anti-DC receptor-dockerin-antigen vaccine.

C1450 encodes mouse Anti-DCIR_9E8_H-LV-hIgG4H-C-
Flex-v1-v1C2 (SEQ ID NO: 2):
QVTLKESGPGILQPSQTLSLTCSFSGFSLSTSGMGLSWIRQPSGKGLEWL
AHIYWDDDKRYNPSLKSRLTISKDTSSNQVFLKITIVDTADAATYYCARS
SHYYGYGYGGYFDVWGAGTTVTVSSAKTKGPSVFPLAPCSRSTSESTAAL
GCLVKDYFPEPVTVSWNSGALTSGVHTFPAVLQSSGLYSLSSVVTVPSSS
LGTKTYTCNVDHKPSNTKVDKRVESKYGPPCPPCPAPEFEGGPSVFLFPP
KPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLSLSL
GK [symbol] QTPTNTISVTPTNNSTPTNNSNPKPNP [symbol] CQTPTNTISVTPTNNST
PTNNSNPKPCP

[symbol]

Bold is a flexible linker sequence bearing two C residues (underlined) for site-specific linking TLRL adducts. When co-transfected with appropriate L chain expression vector into mammalian cells (e.g., CHO-S cells) this vector directs efficient secretion of a typical embodiment of a DC-targeting agent linked to a chemical-based adjuvant. In related embodiments other vectors can be prepared with any desired antigen directly fused to the C-terminal codon. AS sequences in bold-italics are joining sequences from construction of the expression vector. Italics is a flexible linker (supra).

Some other non-limiting examples of constructs with different DC-specific antibodies or fragments (both nucleotide and protein sequences) are presented herein below:

Anti_CLEC_6_9B9.2G12_Hv-V-hIgG4H-C (SEQ ID NO: 3):
ATGGGCAGGCTTACTTCTTCATTCTTGCTACTGATTGTCCCTGCATATGTCCTGTCCC
AGGTTACTCTGAAAGAGTCTGGCCCTGGGATATTGCAGCCCTCCCAGACCCTCAGTC
TGACCTGTTCTTTCTCTGGGTTTTCACTGAGCACTTCTGGTATGAGTGTAGGCTGGAT
TCGTCAGCCTTCAGGGAAGGGTCTGGAGTGGCTGGCTCACATTTGGTGGAATGATGA
TAAGTACTATAATCCAGTCCTGAAAAGCCGGCTCACAATCTCCAAGGAGACCTCCA
ACAACCAGGTATTCCTCAAGATCGCCAGTGTGGTCTCTGCAGATACTGCCACATACT
ACTGTGCTCGATTCTATGGTAACTGTCTTGACTACTGGGGCCAAGGCACCACTCTCA
CAGTCTCCTCGGCCAAAACAaagggcccATCCGTCTTCCCCCTGGCGCCCTGCTCCAGGA
GCACCTCCGAGAGCACAGCCGCCCTGGGCTGCCTGGTCAAGGACTACTTCCCCGAA
CCGGTGACGGTGTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCCCTC
CAGCAGCTTGGGCACGAAGACCTACACCTGCAACGTAGATCACAAGCCCAGCAACA
CCAAGGTGGACAAGAGAGTTGAGTCCAAATATGGTCCCCCATGCCCACCCTGCCCA
GCACCTGAGTTCGAAGGGGGACCATCAGTCTTCCTGTTCCCCCCAAAACCCAAGGAC
ACTCTCATGATCTCCCGGACCCCTGAGGTCACGTGCGTGGTGGTGGACGTGAGCCAG
GAAGACCCCGAGGTCCAGTTCAACTGGTACGTGGATGGCGTGGAGGTGCATAATGC
CAAGACAAAGCCGCGGGAGGAGCAGTTCAACAGCACGTACCGTGTGGTCAGCGTCC
TCACCGTCCTGCACCAGGACTGGCTGAACGGCAAGGAGTACAAGTGCAAGGTCTCC
AACAAAGGCCTCCCGTCCTCCATCGAGAAAACCATCTCCAAAGCCAAAGGGCAGCC
CCGAGAGCCACAGGTGTACACCCTGCCCCCATCCCAGGAGGAGATGACCAAGAACC
AGGTCAGCCTGACCTGCCTGGTCAAAGGCTTCTACCCCAGCGACATCGCCGTGGAGT
GGGAGAGCAATGGGCAGCCGGAGAACAACTACAAGACCACGCCTCCCGTGCTGGAC
TCCGACGGCTCCTTCTTCCTCTACAGCAGGCTAACCGTGGACAAGAGCAGGTGGCAG
GAGGGGAATGTCTTCTCATGCTCCGTGATGCATGAGGCTCTGCACAACCACTACACA
CAGAAGAGCCTCTCCCTGTCTCTGGGTAAAGCTAGCTGATTAATTAA
Anti_CLEC_6_9B9.2G12_Hv-V-hIgG4H-C (SEQ ID NO: 68):
MGRLTSSFLLLIVPAYVLSQVTLKESGPGILQPSQTLSLTCSFSGFSLSTSGMSVGWIRQPS
GKGLEWLAHIWWNDDKYYNPVLKSRLTISKETSNNQVFLKIASVVSADTATYYCARFY
GNCLDYWGQGTTLTVSSAKTKGPSVFPLAPCSRSTSESTAALGCLVKDYFPEPVTVSWN
SGALTSGVHTFPAVLQSSGLYSLSSVVTVPSSSLGTKTYTCNVDHKPSNTKVDKRVESK
YGPPCPPCPAPEFEGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGLPSSIEKTISKAK
GQPREPQVYTLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGKAS
Anti_CLEC_6_9B9.2G12_Kv-V-hIgGK-C (SEQ ID NO: 4):
ATGATGTCCTCTGCTCAGTTCCTTGGTCTCCTGTTGCTCTGTTTTCAAGGTACCAGAT
GTGATATCCAGATGACACAGACTACATCCTCCCTGTCTGCCTCTCTGGGAGACAGAG
TCACCATCAGTTGCAGGGCAAGTCAGGACATTAGCAATTATTTAAACTGGTATCAGC
AGAAACCAGATGGAACTGTTAAACTCCTGATCTACTACACATCAATATTACAATTAG
GAGTCCCATCAAGATTCAGTGGCAGTGGGTCTGAAACAGATTATTCTCTCACCATTA
GCAACCTGGAGCAAGAAGATATTGCCACTTACTTTTGCCAACAGGGTGATTCGCTTC
CATTCACGTTCGGCTCGGGGACAAAGCTCGAGATCAAACGAACTGTGGCTGCACCA
TCTGTCTTCATCTTCCCGCCATCTGATGAGCAGTTGAAATCTGGAACTGCCTCTGTTG
TGTGCCTGCTGAATAACTTCTATCCCAGAGAGGCCAAAGTACAGTGGAAGGTGGAT
AACGCCCTCCAATCGGGTAACTCCCAGGAGAGTGTCACAGAGCAGGACAGCAAGGA
CAGCACCTACAGCCTCAGCAGCACCCTGACGCTGAGCAAAGCAGACTACGAGAAAC
ACAAAGTCTATGCCTGCGAAGTCACCCATCAGGGCCTGAGCTCGCCCGTCACAAAG
AGCTTCAACAGGGGAGAGTGTTAG
Anti_CLEC_6_9B9.2G12_Kv-V-hIgGK-C (SEQ ID NO: 69):
MMSSAQFLGLLLLCFQGTRCDIQMTQTTSSLSASLGDRVTISCRASQDISNYLNWYQQK
PDGTVKLLIYYTSILQLGVPSRFSGSGSETDYSLTISNLEQEDIATYFCQQGDSLPFTFGSG
TKLEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
Anti-ASGPR_49C11_7H-LV-hIgG4H-C (SEQ ID NO: 5):
ATGAGAGCGCTGATTCTTTTGTGCCTGTTCACAGCCTTTCCTGGTATCCTGTCTGATG
TGCAGCTTCAGGAGTCAGGACCTGACCTGGTGAAACCTTCTCAGTCACTTTCACTCA
CCTGCACTGTCACTGGCTACTCCATCACCAGTGGTTATAGCTGGCACTGGATCCGGC
AGTTTCCAGGAAACAAACTGGAATGGATGGGCTACATACTCTTCAGTGGTAGCACTA
ACTACAACCCATCTCTGAAAAGTCGAATCTCTATCACTCGAGACACATCCAAGAACC
AGTTCTTCCTGCAGTTGAATTCTGTGACTACTGAGGACACAGCCACATATTTCTGTGC
AAGATCTAACTATGGTTCCTTTGCTTCCTGGGGCCAAGGGACTCTGGTCACTGTCTCT
GCAGCCAAAACAACGGGCCCATCCGTCTTCCCCCTGGCGCCCTGCTCCAGGAGCACC
TCCGAGAGCACAGCCGCCCTGGGCTGCCTGGTCAAGGACTACTTCCCCGAACCGGT
GACGGTGTCGTGGAACTCAGGCGCCCTGACCAGCGGCGTGCACACCTTCCCGGCTGT
CCTACAGTCCTCAGGACTCTACTCCCTCAGCAGCGTGGTGACCGTGCCCTCCAGCAG
CTTGGGCACGAAGACCTACACCTGCAACGTAGATCACAAGCCCAGCAACACCAAGG
TGGACAAGAGAGTTGAGTCCAAATATGGTCCCCCATGCCCACCCTGCCCAGCACCTG
AGTTCGAAGGGGGACCATCAGTCTTCCTGTTCCCCCCAAAACCCAAGGACACTCTCA
TGATCTCCCGGACCCCTGAGGTCACGTGCGTGGTGGTGGACGTGAGCCAGGAAGAC
CCCGAGGTCCAGTTCAACTGGTACGTGGATGGCGTGGAGGTGCATAATGCCAAGAC
AAAGCCGCGGGAGGAGCAGTTCAACAGCACGTACCGTGTGGTCAGCGTCCTCACCG
TCCTGCACCAGGACTGGCTGAACGGCAAGGAGTACAAGTGCAAGGTCTCCAACAAA
GGCCTCCCGTCCTCCATCGAGAAAACCATCTCCAAAGCCAAAGGGCAGCCCCGAGA
GCCACAGGTGTACACCCTGCCCCCATCCCAGGAGGAGATGACCAAGAACCAGGTCA
GCCTGACCTGCCTGGTCAAAGGCTTCTACCCCAGCGACATCGCCGTGGAGTGGGAG
AGCAATGGGCAGCCGGAGAACAACTACAAGACCACGCCTCCCGTGCTGGACTCCGA
CGGCTCCTTCTTCCTCTACAGCAGGCTAACCGTGGACAAGAGCAGGTGGCAGGAGG
GGAATGTCTTCTCATGCTCCGTGATGCATGAGGCTCTGCACAACCACTACACACAGA
AGAGCCTCTCCCTGTCTCTGGGTAAAGCTAGCTGATTAATTAA
Anti-ASGPR_49C11_7H-LV-hIgG4H-C (SEQ ID NO: 70):
MRALILLCLFTAFPGILSDVQLQESGPDLVKPSQSLSLTCTVTGYSITSGYSWHWIRQFPG
NKLEWMGYILFSGSTNYNPSLKSRISITRDTSKNQFFLQLNSVTTEDTATYFCARSNYGS
FASWGQGTLVTVSAAKTTGPSVFPLAPCSRSTSESTAALGCLVKDYFPEPVTVSWNSGA
LTSGVHTFPAVLQSSGLYSLSSVVTVPSSSLGTKTYTCNVDHKPSNTKVDKRVESKYGPP
CPPCPAPEFEGGPSVFLFPPKPKDTLMISRTPEVTCVVVDVSQEDPEVQFNWYVDGVEV
HNAKTKPREEQFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGKAS
Anti-ASGPR_49C11_7K-LV-hIgGK-C (SEQ ID NO: 6):
ATGGATTTTCAAGTGCAGATTTTCAGCTTCCTGCTAATCAGTGCCTCAGTCATAATAT
CCAGAGGACAAATTGTTCTCACCCAGTCTCCAGCAATCATGTCTGCATCTCCAGGGG
AGAAGGTCACCATGACCTGCAGTGCCAGCTCAAGTGTAAGTCACATGCACTGGTAC
CAGCAGAAGTCAGGCACTTCCCCCAAAAGATGGATTTATGACACATCCAGACTGGC
TTCTGGAGTCCCTGCTCGCTTCAGTGGCAGTGGGTCTGGGACCTCTTACTCTCTCACA
ATCAGCAGCATGGAGGCTGAAGATGCTGCCACTTATTACTGCCAGCAGTGGAGTAG
TCACCCATGGTCGTTCGGTGGAGGCACCAAACTCGAGATCAAACGAACTGTGGCTG
CACCATCTGTCTTCATCTTCCCGCCATCTGATGAGCAGTTGAAATCTGGAACTGCCTC
TGTTGTGTGCCTGCTGAATAACTTCTATCCCAGAGAGGCCAAAGTACAGTGGAAGGT
GGATAACGCCCTCCAATCGGGTAACTCCCAGGAGAGTGTCACAGAGCAGGACAGCA
AGGACAGCACCTACAGCCTCAGCAGCACCCTGACGCTGAGCAAAGCAGACTACGAG
AAACACAAAGTCTATGCCTGCGAAGTCACCCATCAGGGCCTGAGCTCGCCCGTCAC
AAAGAGCTTCAACAGGGGAGAGTGTTAG
Anti-ASGPR_49C11_7K-LV-hIgGK-C (SEQ ID NO: 71):
MDFQVQIFSFLLISASVIISRGQIVLTQSPAIMSASPGEKVTMTCSASSSVSHMHWYQQKS
GTSPKRWIYDTSRLASGVPARFSGSGSGTSYSLTISSMEAEDAATYYCQQWSSHPWSFG
GGTKLEIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
Anti-ASGPR_4G2.2_Hv-V-hIgG4H-C (SEQ ID NO: 7):
ATGGCTTGGGTGTGGACCTTGCTATTCCTGATGGCAGCTGCCCAAAGTGCCCAAGCA
CAGATCCAGTTGGTGCAGTCTGGACCTGAGCTGAAGAAGCCTGGAGAGACAGTCAA
GATCTCCTGCAAGGCTTCTGGGTATACCTTCACAAACTATGGAATGAACTGGGTGAA
GCAGGTTCCAGGAAAAGGTTTAAGGTGGATGGGCTGGATGGACACCTTCACTGGAG
AGCCAACATATGCTGATGACTTCAAGGGACGGTTTGCCTTCTCTTTGGAAACCTCTG
CCAGCACTGCCTATTTGCAGATCAACAGCCTCAAAAATGAGGACACGGCTACTTATT
TCTGTGCAAGAGGGGGGATTTTACGACTCAACTACTTTGACTACTGGGGCCAAGGCA
CCACTCTCACAGTCTCCTCAGCCAAAACGAAGGGCCCATCCGTCTTCCCCCTGGCGC
CCTGCTCCAGGAGCACCTCCGAGAGCACAGCCGCCCTGGGCTGCCTGGTCAAGGAC
TACTTCCCCGAACCGGTGACGGTGTCGTGGAACTCAGGCGCCCTGACCAGCGGCGT
GCACACCTTCCCGGCTGTCCTACAGTCCTCAGGACTCTACTCCCTCAGCAGCGTGGT
GACCGTGCCCTCCAGCAGCTTGGGCACGAAGACCTACACCTGCAACGTAGATCACA
AGCCCAGCAACACCAAGGTGGACAAGAGAGTTGAGTCCAAATATGGTCCCCCATGC
CCACCCTGCCCAGCACCTGAGTTCGAAGGGGGACCATCAGTCTTCCTGTTCCCCCCA
AAACCCAAGGACACTCTCATGATCTCCCGGACCCCTGAGGTCACGTGCGTGGTGGTG
GACGTGAGCCAGGAAGACCCCGAGGTCCAGTTCAACTGGTACGTGGATGGCGTGGA
GGTGCATAATGCCAAGACAAAGCCGCGGGAGGAGCAGTTCAACAGCACGTACCGTG
TGGTCAGCGTCCTCACCGTCCTGCACCAGGACTGGCTGAACGGCAAGGAGTACAAG
TGCAAGGTCTCCAACAAAGGCCTCCCGTCCTCCATCGAGAAAACCATCTCCAAAGCC
AAAGGGCAGCCCCGAGAGCCACAGGTGTACACCCTGCCCCCATCCCAGGAGGAGAT
GACCAAGAACCAGGTCAGCCTGACCTGCCTGGTCAAAGGCTTCTACCCCAGCGACA
TCGCCGTGGAGTGGGAGAGCAATGGGCAGCCGGAGAACAACTACAAGACCACGCCT
CCCGTGCTGGACTCCGACGGCTCCTTCTTCCTCTACAGCAGGCTAACCGTGGACAAG
AGCAGGTGGCAGGAGGGGAATGTCTTCTCATGCTCCGTGATGCATGAGGCTCTGCAC
AACCACTACACACAGAAGAGCCTCTCCCTGTCTCTGGGTAAAGCTAGCTGATTAATT
AA
Anti-ASGPR_4G2.2_Hv-V-hIgG4H-C (SEQ ID NO: 72):
MAWVWTLLFLMAAAQSAQAQIQLVQSGPELKKPGETVKISCKASGYTFTNYGMNWVK
QVPGKGLRWMGWMDTFTGEPTYADDFKGRFAFSLETSASTAYLQINSLKNEDTATYFC
ARGGILRLNYFDYWGQGTTLTVSSAKTKGPSVFPLAPCSRSTSESTAALGCLVKDYFPEP
VTVSWNSGALTSGVHTFPAVLQSSGLYSLSSVVTVPSSSLGTKTYTCNVDHKPSNTKVD
KRVESKYGPPCPPCPAPEFEGGPSVFLFPPKPKDTLMISRTPEVTCVVVDVSQEDPEVQF
NWYVDGVEVHNAKTKPREEQFNSTYRVVSVLTVLHQDWLNGKEYKCKVSNKGLPSSI
EKTISKAKGQPREPQVYTLPPSQEEMTKNQVSLTCLVKGFYPSDIAVEWESNGQPENNY
KTTPPVLDSDGSFFLYSRLTVDKSRWQEGNVFSCSVMHEALHNHYTQKSLSLSLGKAS
Anti-ASGPR_4G2.2_Kv-V-hIgGK-C (SEQ ID NO: 8):
ATGAAGTTTCCTTCTCAACTTCTGCTCTTACTGCTGTTTGGAATCCCAGGCATGATAT
GTGACATCCAGATGACACAATCTTCATCCTCCTTTTCTGTATCTCTAGGAGACAGAG
TCACCATTACTTGCAAGGCAAGTGAGGACATATATAATCGGTTAGGCTGGTATCAGC
AGAAACCAGGAAATGCTCCTAGGCTCTTAATATCTGGTGCAACCAGTTTGGAAACTG
GGGTTCCTTCAAGATTCAGTGGCAGTGGATCTGGAAAGGATTACGCTCTCAGCATTA
CCAGTCTTCAGACTGAAGATCTTGCTACTTATTACTGTCAACAGTGTTGGACTTCTCC
GTACACGTTCGGAGGGGGGACCAAGCTCGAGATCAAACGAACTGTGGCTGCACCAT
CTGTCTTCATCTTCCCGCCATCTGATGAGCAGTTGAAATCTGGAACTGCCTCTGTTGT
GTGCCTGCTGAATAACTTCTATCCCAGAGAGGCCAAAGTACAGTGGAAGGTGGATA
ACGCCCTCCAATCGGGTAACTCCCAGGAGAGTGTCACAGAGCAGGACAGCAAGGAC
AGCACCTACAGCCTCAGCAGCACCCTGACGCTGAGCAAAGCAGACTACGAGAAACA
CAAAGTCTATGCCTGCGAAGTCACCCATCAGGGCCTGAGCTCGCCCGTCACAAAGA
GCTTCAACAGGGGAGAGTGTTAG
Anti-ASGPR_4G2.2_Kv-V-hIgGK-C (SEQ ID NO: 73):
MKFPSQLLLLLLFGIPGMICDIQMTQSSSSFSVSLGDRVTITCKASEDIYNRLGWYQQKPG
NAPRLLISGATSLETGVPSRFSGSGSGKDYALSITSLQTEDLATYYCQQCWTSPYTFGGG
TKLEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
Anti-ASGPR_5F10H-LV-hIgG4H-C (SEQ ID NO: 9):
ATGGGATGGAGCTGGATCTTTCTCTTTCTCTTGTCAGGAACTGGAGGTGTCCTCTCTG
AGGTCCAGCTGCAACAGTCTGGACCTGAGCTGGTGAAGCCTGGGGCTTCAGTGAAG
ATGTCCTGCAAGGCTTCTGGATACACCTTCACTGACTACTACATGAAGTGGGTGAAG
CAGAGCCATGGAAAGAGCCTTGAGTGGATTGGAGATATTAATCCTAACTATGGTGA
TACTTTCTACAACCAGAAGTTCGAGGGCAAGGCCACATTGACTGTAGACAAATCCTC
CAGGACAGCCTACATGCAGCTCAACAGCCTGACATCTGAGGACTCTGCAGTCTATTA
TTGTGGAAGAGGGGACTATGGATACTTCGATGTCTGGGGCGCAGGGACCACGGTCA
CCGTCTCCTCAGCCAAAACAAAGGGCCCATCCGTCTTCCCCCTGGCGCCCTGCTCCA
GGAGCACCTCCGAGAGCACAGCCGCCCTGGGCTGCCTGGTCAAGGACTACTTCCCC
GAACCGGTGACGGTGTCGTGGAACTCAGGCGCCCTGACCAGCGGCGTGCACACCTT
CCCGGCTGTCCTACAGTCCTCAGGACTCTACTCCCTCAGCAGCGTGGTGACCGTGCC
CTCCAGCAGCTTGGGCACGAAGACCTACACCTGCAACGTAGATCACAAGCCCAGCA
ACACCAAGGTGGACAAGAGAGTTGAGTCCAAATATGGTCCCCCATGCCCACCCTGC
CCAGCACCTGAGTTCGAAGGGGGACCATCAGTCTTCCTGTTCCCCCCAAAACCCAAG
GACACTCTCATGATCTCCCGGACCCCTGAGGTCACGTGCGTGGTGGTGGACGTGAGC
CAGGAAGACCCCGAGGTCCAGTTCAACTGGTACGTGGATGGCGTGGAGGTGCATAA
TGCCAAGACAAAGCCGCGGGAGGAGCAGTTCAACAGCACGTACCGTGTGGTCAGCG
TCCTCACCGTCCTGCACCAGGACTGGCTGAACGGCAAGGAGTACAAGTGCAAGGTC
TCCAACAAAGGCCTCCCGTCCTCCATCGAGAAAACCATCTCCAAAGCCAAAGGGCA
GCCCCGAGAGCCACAGGTGTACACCCTGCCCCCATCCCAGGAGGAGATGACCAAGA
ACCAGGTCAGCCTGACCTGCCTGGTCAAAGGCTTCTACCCCAGCGACATCGCCGTGG
AGTGGGAGAGCAATGGGCAGCCGGAGAACAACTACAAGACCACGCCTCCCGTGCTG
GACTCCGACGGCTCCTTCTTCCTCTACAGCAGGCTAACCGTGGACAAGAGCAGGTGG
CAGGAGGGGAATGTCTTCTCATGCTCCGTGATGCATGAGGCTCTGCACAACCACTAC
ACACAGAAGAGCCTCTCCCTGTCTCTGGGTAAAGCTAGCTGA
Anti-ASGPR_5F10H-LV-hIgG4H-C (SEQ ID NO: 74):
MGWSWIFLFLLSGTGGVLSEVQLQQSGPELVKPGASVKMSCKASGYTFTDYYMKWVK
QSHGKSLEWIGDINPNYGDTFYNQKFEGKATLTVDKSSRTAYMQLNSLTSEDSAVYYC
GRGDYGYFDVWGAGTTVTVSSAKTKGPSVFPLAPCSRSTSESTAALGCLVKDYFPEPVT
VSWNSGALTSGVHTFPAVLQSSGLYSLSSVVTVPSSSLGTKTYTCNVDHKPSNTKVDKR
VESKYGPPCPPCPAPEFEGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIEKT
ISKAKGQPREPQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLLSLGKAS
Anti-ASGPR_5F10K-LV-hIgGK-C (SEQ ID NO: 10):
ATGGAGACACATTCTCAGGTCTTTGTATACATGTTGCTGTGGTTGTCTGGTGTTGAAG
GAGACATTGTGATGACCCAGTCTCACAAATTCATGTCCACATCAGTAGGAGACAGG
GTCAGCATCACCTGCAAGGCCAGTCAGGATGTGGGTACTGCTGTAGCCTGGTATCAA
CAGAAACCAGGGCAATCTCCTAAACTACTGATTTACTGGGCATCCACCCGGCACACT
GGAGTCCCTGATCGCTTCACAGGCAGTGGATCTGGGACAGATTTCACTCTCACCATT
AACAATGTGCAGTCTGAAGACTTGGCAGATTATTTCTGTCAGCAATATAGCAGCAAT
CCGTACATGTTCGGAGGGGGGACCAAGCTCGAGATCAAACGAACTGTGGCTGCACC
ATCTGTCTTCATCTTCCCGCCATCTGATGAGCAGTTGAAATCTGGAACTGCCTCTGTT
GTGTGCCTGCTGAATAACTTCTATCCCAGAGAGGCCAAAGTACAGTGGAAGGTGGA
TAACGCCCTCCAATCGGGTAACTCCCAGGAGAGTGTCACAGAGCAGGACAGCAAGG
ACAGCACCTACAGCCTCAGCAGCACCCTGACGCTGAGCAAAGCAGACTACGAGAAA
CACAAAGTCTATGCCTGCGAAGTCACCCATCAGGGCCTGAGCTCGCCCGTCACAAA
GAGCTTCAACAGGGGAGAGTGTTAG
Anti-ASGPR_5F10K-LV-hIgGK-C (SEQ ID NO: 75):
METHSQVFVYMLLWLSGVEGDIVMTQSHKFMSTSVGDRVSITCKASQDVGTAVAWYQ
QKPGQSPKLLIYWASTRHTGVPDRFTGSGSGTDFTLTINNVQSEDLADYFCQQYSSNPY
MFGGGTKLEIKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTKSFNRGEC
Anti-ASGPR1H11_H-V-hIgG4H-C (SEQ ID NO: 11):
ATGGGATGGAGCTGGATCTTTCTCTTTCTCCTGTCAGGAACTGCAGGTGTCCTCTCTG
AGGTCCAGCTGCAACAGTCTGGACCTGAGTTGGTGAAGCCTGGGGCTTCAGTGAAG
ATATCCTGCAAGACTTCTGGATACACATTCACTGAATACACCATGCACTGGGTGAGG
CAGAGCCATGGAAAGAGCCTTGAGTGGATTGGAGGTATTAATCCTATCAATGGTGG
TCCTACCTACAACCAGAAGTTCAAGGGCAAGGCCACATTGACTGTTGACAAGTCCTC
CAGCACAGCCTACATGGAGCTCCGCAGCCTGACATCTGAGGACTCTGCAGTCTATTA
CTGTGCAAGATGGGACTATGGTAGTCGAGATGTTATGGACTACTGGGGTCAAGGAA
CCTCAGTCACCGTCTCCTCAGCCAAAACGAAGGGCCCATCCGTCTTCCCCCTGGCGC
CCTGCTCCAGGAGCACCTCCGAGAGCACAGCCGCCCTGGGCTGCCTGGTCAAGGAC
TACTTCCCCGAACCGGTACCGGTGACGGTGTCGTGGAACTCAGGCGCCCTGACCAGC
GGCGTGCACACCTTCCCGGCTGTCCTACAGTCCTCAGGACTCTACTCCCTCAGCAGC
GTGGTGACCGTGCCCTCCAGCAGCTTGGGCACGAAGACCTACACCTGCAACGTAGA
TCACAAGCCCAGCAACACCAAGGTGGACAAGAGAGTTGAGTCCAAATATGGTCCCC
CATGCCCACCCTGCCCAGCACCTGAGTTCGAAGGGGGACCATCAGTCTTCCTGTTCC
CCCCAAAACCCAAGGACACTCTCATGATCTCCCGGACCCCTGAGGTCACGTGCGTGG
TGGTGGACGTGAGCCAGGAAGACCCCGAGGTCCAGTTCAACTGGTACGTGGATGGC
GTGGAGGTGCATAATGCCAAGACAAAGCCGCGGGAGGAGCAGTTCAACAGCACGTA
CCGTGTGGTCAGCGTCCTCACCGTCCTGCACCAGGACTGGCTGAACGGCAAGGAGT
ACAAGTGCAAGGTCTCCAACAAAGGCCTCCCGTCCTCCATCGAGAAAACCATCTCC
AAAGCCAAAGGGCAGCCCCGAGAGCCACAGGTGTACACCCTGCCCCCATCCCAGGA
GGAGATGACCAAGAACCAGGTCAGCCTGACCTGCCTGGTCAAAGGCTTCTACCCCA
GCGACATCGCCGTGGAGTGGGAGAGCAATGGGCAGCCGGAGAACAACTACAAGAC
CACGCCTCCCGTGCTGGACTCCGACGGCTCCTTCTTCCTCTACAGCAGGCTAACCGT
GGACAAGAGCAGGTGGCAGGAGGGGAATGTCTTCTCATGCTCCGTGATGCATGAGG
CTCTGCACAACCACTACACACAGAAGAGCCTCTCCCTGTCTCTGGGTAAAGCTAGCT
GA
Anti-ASGPR1H11_H-V-hIgG4H-C (SEQ ID NO: 76):
MGWSWIFLFLLSGTAGVLSEVQLQQSGPELVKPGASVKISCKTSGYTFTEYTMHWVRS
HGKSLEWIGGINPINGGPTYNQKFKGKATLTVDKSSSTAYMELRSLTSEDSAVYYCARW
DYGSRDVMDYWGQGTSVTVSSAKTKGPSVFPLAPCSRSTSESTAALGCLVKDYFPEPVP
VTVSWNSGALTSGVHTFPAVLQSSGLYSLSSVVTVPSSSLGTKTYTCNVDHKPSNTKVD
KRVESKYGPPCPPCPAPEFEGGPSVFLFPPKPKDTLMISRTPEVTCVVVDVSQEDPEVQF
NWYVDGVEVHNAKTKPREEQFNSTYRVVSVLTVLHQDWLNGKEYKCKVSNKGLPSSI
EKTISKAKGQPREPQVYTLPPSQEEMTKNQVSLTCLVKGFYPSDIAVEWESNGQPENNY
KTTPPVLDSDGSFFLYSRLTVDKSRWQEGNVFSCSVMHEALHNHYTQKSLSLSLGKAS
Anti-ASGPR1H11K-LV-var2-hIgGK-C (SEQ ID NO: 12):
ATGGAATCACAGACTCTGGTCTTCATATCCATACTGCTCTGGTTATATGGTGCTGATG
GGAACATTGTAATGACTCAATCTCCCAAATCCATGTCCATGTCAGTAGGGGAGAGG
GTCACCTTGAGCTGCAAGGCCAGTGAGAATGTGGGAACTTATGTATCCTGGTATCAA
CAGAGACCAGAACAGTCTCCAAAACTGCTGATATACGGGGCATCCAACCGGTACAC
TGGGGTCCCCGATCGCTTCACAGGCAGTGGATCTGCAACAGATTTCACTCTGACCAT
CAGCAGTGTGCAGGCTGAGGACCTTGCAGATTATCACTGTGGACAGACTTACAGCTA
TATATTCACGTTCGGCTCGGGGACAAAGCTCGAGATCAAACGAACTGTGGCTGCACC
ATCTGTCTTCATCTTCCCGCCATCTGATGAGCAGTTGAAATCTGGAACTGCCTCTGTT
GTGTGCCTGCTGAATAACTTCTATCCCAGAGAGGCCAAAGTACAGTGGAAGGTGGA
TAACGCCCTCCAATCGGGTAACTCCCAGGAGAGTGTCACAGAGCAGGACAGCAAGG
ACAGCACCTACAGCCTCAGCAGCACCCTGACGCTGAGCAAAGCAGACTACGAGAAA
CACAAAGTCTATGCCTGCGAAGTCACCCATCAGGGCCTGAGCTCGCCCGTCACAAA
GAGCTTCAACAGGGGAGAGTGTTAG
Anti-ASGPR1H11K-LV-var2-hIgGK-C (SEQ ID NO: 77):
METHSQVFVYMLLWLSGVEGNIVMTQSPKSMSMSVGERVTLSCKASENVGTYVSWYQ
QRPEQSPKLLIYGASNRYTGVPDRFTGSGSATDFTLTISSVQAEDLADYHCGQTYSYIFTF
GSGTKLEIKRTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSFNRGEC
Anti-CD1d_2B5.3G10_H-V-hIgG4H-C (SEQ ID NO: 13):
ATGGGATGGAGCCGGATCTTTCTCTTCCTCCTGTCAATAACTGCAGGTGTCCATTGCC
AGGTCCAGGTGCAGCAGTCGGGACCTGAGTTGGTGAAGCCTGGGGCCTCAGTGAAG
ATTTCCTGCAAAGCCTCTGGCGACGCATTCAGTAGTTCTTGGATGAACTGGGTGAAG
CAGAGGCCTGGACAGGGTCTTGAGTGGATTGGACGGATTTATCTTGGAGATGGAGA
TATTAATTACAATGGGAAGTTCAAGGGCAGGGCCACACTGACTGCAGACAAATCCT
CCAGCACAGCCTACATGCAGCTCAGCAGCCTGACCTCTGTGGACTCTGCGGTCTATT
TCTGCGCGAGGCAGCTCGGGCTATGGTATGTTATGGACTACTGGGGTCAAGGAACCT
CAGTCACCGTCTCCTCAGCCAAAACAAAGGGCCCATCCGTCTTCCCCCTGGCGCCCT
GCTCCAGGAGCACCTCCGAGAGCACAGCCGCCCTGGGCTGCCTGGTCAAGGACTAC
TTCCCCGAACCGGTGACGGTGTCGTGGAACTCAGGCGCCCTGACCAGCGGCGTGCA
CACCTTCCCGGCTGTCCTACAGTCCTCAGGACTCTACTCCCTCAGCAGCGTGGTGAC
CGTGCCCTCCAGCAGCTTGGGCACGAAGACCTACACCTGCAACGTAGATCACAAGC
CCAGCAACACCAAGGTGGACAAGAGAGTTGAGTCCAAATATGGTCCCCCATGCCCA
CCCTGCCCAGCACCTGAGTTCGAAGGGGGACCATCAGTCTTCCTGTTCCCCCCAAAA
CCCAAGGACACTCTCATGATCTCCCGGACCCCTGAGGTCACGTGCGTGGTGGTGGAC
GTGAGCCAGGAAGACCCCGAGGTCCAGTTCAACTGGTACGTGGATGGCGTGGAGGT
GCATAATGCCAAGACAAAGCCGCGGGAGGAGCAGTTCAACAGCACGTACCGTGTGG
TCAGCGTCCTCACCGTCCTGCACCAGGACTGGCTGAACGGCAAGGAGTACAAGTGC
AAGGTCTCCAACAAAGGCCTCCCGTCCTCCATCGAGAAAACCATCTCCAAAGCCAA
AGGGCAGCCCCGAGAGCCACAGGTGTACACCCTGCCCCCATCCCAGGAGGAGATGA
CCAAGAACCAGGTCAGCCTGACCTGCCTGGTCAAAGGCTTCTACCCCAGCGACATC
GCCGTGGAGTGGGAGAGCAATGGGCAGCCGGAGAACAACTACAAGACCACGCCTC
CCGTGCTGGACTCCGACGGCTCCTTCTTCCTCTACAGCAGGCTAACCGTGGACAAGA
GCAGGTGGCAGGAGGGGAATGTCTTCTCATGCTCCGTGATGCATGAGGCTCTGCACA
ACCACTACACACAGAAGAGCCTCTCCCTGTCTCTGGGTAAAGCTAGCTGA
Anti-CD1d_2B5.3G10_H-V-hIgG4H-C (SEQ ID NO: 78):
MGWSRIFLFLLSITAGVHCQVQVQQSGPELVKPGASVKISCKASGDAFSSSWMNWVKQ
RPGQGLEWIGRIYLGDGDINYNGKFKGRATLTADKSSSTAYMQLSSLTSVDSAVYFCAR
QLGLWYVMDYWGQGTSVTVSSAKTKGPSVFPLAPCSRSTSESTAALGCLVKDYFPEPV
TVSWNSGALTSGVHTFPAVLQSSGLYSLSSVVTVPSSSLGTKTYTCNVDHKPSNTKVDK
RVESKYGPPCPPCPAPEFEGGPSVFLFPPKPKDTLMISRTPEVTCVVVDVSQEDPEVQFN
WYVDGVEVHNAKTKPREEQFNSTYRVVSVLTVLHQDWLNGKEYKCKVSNKGLPSSIE
KTISKAKGQPREPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSLSLSLGKAS
Anti-CD1d_2B5.3G10_K-V-hIgGK-C (SEQ ID NO: 14):
ATGAGTGTGCCCACTCAGGTCCTGGGGTTGCTGCTGCTGTGGCTTACAGGTGCCAGA
TGTGACATCCAGATGGCTCAGTCTCCAGCCTCCCTATCTGCATCTGTGGGAGAAACT
GTCACCATCACATGTCGAGCAAGTGAGAATATTTACAGTTATTTAGCATGGTATCAG
CAGAAACAGGGAAAATCTCCTCAGCTCCTGGTCTATAATGCAAAAACCTTAGCAGA
AGGTGTGCCATCAAGGTTCAGTGGCAGTGGATCAGGCACACAGTTTTCTCTGAAGAT
CAACAGCCTGCAGCCTGAAGATTTTGGGAGTTATTACTGTCAACATCATTATGGTTT
TCCGTGGACGTTCGGTGGAGGCACCAAGCTCGAGATCAAACGAACTGTGGCTGCAC
CATCTGTCTTCATCTTCCCGCCATCTGATGAGCAGTTGAAATCTGGAACTGCCTCTGT
TGTGTGCCTGCTGAATAACTTCTATCCCAGAGAGGCCAAAGTACAGTGGAAGGTGG
ATAACGCCCTCCAATCGGGTAACTCCCAGGAGAGTGTCACAGAGCAGGACAGCAAG
GACAGCACCTACAGCCTCAGCAGCACCCTGACGCTGAGCAAAGCAGACTACGAGAA
ACACAAAGTCTATGCCTGCGAAGTCACCCATCAGGGCCTGAGCTCGCCCGTCACAA
AGAGCTTCAACAGGGGAGAGTGTTAG
Anti-CD1d_2B5.3G10_K-V-hIgGK-C (SEQ ID NO: 79):
MSVPTQVLGLLLLWLTGARCDIQMAQSPASLSASVGETVTITCRASENIYSYLAWYQQK
QGKSPQLLVYNAKTLAEGVPSRFSGSGSGTQFSLKINSLQPEDFGSYYCQHHYGFPWTF
GGGTKLEIKRTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSFNRGEC
Anti-CD1d_2H11.2G5_H-V-hIgG4H-C (SEQ ID NO: 15):
ATGAACTTCGGGCTCAGCTTGATTTTCCTTGTCCTCATTTTAAAAGGTGTCCAGTGTG
AGGTGCAGCTGGTGGAGTCTGGGGGAGACTTAGTGAAGCCTGGAGGGTCCCTGAAA
CTCTCCTGTGCAGCCTCTGGATTCACTTTCAGTAGCTATGGCATGTCTTGGGTTCGCC
AGACTCCAGACAAGAGGCTGGAGTGGGTCGCAGTCATTAGTAGTGGTGGAAGTTCC
ACCTTCTATCCAGACAGTGTGAAGGGGCGATTCACCATCTCCAGAGACAATGCCAA
GAACACCCTGTACCTGCAAATGAGCAGTCTGAAGTCTGAGGACACAGCCGTGTATT
ACTGTTCAAGAGGAGGTTACTACTTTGACTACTGGGGCCAAGGCACCACTCTCACAG
TCTCCGCAGCCAAAACAAAGGGCCCATCCGTCTTCCCCCTGGCGCCCTGCTCCAGGA
GCACCTCCGAGAGCACAGCCGCCCTGGGCTGCCTGGTCAAGGACTACTTCCCCGAA
CCGGTGACGGTGTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCCCTC
CAGCAGCTTGGGCACGAAGACCTACACCTGCAACGTAGATCACAAGCCCAGCAACA
CCAAGGTGGACAAGAGAGTTGAGTCCAAATATGGTCCCCCATGCCCACCCTGCCCA
GCACCTGAGTTCGAAGGGGGACCATCAGTCTTCCTGTTCCCCCCAAAACCCAAGGAC
ACTCTCATGATCTCCCGGACCCCTGAGGTCACGTGCGTGGTGGTGGACGTGAGCCAG
GAAGACCCCGAGGTCCAGTTCAACTGGTACGTGGATGGCGTGGAGGTGCATAATGC
CAAGACAAAGCCGCGGGAGGAGCAGTTCAACAGCACGTACCGTGTGGTCAGCGTCC
TCACCGTCCTGCACCAGGACTGGCTGAACGGCAAGGAGTACAAGTGCAAGGTCTCC
AACAAAGGCCTCCCGTCCTCCATCGAGAAAACCATCTCCAAAGCCAAAGGGCAGCC
CCGAGAGCCACAGGTGTACACCCTGCCCCCATCCCAGGAGGAGATGACCAAGAACC
AGGTCAGCCTGACCTGCCTGGTCAAAGGCTTCTACCCCAGCGACATCGCCGTGGAGT
GGGAGAGCAATGGGCAGCCGGAGAACAACTACAAGACCACGCCTCCCGTGCTGGAC
TCCGACGGCTCCTTCTTCCTCTACAGCAGGCTAACCGTGGACAAGAGCAGGTGGCAG
GAGGGGAATGTCTTCTCATGCTCCGTGATGCATGAGGCTCTGCACAACCACTACACA
CAGAAGAGCCTCTCCCTGTCTCTGGGTAAAGCTAGCTGA
Anti-CD1d_2H11.2G5_H-V-hIgG4H-C (SEQ ID NO: 80):
MNFGLSLIFLVLILKGVQCEVQLVESGGDLVKPGGSLKLSCAASGFTFSSYGMSWVRQT
PDKRLEWVAVISSGGSSTFYPDSVKGRFTISRDNAKNTLYLQMSSLKSEDTAVYYCSRG
GYYFDYWGQGTTLTVSAAKTKGPSVFPLAPCSRSTSESTAALGCLVKDYFPEPVTVSWN
SGALTSGVHTFPAVLQSSGLYSLSSVVTVPSSSLGTKTYTCNVDHKPSNTKVDKRVESK
YGPPCPPCPAPEFEGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGLPSSIEKTISKAK
GQPREPQVYTLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGKAS
Anti-CD1d_2H11.2G5_K-V-hIgGK-C (SEQ ID NO: 16):
ATGAGGTTCCAGGTTCAGGTTCTGGGGCTCCTTCTGCTCTGGATATCAGGTGCCCAG
TGTGATGTCCAGATAACCCAGTCTCCATCTTATCTTGCTGCATCTCCTGGAGAAACC
ATTACTATTAATTGCAGGGCAAGCAAGACCATTAGCAAATATTTAGCCTGGTATCAA
GAGAAACCTGAGAAAACTGATAAGCTTCTTATCTACTCTGGATCCACTTTGCAATCT
GGAATTCCATCAAGGTTCAGTGGCAGTGGATCTGGTACAGATTTCACTCTCACCATC
AGTGGCCTGGAGCCTGAAGATTTTGCAATGTATTACTGTCAACAGCATAATGAATAC
CCGTGGACGTTCGGTGGAGGCACCAAGCTCGAGATCAAACGAACTGTGGCTGCACC
ATCTGTCTTCATCTTCCCGCCATCTGATGAGCAGTTGAAATCTGGAACTGCCTCTGTT
GTGTGCCTGCTGAATAACTTCTATCCCAGAGAGGCCAAAGTACAGTGGAAGGTGGA
TAACGCCCTCCAATCGGGTAACTCCCAGGAGAGTGTCACAGAGCAGGACAGCAAGG
ACAGCACCTACAGCCTCAGCAGCACCCTGACGCTGAGCAAAGCAGACTACGAGAAA
CACAAAGTCTATGCCTGCGAAGTCACCCATCAGGGCCTGAGCTCGCCCGTCACAAA
GAGCTTCAACAGGGGAGAGTGTTAG
Anti-CD1d_2H11.2G5_K-V-hIgGK-C (SEQ ID NO: 81):
MRFQVQVLGLLLLWISGAQCDVQITQSPSYLAASPGETITINCRASKTISKYLAWYQEKP
EKTDKLLIYSGSTLQSGIPSRFSGSGSGTDFTLTISGLEPEDFAMYYCQQHNEYPWTFGG
GTKLEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC
Anti-CD40_11B6.1C3_H-LV-hIgG4H-C (SEQ ID NO: 17):
ATGGGATGGAGCTGGATCTTTCTCTTTCTCCTGTCAGGAACTGCAGGTGTCCTCTCTG
AGGTCCAGCTGCAACAGTCTGGACCTGAGCTGGTGAAGCCTGGGGCTTCAGTGAAG
ATATCCTGCAAGGCTTCTGGTTACTCATTCACTGGCTACTACATGCACTGGGTGAAG
CAAAGCCATGTAAAGAGCCTTGAGTGGATTGGACGTATTAATCCTTACAATGGTGCT
ACTAGCTACAACCAGAATTTCAAGGACAAGGCCAGCTTGACTGTAGATAAGTCCTCC
AGCACAGCCTACATGGAGCTCCACAGCCTGACATCTGAGGACTCTGCAGTCTATTAC
TGTGCAAGAGAGGACTACGTCTACTGGGGCCAAGGCACCACTCTCACAGTCTCCTCA
GCCAAAACGAAGGGCCCATCCGTCTTCCCCCTGGCGCCCTGCTCCAGGAGCACCTCC
GAGAGCACAGCCGCCCTGGGCTGCCTGGTCAAGGACTACTTCCCCGAACCGGTGAC
GGTGTCGTGGAACTCAGGCGCCCTGACCAGCGGCGTGCACACCTTCCCGGCTGTCCT
ACAGTCCTCAGGACTCTACTCCCTCAGCAGCGTGGTGACCGTGCCCTCCAGCAGCTT
GGGCACGAAGACCTACACCTGCAACGTAGATCACAAGCCCAGCAACACCAAGGTGG
ACAAGAGAGTTGAGTCCAAATATGGTCCCCCATGCCCACCCTGCCCAGCACCTGAGT
TCGAAGGGGGACCATCAGTCTTCCTGTTCCCCCCAAAACCCAAGGACACTCTCATGA
TCTCCCGGACCCCTGAGGTCACGTGCGTGGTGGTGGACGTGAGCCAGGAAGACCCC
GAGGTCCAGTTCAACTGGTACGTGGATGGCGTGGAGGTGCATAATGCCAAGACAAA
GCCGCGGGAGGAGCAGTTCAACAGCACGTACCGTGTGGTCAGCGTCCTCACCGTCC
TGCACCAGGACTGGCTGAACGGCAAGGAGTACAAGTGCAAGGTCTCCAACAAAGGC
CTCCCGTCCTCCATCGAGAAAACCATCTCCAAAGCCAAAGGGCAGCCCCGAGAGCC
ACAGGTGTACACCCTGCCCCCATCCCAGGAGGAGATGACCAAGAACCAGGTCAGCC
TGACCTGCCTGGTCAAAGGCTTCTACCCCAGCGACATCGCCGTGGAGTGGGAGAGC
AATGGGCAGCCGGAGAACAACTACAAGACCACGCCTCCCGTGCTGGACTCCGACGG
CTCCTTCTTCCTCTACAGCAGGCTAACCGTGGACAAGAGCAGGTGGCAGGAGGGGA
ATGTCTTCTCATGCTCCGTGATGCATGAGGCTCTGCACAACCACTACACACAGAAGA
GCCTCTCCCTGTCTCTGGGTAAAGCTAGCTGA
Anti-CD40_11B6.1C3_H-LV-hIgG4H-C (SEQ ID NO: 82):
MGWSWIFLFLLSGTAGVLSEVQLQQSGPELVKPGASVKISCKASGYSFTGYYMHWVKQ
SHVKSLEWIGRINPYNGATSYNQNFKDKASLTVDKSSSTAYMELHSLTSEDSAVYYCAR
EDYVYWGQGTTLTVSSAKTKGPSVFPLAPCSRSTSESTAALGCLVKDYFPEPVTVSWNS
GALTSGVHTFPAVLQSSGLYSLSSVVTVPSSSLGTKTYTCNVDHKPSNTKVDKRVESKY
GPPCPPCPAPEFEGGPSVFLFPPKPKDTLMISRTPEVTCVVVDVSQEDPEVQFNWYVDGV
EVHNAKTKPREEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGKAS
Anti-CD40_11B6.1C3_K-LV-hIgGK-C (SEQ ID NO: 18):
ATGAAGTTGCCTGTTAGGCTGTTGGTGCTGATGTTCTGGATTCCTGCTTCCAGCAGTG
ATGTTGTGATGACCCAAACTCCACTCTCCCTGCCTGTCAGTCTTGGAGATCAAGCCT
CCATCTCTTGCAGATCTAGTCAGAGCCTTGTACACAGTAATGGAAACACCTATTTAC
ATTGGTACCTGCAGAAGCCAGGCCAGTCTCCAAAGCTCCTGATCTACAAAGTTTCCA
ACCGATTTTCTGGGGTCCCAGACAGGTTCAGTGGCAGTGGATCAGGGACAGATTTCG
CACTCAAGATCAGTAGAGTGGAGGCTGAGGATCTGGGAGTTTATTTCTGCTCTCAAA
GTACACATGTTCCGTGGACGTTCGGTGGAGGCACCAAGCTCGAGATCAAACGAACT
GTGGCTGCACCATCTGTCTTCATCTTCCCGCCATCTGATGAGCAGTTGAAATCTGGA
ACTGCCTCTGTTGTGTGCCTGCTGAATAACTTCTATCCCAGAGAGGCCAAAGTACAG
TGGAAGGTGGATAACGCCCTCCAATCGGGTAACTCCCAGGAGAGTGTCACAGAGCA
GGACAGCAAGGACAGCACCTACAGCCTCAGCAGCACCCTGACGCTGAGCAAAGCAG
ACTACGAGAAACACAAAGTCTATGCCTGCGAAGTCACCCATCAGGGCCTGAGCTCG
CCCGTCACAAAGAGCTTCAACAGGGGAGAGTGTTAG
Anti-CD40_11B6.1C3_K-LV-hIgGK-C (SEQ ID NO: 83):
MKLPVRLLVLMFWIPASSSDVVMTQTPLSLPVSLGDQASISCRSSQSLVHSNGNTYLHW
YLQKPGQSPKLLIYKVSNRFSGVPDRFSGSGSGTDFALKISRVEAEDLGVYFCSQSTHVP
WTFGGGTKLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
Anti-CD40_12B4.2C10_H-LV-hIgG4H-C (SEQ ID NO: 19):
ATGGAATGGAGTTGGATATTTCTCTTTCTTCTGTCAGGAACTGCAGGTGTCCACTCTG
AGGTCCAGCTGCAGCAGTCTGGACCTGAGCTGGTAAAGCCTGGGGCTTCAGTGAAG
ATGTCCTGCAAGGCTTCTGGATACACATTCACTGACTATGTTTTGCACTGGGTGAAA
CAGAAGCCTGGGCAGGGCCTTGAGTGGATTGGATATATTAATCCTTACAATGATGGT
ACTAAGTACAATGAGAAGTTCAAAGGCAAGGCCACACTGACTTCAGACAAATCCTC
CAGCACAGCCTACATGGAGCTCAGCAGCCTGACCTCTGAGGACTCTGCGGTCTATTA
CTGTGCAAGGGGCTATCCGGCCTACTCTGGGTATGCTATGGACTACTGGGGTCAAGG
AACCTCAGTCACCGTCTCCTCAGCCAAAACGAAGGGCCCATCCGTCTTCCCCCTGGC
GCCCTGCTCCAGGAGCACCTCCGAGAGCACAGCCGCCCTGGGCTGCCTGGTCAAGG
ACTACTTCCCCGAACCGGTGACGGTGTCGTGGAACTCAGGCGCCCTGACCAGCGGC
GTGCACACCTTCCCGGCTGTCCTACAGTCCTCAGGACTCTACTCCCTCAGCAGCGTG
GTGACCGTGCCCTCCAGCAGCTTGGGCACGAAGACCTACACCTGCAACGTAGATCA
CAAGCCCAGCAACACCAAGGTGGACAAGAGAGTTGAGTCCAAATATGGTCCCCCAT
GCCCACCCTGCCCAGCACCTGAGTTCGAAGGGGGACCATCAGTCTTCCTGTTCCCCC
CAAAACCCAAGGACACTCTCATGATCTCCCGGACCCCTGAGGTCACGTGCGTGGTG
GTGGACGTGAGCCAGGAAGACCCCGAGGTCCAGTTCAACTGGTACGTGGATGGCGT
GGAGGTGCATAATGCCAAGACAAAGCCGCGGGAGGAGCAGTTCAACAGCACGTAC
CGTGTGGTCAGCGTCCTCACCGTCCTGCACCAGGACTGGCTGAACGGCAAGGAGTA
CAAGTGCAAGGTCTCCAACAAAGGCCTCCCGTCCTCCATCGAGAAAACCATCTCCA
AAGCCAAAGGGCAGCCCCGAGAGCCACAGGTGTACACCCTGCCCCCATCCCAGGAG
GAGATGACCAAGAACCAGGTCAGCCTGACCTGCCTGGTCAAAGGCTTCTACCCCAG
CGACATCGCCGTGGAGTGGGAGAGCAATGGGCAGCCGGAGAACAACTACAAGACC
ACGCCTCCCGTGCTGGACTCCGACGGCTCCTTCTTCCTCTACAGCAGGCTAACCGTG
GACAAGAGCAGGTGGCAGGAGGGGAATGTCTTCTCATGCTCCGTGATGCATGAGGC
TCTGCACAACCACTACACACAGAAGAGCCTCTCCCTGTCTCTGGGTAAAGCTAGCTGA
Anti-CD40_12B4.2C10_H-LV-hIgG4H-C (SEQ ID NO: 84):
MEWSWIFLFLLSGTAGVHSEVQLQQSGPELVKPGASVKMSCKASGYTFTDYVLHWVK
QKPGQGLEWIGYINPYNDGTKYNEKFKGKATLTSDKSSSTAYMELSSLTSEDSAVYYCA
RGYPAYSGYAMDYWGQGTSVTVSSAKTKGPSVFPLAPCSRSTSESTAALGCLVKDYFP
EPVTVSWNSGALTSGVHTFPAVLQSSGLYSLSSVVTVPSSSLGTKTYTCNVDHKPSNTK
VDKRVESKYGPPCPPCPAPEFEGGPSVFLFPPKPKDTLMISRTPEVTCVVVDVSQEDPEV
QFNWYVDGVEVHNAKTKPREEQFNSTYRVVSVLTVLHQDWLNGKEYKCKVSNKGLPS
SIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGKAS
Anti-CD40_12B4.2C10_K-LV-v2-hIgGK-C (SEQ ID NO: 20):
ATGATGTCCTCTGCTCAGTTCCTTGGTCTCCTGTTGCTCTGTTTTCAAGGTACCAGAT
GTGATATCCAGATGACACAGACTACATCCTCCCTGTCTGCCTCTCTGGGAGACAGAG
TCACCATCAGTTGCAGGGCAAGTCAGGACATTAGCAATTATTTAAACTGGTATCAGC
AGAAACCAGATGGAACTGTTAAACTCCTGATCTACTACACATCAAGATTACACTCAG
GAGTCCCATCAAGGTTCAGTGGCAGTGGGTCTGGAACAGATTATTCTCTCACCATTA
GCAACCTGGAGCAAGAAGATATTGCCACTTACTTTTGCCATCATGGTAATACGCTTC
CGTGGACGTTCGGTGGAGGCACCAAGCTCGAGATCAAACGAACTGTGGCTGCACCA
TCTGTCTTCATCTTCCCGCCATCTGATGAGCAGTTGAAATCTGGAACTGCCTCTGTTG
TGTGCCTGCTGAATAACTTCTATCCCAGAGAGGCCAAAGTACAGTGGAAGGTGGAT
AACGCCCTCCAATCGGGTAACTCCCAGGAGAGTGTCACAGAGCAGGACAGCAAGGA
CAGCACCTACAGCCTCAGCAGCACCCTGACGCTGAGCAAAGCAGACTACGAGAAAC
ACAAAGTCTATGCCTGCGAAGTCACCCATCAGGGCCTGAGCTCGCCCGTCACAAAG
AGCTTCAACAGGGGAGAGTGTTAG
Anti-CD40_12B4.2C10_K-LV-v2-hIgGK-C (SEQ ID NO: 85):
MMSSAQFLGLLLLCFQGTRCDIQMTQTTSSLSASLGDRVTISCRASQDISNYLNWYQQK
PDGTVKLLIYYTSRLHSGVPSRFSGSGSGTDYSLTISNLEQEDIATYFCHHGNTLPWTFGG
GTKLEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC
Anti-CD40_12E12.3F3_H-V-hIgG4H-C (SEQ ID NO: 21):
ATGAACTTGGGGCTCAGCTTGATTTTCCTTGTCCTTGTTTTAAAAGGTGTCCAGTGTG
AAGTGAAGCTGGTGGAGTCTGGGGGAGGCTTAGTGCAGCCTGGAGGGTCCCTGAAA
CTCTCCTGTGCAACCTCTGGATTCACTTTCAGTGACTATTACATGTATTGGGTTCGCC
AGACTCCAGAGAAGAGGCTGGAGTGGGTCGCATACATTAATTCTGGTGGTGGTAGC
ACCTATTATCCAGACACTGTAAAGGGCCGATTCACCATCTCCAGAGACAATGCCAA
GAACACCCTGTACCTGCAAATGAGCCGGCTGAAGTCTGAGGACACAGCCATGTATT
ACTGTGCAAGACGGGGGTTACCGTTCCATGCTATGGACTATTGGGGTCAAGGAACCT
CAGTCACCGTCTCCTCAGCCAAAACGAAGGGCCCATCCGTCTTCCCCCTGGCGCCCT
GCTCCAGGAGCACCTCCGAGAGCACAGCCGCCCTGGGCTGCCTGGTCAAGGACTAC
TTCCCCGAACCGGTGACGGTGTCGTGGAACTCAGGCGCCCTGACCAGCGGCGTGCA
CACCTTCCCGGCTGTCCTACAGTCCTCAGGACTCTACTCCCTCAGCAGCGTGGTGAC
CGTGCCCTCCAGCAGCTTGGGCACGAAGACCTACACCTGCAACGTAGATCACAAGC
CCAGCAACACCAAGGTGGACAAGAGAGTTGAGTCCAAATATGGTCCCCCATGCCCA
CCCTGCCCAGCACCTGAGTTCGAAGGGGGACCATCAGTCTTCCTGTTCCCCCCAAAA
CCCAAGGACACTCTCATGATCTCCCGGACCCCTGAGGTCACGTGCGTGGTGGTGGAC
GTGAGCCAGGAAGACCCCGAGGTCCAGTTCAACTGGTACGTGGATGGCGTGGAGGT
GCATAATGCCAAGACAAAGCCGCGGGAGGAGCAGTTCAACAGCACGTACCGTGTGG
TCAGCGTCCTCACCGTCCTGCACCAGGACTGGCTGAACGGCAAGGAGTACAAGTGC
AAGGTCTCCAACAAAGGCCTCCCGTCCTCCATCGAGAAAACCATCTCCAAAGCCAA
AGGGCAGCCCCGAGAGCCACAGGTGTACACCCTGCCCCCATCCCAGGAGGAGATGA
CCAAGAACCAGGTCAGCCTGACCTGCCTGGTCAAAGGCTTCTACCCCAGCGACATC
GCCGTGGAGTGGGAGAGCAATGGGCAGCCGGAGAACAACTACAAGACCACGCCTC
CCGTGCTGGACTCCGACGGCTCCTTCTTCCTCTACAGCAGGCTAACCGTGGACAAGA
GCAGGTGGCAGGAGGGGAATGTCTTCTCATGCTCCGTGATGCATGAGGCTCTGCACA
ACCACTACACACAGAAGAGCCTCTCCCTGTCTCTGGGTAAAGCTAGCTGA
Anti-CD40_12E12.3F3_H-V-hIgG4H-C (SEQ ID NO: 86):
MNLGLSLIFLVLVLKGVQCEVKLVESGGGLVQPGGSLKLSCATSGFTFSDYYMYWVRQ
TPEKRLEWVAYINSGGGSTYYPDTVKGRFTISRDNAKNTLYLQMSRLKSEDTAMYYCA
RRGLPFHAMDYWGQGTSVTVSSAKTKGPSVFPLAPCSRSTSESTAALGCLVKDYFPEPV
TVSWNSGALTSGVHTFPAVLQSSGLYSLSSVVTVPSSSLGTKTYTCNVDHKPSNTKVDK
RVESKYGPPCPPCPAPEFEGGPSVFLFPPKPKDTLMISRTPEVTCVVVDVSQEDPEVQFN
WYVDGVEVHNAKTKPREEQFNSTYRVVSVLTVLHQDWLNGKEYKCKVSNKGLPSSIE
KTISKAKGQPREPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSLSLSLGKAS
Anti-CD40_12E12.3F3_K-LV-hIgGK-C (SEQ ID NO: 22):
ATGATGTCCTCTGCTCAGTTCCTTGGTCTCCTGTTGCTCTGTTTTCAAGGTACCAGAT
GTGATATCCAGATGACACAGACTACATCCTCCCTGTCTGCCTCTCTAGGAGACAGAG
TCACCATCAGTTGCAGTGCAAGTCAGGGCATTAGCAATTATTTAAACTGGTATCAGC
AGAAACCAGATGGAACTGTTAAACTCCTGATCTATTACACATCAATTTTACACTCAG
GAGTCCCATCAAGGTTCAGTGGCAGTGGGTCTGGGACAGATTATTCTCTCACCATCG
GCAACCTGGAACCTGAAGATATTGCCACTTACTATTGTCAGCAGTTTAATAAGCTTC
CTCCGACGTTCGGTGGAGGCACCAAACTCGAGATCAAACGAACTGTGGCTGCACCA
TCTGTCTTCATCTTCCCGCCATCTGATGAGCAGTTGAAATCTGGAACTGCCTCTGTTG
TGTGCCTGCTGAATAACTTCTATCCCAGAGAGGCCAAAGTACAGTGGAAGGTGGAT
AACGCCCTCCAATCGGGTAACTCCCAGGAGAGTGTCACAGAGCAGGACAGCAAGGA
CAGCACCTACAGCCTCAGCAGCACCCTGACGCTGAGCAAAGCAGACTACGAGAAAC
ACAAAGTCTATGCCTGCGAAGTCACCCATCAGGGCCTGAGCTCGCCCGTCACAAAG
AGCTTCAACAGGGGAGAGTGTTAG
Anti-CD40_12E12.3F3_K-LV-hIgGK-C (SEQ ID NO: 87):
MMSSAQFLGLLLLCFQGTRCDIQMTQTTSSLSASLGDRVTISCSASQGISNYLNWYQQK
PDGTVKLLIYYTSILHSGVPSRFSGSGSGTDYSLTIGNLEPEDIATYYCQQFNKLPPTFGG
GTKLEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC
Anti-DCIR_24A5.4A5_H-V-hIgG4H-C (SEQ ID NO: 23):
ATGGATTGGCTGTGGAACTTGCTATTCCTGATGGCAGCTGCCCAAAGTGCCCAAGCA
CAGATCCAGTTGGTGCAGTCTGGACCTGAGCTGAAGAAGCCTGGAGAGACAGTCAA
GATCTCCTGCAAGGCTTCTGGGTATTCCTTCACAAACTATGGAATGAACTGGGTGAA
ACAGGCTCCAGGAAAGGGTTTAAAGTGGATGGGCTGGATAAACACCTACACTGGAG
AGTCAACATATGCTGATGACTTCAAGGGACGGTTTGCCTTCTCTTTGGAAACCTCTG
CCAGCACTGCCTATTTGCAGATCAGTAACCTCAAAAATGAGGACATGGCTACATATT
TCTGTGCTAGAGGGGACTTTAGGTACTACTATTTTGACTACTGGGGCCAAGGCACCA
CTCTCACAGGCTCCTCAGCCAAAACGAAGGGCCCATCCGTCTTCCCCCTGGCGCCCT
GCTCCAGGAGCACCTCCGAGAGCACAGCCGCCCTGGGCTGCCTGGTCAAGGACTAC
TTCCCCGAACCGGTGACGGTGTCGTGGAACTCAGGCGCCCTGACCAGCGGCGTGCA
CACCTTCCCGGCTGTCCTACAGTCCTCAGGACTCTACTCCCTCAGCAGCGTGGTGAC
CGTGCCCTCCAGCAGCTTGGGCACGAAGACCTACACCTGCAACGTAGATCACAAGC
CCAGCAACACCAAGGTGGACAAGAGAGTTGAGTCCAAATATGGTCCCCCATGCCCA
CCCTGCCCAGCACCTGAGTTCGAAGGGGGACCATCAGTCTTCCTGTTCCCCCCAAAA
CCCAAGGACACTCTCATGATCTCCCGGACCCCTGAGGTCACGTGCGTGGTGGTGGAC
GTGAGCCAGGAAGACCCCGAGGTCCAGTTCAACTGGTACGTGGATGGCGTGGAGGT
GCATAATGCCAAGACAAAGCCGCGGGAGGAGCAGTTCAACAGCACGTACCGTGTGG
TCAGCGTCCTCACCGTCCTGCACCAGGACTGGCTGAACGGCAAGGAGTACAAGTGC
AAGGTCTCCAACAAAGGCCTCCCGTCCTCCATCGAGAAAACCATCTCCAAAGCCAA
AGGGCAGCCCCGAGAGCCACAGGTGTACACCCTGCCCCCATCCCAGGAGGAGATGA
CCAAGAACCAGGTCAGCCTGACCTGCCTGGTCAAAGGCTTCTACCCCAGCGACATC
GCCGTGGAGTGGGAGAGCAATGGGCAGCCGGAGAACAACTACAAGACCACGCCTC
CCGTGCTGGACTCCGACGGCTCCTTCTTCCTCTACAGCAGGCTAACCGTGGACAAGA
GCAGGTGGCAGGAGGGGAATGTCTTCTCATGCTCCGTGATGCATGAGGCTCTGCACA
ACCACTACACACAGAAGAGCCTCTCCCTGTCTCTGGGTAAAGCTAGCTGAT
Anti-DCIR_24A5.4A5_H-V-hIgG4H-C (SEQ ID NO: 88):
MDWLWNLLFLMAAAQSAQAQIQLVQSGPELKKPGETVKISCKASGYSFTNYGMNWVK
QAPGKGLKWMGWINTYTGESTYADDFKGRFAFSLETSASTAYLQISNLKNEDMATYFC
ARGDFRYYYFDYWGQGTTLTGSSAKTKGPSVFPLAPCSRSTSESTAALGCLVKDYFPEP
VTVSWNSGALTSGVHTFPAVLQSSGLYSLSSVVTVPSSSLGTKTYTCNVDHKPSNTKVD
KRVESKYGPPCPPCPAPEFEGGPSVFLFPPKPKDTLMISRTPEVTCVVVDVSQEDPEVQF
NWYVDGVEVHNAKTKPREEQFNSTYRVVSVLTVLHQDWLNGKEYKCKVSNKGLPSSI
EKTISKAKGQPREPQVYTLPPSQEEMTKNQVSLTCLVKGFYPSDIAVEWESNGQPENNY
KTTPPVLDSDGSFFLYSRLTVDKSRWQEGNVFSCSVMHEALHNHYTQKSLSLSLGKAS
Anti-DCIR_24A5.4A5_K-V-hIgGK-C (SEQ ID NO: 24):
ATGAGTGTGCTCACTCAGGTCCTGGCGTTGCTGCTGCTGTGGCTTACAGGTGCCAGA
TGTGACATCCAGATGACTCAGTCTCCAGCCTCCCTATCTGCATCTGTGGGAGAAACT
GTCACCATCACGTGTCGAGCAAGTGGGAATATTCACAATTATTTAGCATGGTATCAG
CAGAAACAGGGAAAATCTCCTCAGCTCCTGGTCTATAATGCAAAAACCTTGGCAGA
TGGTGTGCCATCAAGGTTCAGTGGCAGTGGATCAGGAACACAATATTCTCTCAAGAT
CAACACCCTGCAGCCTGAAGATTTTGGGAGTTATTACTGTCAACATTTTTGGGATTCT
TGGACGTTCGGTGGAGGCACCAAGCTCGAGATCAAACGAACTGTGGCTGCACCATC
TGTCTTCATCTTCCCGCCATCTGATGAGCAGTTGAAATCTGGAACTGCCTCTGTTGTG
TGCCTGCTGAATAACTTCTATCCCAGAGAGGCCAAAGTACAGTGGAAGGTGGATAA
CGCCCTCCAATCGGGTAACTCCCAGGAGAGTGTCACAGAGCAGGACAGCAAGGACA
GCACCTACAGCCTCAGCAGCACCCTGACGCTGAGCAAAGCAGACTACGAGAAACAC
AAAGTCTATGCCTGCGAAGTCACCCATCAGGGCCTGAGCTCGCCCGTCACAAAGAG
CTTCAACAGGGGAGAGTGTTAG
Anti-DCIR_24A5.4A5_K-V-hIgGK-C (SEQ ID NO: 89):
MSVLTQVLALLLLWLTGARCDIQMTQSPASLSASVGETVTITCRASGNIHNYLAWYQQ
KQGKSPQLLVYNAKTLADGVPSRFSGSGSGTQYSLKINTLQPEDFGSYYCQHFWDSWTF
GGGTKLEIKRTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSFNRGEC
Anti-DCIR_24E7.3H9_H-V-hIgG4H-C (SEQ ID NO: 25):
ATGGAATGGACCTGGGTCTTTCTCTTCCTCCTGTCAGTAACTGCAGGTGTCCACTCCC
AGGTTCAGCTGCAGCAGTCTGGAGCTGAGCTGATGAAGCCTGGGGCCTCAGTGAAG
ATATCCTGCAAGGCTACTGGCTACACATTCAGTAGCTACTGGATAGAGTGGGTAAAG
CAGAGGCCTGGACATGGCCTTGAGTGGATTGGAGAGATTTTACCTGGAAGTGGTAG
GACTAACGACAATGAGAAGTTCAAGGGCAAGGCCACATTCACTGCAGATACATCCT
CCAAGAAAGCCTACATGCAACTCAGCAGCCTGACATCTGAGGACTCTGCCGTCTATT
ATTGTGCAAGAAGGGGTGGTTACTCCTTTGCTTACTGGGGCCAAGGGACTCTGGTCA
CTGTCTCTGCAGCCAAAACAAAGGGCCCATCCGTCTTCCCCCTGGCGCCCTGCTCCA
GGAGCACCTCCGAGAGCACAGCCGCCCTGGGCTGCCTGGTCAAGGACTACTTCCCC
GAACCGGTGACGGTGTCGTGGAACTCAGGCGCCCTGACCAGCGGCGTGCACACCTT
CCCGGCTGTCCTACAGTCCTCAGGACTCTACTCCCTCAGCAGCGTGGTGACCGTGCC
CTCCAGCAGCTTGGGCACGAAGACCTACACCTGCAACGTAGATCACAAGCCCAGCA
ACACCAAGGTGGACAAGAGAGTTGAGTCCAAATATGGTCCCCCATGCCCACCCTGC
CCAGCACCTGAGTTCGAAGGGGGACCATCAGTCTTCCTGTTCCCCCCAAAACCCAAG
GACACTCTCATGATCTCCCGGACCCCTGAGGTCACGTGCGTGGTGGTGGACGTGAGC
CAGGAAGACCCCGAGGTCCAGTTCAACTGGTACGTGGATGGCGTGGAGGTGCATAA
TGCCAAGACAAAGCCGCGGGAGGAGCAGTTCAACAGCACGTACCGTGTGGTCAGCG
TCCTCACCGTCCTGCACCAGGACTGGCTGAACGGCAAGGAGTACAAGTGCAAGGTC
TCCAACAAAGGCCTCCCGTCCTCCATCGAGAAAACCATCTCCAAAGCCAAAGGGCA
GCCCCGAGAGCCACAGGTGTACACCCTGCCCCCATCCCAGGAGGAGATGACCAAGA
ACCAGGTCAGCCTGACCTGCCTGGTCAAAGGCTTCTACCCCAGCGACATCGCCGTGG
AGTGGGAGAGCAATGGGCAGCCGGAGAACAACTACAAGACCACGCCTCCCGTGCTG
GACTCCGACGGCTCCTTCTTCCTCTACAGCAGGCTAACCGTGGACAAGAGCAGGTGG
CAGGAGGGGAATGTCTTCTCATGCTCCGTGATGCATGAGGCTCTGCACAACCACTAC
ACACAGAAGAGCCTCTCCCTGTCTCTGGGTAAAGCTAGCTGA
Anti-DCIR_24E7.3H9_H-V-hIgG4H-C (SEQ ID NO: 90):
MEWTWVFLFLLSVTAGVHSQVQLQQSGAELMKPGASVKISCKATGYTFSSYWIEWVK
QRPGHGLEWIGEILPGSGRTNDNEKFKGKATFTADTSSKKAYMQLSSLTSEDSAVYYCA
RRGGYSFAYWGQGTLVTVSAAKTKGPSVFPLAPCSRSTSESTAALGCLVKDYFPEPVTV
SWNSGALTSGVHTFPAVLQSSGLYSLSSVVTVPSSSLGTKTYTCNVDHKPSNTKVDKRV
ESKYGPPCPPCPAPEFEGGPSVFLFPPKPKDTLMISRTPEVTCVVVDVSQEDPEVQFNWY
VDGVEVHNAKTKPREEQFNSTYRVVSVLTVLHQDWLNGKEYKCKVSNKGLPSSIEKTIS
KAKGQPREPQVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSLGKAS
Anti-DCIR_24E7.3H9_K-V-hIgGK-C (SEQ ID NO: 26):
ATGACCATGTTCTCACTAGCTCTTCTCCTCAGTCTTCTTCTCCTCTGTGTCTCTGATTC
TAGGGCAGAAACAACTGTGACCCAGTCTATGACCATGTTCTCACTAGCTCTTCTCCT
CAGTCTTCTTCTCCTCTGTGTCTCTGATTCTAGGGCAGAAACAACTGTGACCCAGTCT
CCAGCATCCCTGTCCATGGCTATAGGGGAAAAAGTCACCATCAGATGCGTAACCAG
CACTGATATTGATGATGATGTGAACTGGTACCAGCAGAAGCCAGGGGAACCTCCTA
AACTCCTTATTTCAGAAGGCAATACTCTTCGTCCTGGAGTCCCATCCCGATTCTCCAG
CAGTGGCTATGGTACAGATTTTGTTTTTACAATTGAGAACATGCTCTCAGAAGATGT
TGCAGATTACTACTGTTTGCAAAGTGGTAACTTGCCGTACACGTTCGGAGGGGGGAC
CAAGCTCGAGATCAAACGAACTGTGGCTGCACCATCTGTCTTCATCTTCCCGCCATC
TGATGAGCAGTTGAAATCTGGAACTGCCTCTGTTGTGTGCCTGCTGAATAACTTCTA
TCCCAGAGAGGCCAAAGTACAGTGGAAGGTGGATAACGCCCTCCAATCGGGTAACT
CCCAGGAGAGTGTCACAGAGCAGGACAGCAAGGACAGCACCTACAGCCTCAGCAG
CACCCTGACGCTGAGCAAAGCAGACTACGAGAAACACAAAGTCTATGCCTGCGAAG
TCACCCATCAGGGCCTGAGCTCGCCCGTCACAAAGAGCTTCAACAGGGGAGAGTGT
TAGCCAGCATCCCTGTCCATGGCTATAGGGGAAAAAGTCACCATCAGATGCGTAAC
CAGCACTGATATTGATGATGATGTGAACTGGTACCAGCAGAAGCCAGGGGAACCTC
CTAAACTCCTTATTTCAGAAGGCAATACTCTTCGTCCTGGAGTCCCATCCCGATTCTC
CAGCAGTGGCTATGGTACAGATTTTGTTTTTACAATTGAGAACATGCTCTCAGAAGA
TGTTGCAGATTACTACTGTTTGCAAAGTGGTAACTTGCCGTACACGTTCGGAGGGGG
GACCAAGCTCGAGATCAAACGAACTGTGGCTGCACCATCTGTCTTCATCTTCCCGCC
ATCTGATGAGCAGTTGAAATCTGGAACTGCCTCTGTTGTGTGCCTGCTGAATAACTT
CTATCCCAGAGAGGCCAAAGTACAGTGGAAGGTGGATAACGCCCTCCAATCGGGTA
ACTCCCAGGAGAGTGTCACAGAGCAGGACAGCAAGGACAGCACCTACAGCCTCAGC
AGCACCCTGACGCTGAGCAAAGCAGACTACGAGAAACACAAAGTCTATGCCTGCGA
AGTCACCCATCAGGGCCTGAGCTCGCCCGTCACAAAGAGCTTCAACAGGGGAGAGT
GTTAG
Anti-DCIR_24E7.3H9_K-V-hIgGK-C (SEQ ID NO: 91):
MTMFSLALLLSLLLLCVSDSRAETTVTQSPASLSMAIGEKVTIRCVTSTDIDDDVNWYQ
QKPGEPPKLLISEGNTLRPGVPSRFSSSGYGTDFVFTIENMLSEDVADYYCLQSGNLPYTF
GGGTKLEIKRTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSFNRGEC
Anti-DCIR_29E9.2E2_H-VhIgG4H-C (SEQ ID NO: 27):
ATGGCTTGGGTGTGGACCTTGCTATTCCTGATGGCAGCTGCCCAAAGTGCCCAAGCA
CAGATCCAGTTGGTGCAGTCTGGACCTGAGCTGAAGAAGCCTGGAGAGACAGTCAA
GATCTCCTGCAAGGCTTCTGGGTATACCTTCACAAACTATGGAATGAACTGGGTGAA
GCAGGCTCCAGGAAAGGGTTTAAAGTGGGTGGGCTGGATAAACACCTTCACTGGAG
AGCCAACATATGTTGATGACTTCAAGGGACGGTTTGCCTTCTCTTTGGAAACCTCTG
CCAGCACTGCCTATTTGCAGATCAACAACCTCAAAAATGAGGACACGGCTACATATT
TCTGTGCAAGAGGGAATTTTAGGTACTACTACTTTGACTACTGGGGCCAAGGCACCA
CTCTCACAGTCTCCTCAGCCAAAACAAAGGGCCCATCCGTCTTCCCCCTGGCGCCCT
GCTCCAGGAGCACCTCCGAGAGCACAGCCGCCCTGGGCTGCCTGGTCAAGGACTAC
TTCCCCGAACCGGTGACGGTGTCGTGGAACTCAGGCGCCCTGACCAGCGGCGTGCA
CACCTTCCCGGCTGTCCTACAGTCCTCAGGACTCTACTCCCTCAGCAGCGTGGTGAC
CGTGCCCTCCAGCAGCTTGGGCACGAAGACCTACACCTGCAACGTAGATCACAAGC
CCAGCAACACCAAGGTGGACAAGAGAGTTGAGTCCAAATATGGTCCCCCATGCCCA
CCCTGCCCAGCACCTGAGTTCGAAGGGGGACCATCAGTCTTCCTGTTCCCCCCAAAA
CCCAAGGACACTCTCATGATCTCCCGGACCCCTGAGGTCACGTGCGTGGTGGTGGAC
GTGAGCCAGGAAGACCCCGAGGTCCAGTTCAACTGGTACGTGGATGGCGTGGAGGT
GCATAATGCCAAGACAAAGCCGCGGGAGGAGCAGTTCAACAGCACGTACCGTGTGG
TCAGCGTCCTCACCGTCCTGCACCAGGACTGGCTGAACGGCAAGGAGTACAAGTGC
AAGGTCTCCAACAAAGGCCTCCCGTCCTCCATCGAGAAAACCATCTCCAAAGCCAA
AGGGCAGCCCCGAGAGCCACAGGTGTACACCCTGCCCCCATCCCAGGAGGAGATGA
CCAAGAACCAGGTCAGCCTGACCTGCCTGGTCAAAGGCTTCTACCCCAGCGACATC
GCCGTGGAGTGGGAGAGCAATGGGCAGCCGGAGAACAACTACAAGACCACGCCTC
CCGTGCTGGACTCCGACGGCTCCTTCTTCCTCTACAGCAGGCTAACCGTGGACAAGA
GCAGGTGGCAGGAGGGGAATGTCTTCTCATGCTCCGTGATGCATGAGGCTCTGCACA
ACCACTACACACAGAAGAGCCTCTCCCTGTCTCTGGGTAAAGCTAGCTGA
Anti-DCIR_29E9.2E2_H-VhIgG4H-C (SEQ ID NO: 92):
MAWVWTLLFLMAAAQSAQAQIQLVQSGPELKKPGETVKISCKASGYTFTNYGMNWVK
QAPGKGLKWVGWINTFTGEPTYVDDFKGRFAFSLETSASTAYLQINNLKNEDTATYFCA
RGNFRYYYFDYWGQGTTLTVSSAKTKGPSVFPLAPCSRSTSESTAALGCLVKDYFPEPV
TVSWNSGALTSGVHTFPAVLQSSGLYSLSSVVTVPSSSLGTKTYTCNVDHKPSNTKVDK
RVESKYGPPCPPCPAPEFEGGPSVFLFPPKPKDTLMISRTPEVTCVVVDVSQEDPEVQFN
WYVDGVEVHNAKTKPREEQFNSTYRVVSVLTVLHQDWLNGKEYKCKVSNKGLPSSIE
KTISKAKGQPREPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSLSLSLGKAS
Anti-DCIR_29E9.2E2_K-V-hIgGK-C (SEQ ID NO: 28):
ATGAGTGTGCTCACTCAGGTCCTGGCGTTGCTGCTGCTGTGGCTTACAGGTGCCAGA
TGTGACATCCAGATGACTCAGTCCCCAGCCTCCCTATCTGCATCTGTGGGAGAAACT
GTCACCATCACATGTCGAACAAGTGGGAATATTCGCAATTATTTAGCATGGTATCAG
CAGAAACAGGGAAAATCTCCTCAACTCCTGGTCTATAATGCAAAAACCTTAGCAGA
TGGTGTGCCATCAAGGTTCGGTGGCAGTGGATCAGGAACACAATATTCTCTCAAGAT
CAACAGCCTGCAGCCTGAAGATTTTGGGAATTATTACTGTCAACATTTTTGGAGTAG
TCCGTACACGTTCGGAGGGGGGACCAAGCTCGAGATCAAACGAACTGTGGCTGCAC
CATCTGTCTTCATCTTCCCGCCATCTGATGAGCAGTTGAAATCTGGAACTGCCTCTGT
TGTGTGCCTGCTGAATAACTTCTATCCCAGAGAGGCCAAAGTACAGTGGAAGGTGG
ATAACGCCCTCCAATCGGGTAACTCCCAGGAGAGTGTCACAGAGCAGGACAGCAAG
GACAGCACCTACAGCCTCAGCAGCACCCTGACGCTGAGCAAAGCAGACTACGAGAA
ACACAAAGTCTATGCCTGCGAAGTCACCCATCAGGGCCTGAGCTCGCCCGTCACAA
AGAGCTTCAACAGGGGAGAGTGTTAG
Anti-DCIR_29E9.2E2_K-V-hIgGK-C (SEQ ID NO: 93):
MSVLTQVLALLLLWLTGARCDIQMTQSPASLSASVGETVTITCRTSGNIRNYLAWYQQK
QGKSPQLLVYNAKTLADGVPSRFGGSGSGTQYSLKINSLQPEDFGNYYCQHFWSSPYTF
GGGTKLEIKRTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSFNRGEC
Anti-DCIR_29G10.3D9_H-V-hIgG4H-C (SEQ ID NO: 29):
ATGATGGGATGGAGCTATATCATCCTCTTTTTGGTAGCAACAGCTACAGATGTCCAC
TCCCAGGTCCAACTGCAGCAGCCTGGGGCTGAACTGGTGAAGCCTGGGGCTTCAGT
GAAGCTGTCCTGCAAGGCTTCTGGCTACACCTTCACCAGCTACTGGATGCACTGGGT
GAAGCAGAGGCCTGGAGAAGGCCTTGAGTGGATTGGAGAGATTAATCCTAGCTACG
GTCGTACTGACTACAATGAGAAGTTCAAGAACAAGGCCACACTGACTGTAGCCAAA
TCCTCCAGCACAGCCTACATGCAACTCAGCAGCCTGACATCTGAGGACTCTGCGGTC
TATTACTGTGCAAGAGGAGATTACTACGGTAGTAGCTCGTTTGCTTACTGGGGCCAA
GGGACTCTGGTCACTGTCTCTGCAGCCAAAACAAAGGGCCCATCCGTCTTCCCCCTG
GCGCCCTGCTCCAGGAGCACCTCCGAGAGCACAGCCGCCCTGGGCTGCCTGGTCAA
GGACTACTTCCCCGAACCGGTGACGGTGTCGTGGAACTCAGGCGCCCTGACCAGCG
GCGTGCACACCTTCCCGGCTGTCCTACAGTCCTCAGGACTCTACTCCCTCAGCAGCG
TGGTGACCGTGCCCTCCAGCAGCTTGGGCACGAAGACCTACACCTGCAACGTAGAT
CACAAGCCCAGCAACACCAAGGTGGACAAGAGAGTTGAGTCCAAATATGGTCCCCC
ATGCCCACCCTGCCCAGCACCTGAGTTCGAAGGGGGACCATCAGTCTTCCTGTTCCC
CCCAAAACCCAAGGACACTCTCATGATCTCCCGGACCCCTGAGGTCACGTGCGTGGT
GGTGGACGTGAGCCAGGAAGACCCCGAGGTCCAGTTCAACTGGTACGTGGATGGCG
TGGAGGTGCATAATGCCAAGACAAAGCCGCGGGAGGAGCAGTTCAACAGCACGTAC
CGTGTGGTCAGCGTCCTCACCGTCCTGCACCAGGACTGGCTGAACGGCAAGGAGTA
CAAGTGCAAGGTCTCCAACAAAGGCCTCCCGTCCTCCATCGAGAAAACCATCTCCA
AAGCCAAAGGGCAGCCCCGAGAGCCACAGGTGTACACCCTGCCCCCATCCCAGGAG
GAGATGACCAAGAACCAGGTCAGCCTGACCTGCCTGGTCAAAGGCTTCTACCCCAG
CGACATCGCCGTGGAGTGGGAGAGCAATGGGCAGCCGGAGAACAACTACAAGACC
ACGCCTCCCGTGCTGGACTCCGACGGCTCCTTCTTCCTCTACAGCAGGCTAACCGTG
GACAAGAGCAGGTGGCAGGAGGGGAATGTCTTCTCATGCTCCGTGATGCATGAGGC
TCTGCACAACCACTACACACAGAAGAGCCTCTCCCTGTCTCTGGGTAAAGCTAGCGG
ATGGAGCTATATCATCCTCTTTTTGGTAGCAACAGCTACAGATGTCCACTCCCAGGT
CCAACTGCAGCAGCCTGGGGCTGAACTGGTGAAGCCTGGGGCTTCAGTGAAGCTGT
CCTGCAAGGCTTCTGGCTACACCTTCACCAGCTACTGGATGCACTGGGTGAAGCAGA
GGCCTGGAGAAGGCCTTGAGTGGATTGGAGAGATTAATCCTAGCTACGGTCGTACT
GACTACAATGGGAAGTTCAAGAACAAGGCCACACTGACTGTAGCCAAATCCTCCAG
CACAGCCTACATGCAACTCAGCAGCCTGACATCTGAGGACTCTGCGGTCTATTACTG
TGCAAGAGGAGATTACTACGGTAGTAGCTCGTTTGCTTACTGGGGCCAAGGGACTCT
GGTCACTGTCTCTGCAGCCAAAACAAAGGGCCCATCCGTCTTCCCCCTGGCGCCCTG
CTCCAGGAGCACCTCCGAGAGCACAGCCGCCCTGGGCTGCCTGGTCAAGGACTACT
TCCCCGAACCGGTGACGGTGTCGTGGAACTCAGGCGCCCTGACCAGCGGCGTGCAC
ACCTTCCCGGCTGTCCTACAGTCCTCAGGACTCTACTCCCTCAGCAGCGTGGTGACC
GTGCCCTCCAGCAGCTTGGGCACGAAGACCTACACCTGCAACGTAGATCACAAGCC
CAGCAACACCAAGGTGGACAAGAGAGTTGAGTCCAAATATGGTCCCCCATGCCCAC
CCTGCCCAGCACCTGAGTTCGAAGGGGGACCATCAGTCTTCCTGTTCCCCCCAAAAC
CCAAGGACACTCTCATGATCTCCCGGACCCCTGAGGTCACGTGCGTGGTGGTGGACG
TGAGCCAGGAAGACCCCGAGGTCCAGTTCAACTGGTACGTGGATGGCGTGGAGGTG
CATAATGCCAAGACAAAGCCGCGGGAGGAGCAGTTCAACAGCACGTACCGTGTGGT
CAGCGTCCTCACCGTCCTGCACCAGGACTGGCTGAACGGCAAGGAGTACAAGTGCA
AGGTCTCCAACAAAGGCCTCCCGTCCTCCATCGAGAAAACCATCTCCAAAGCCAAA
GGGCAGCCCCGAGAGCCACAGGTGTACACCCTGCCCCCATCCCAGGAGGAGATGAC
CAAGAACCAGGTCAGCCTGACCTGCCTGGTCAAAGGCTTCTACCCCAGCGACATCG
CCGTGGAGTGGGAGAGCAATGGGCAGCCGGAGAACAACTACAAGACCACGCCTCCC
GTGCTGGACTCCGACGGCTCCTTCTTCCTCTACAGCAGGCTAACCGTGGACAAGAGC
AGGTGGCAGGAGGGGAATGTCTTCTCATGCTCCGTGATGCATGAGGCTCTGCACAAC
CACTACACACAGAAGAGCCTCTCCCTGTCTCTGGGTAAAGCTAGCTGA
Anti-DCIR_29G10.3D9_H-V-hIgG4H-C (SEQ ID NO: 94):
MMGWSYIILFLVATATDVHSQVQLQQPGAELVKPGASVKLSCKASGYTFTSYWMHWV
KQRPGEGLEWIGEINPSYGRTDYNEKFKNKATLTVAKSSSTAYMQLSSLTSEDSAVYYC
ARGDYYGSSSFAYWGQGTLVTVSAAKTKGPSVFPLAPCSRSTSESTAALGCLVKDYFPE
PVTVSWNSGALTSGVHTFPAVLQSSGLYSLSSVVTVPSSSLGTKTYTCNVDHKPSNTKV
DKRVESKYGPPCPPCPAPEFEGGPSVFLFPPKPKDTLMISRTPEVTCVVVDVSQEDPEVQ
FNWYVDGVEVHNAKTKPREEQFNSTYRVVSVLTVLHQDWLNGKEYKCKVSNKGLPSS
IEKTISKAKGQPREPQVYTLPPSQEEMTKNQVSLTCLVKGFYPSDIAVEWESNGQPENNY
KTTPPVLDSDGSFFLYSRLTVDKSRWQEGNVFSCSVMHEALHNHYTQKSLSLSLGKAS
Anti-DCIR_29G10.3D9_K-Var1-V-hIgGK-C (SEQ ID NO: 30):
ATGGATTTTCAAGTGCAGATTTTCAGCTTCCTGCTAATGAGTGCCTCAGTCATAATGT
CCAGGGGACAAATTGTTCTCACCCAGTCTCCAGCACTCATGTCTGCATCTCCAGGGG
AGAAGGTCACCATGACCTGCAGTGCCAGCTCAAATATAAGTTACATGTACTGGTACC
AGCAGAAGCCAAGATCCTCCCCCAAACCCTGGATTTATCTCACATCCAACCTGGCTT
CTGGAGTCCCTGCTCGCTTCAGTGGCAGTGGGTCTGGGACCTCTTACTCTCTCACAA
CCAGCAGCATGGAGGCTGAAGATGCTGCCACTTATTGCTGCCAGCAGTGGAGTAGT
AACCCACCCACGTTCGGTGCTGGGACCAAGCTCGAGATCAAACGAACTGTGGCTGC
ACCATCTGTCTTCATCTTCCCGCCATCTGATGAGCAGTTGAAATCTGGAACTGCCTCT
GTTGTGTGCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGACAGCAA
GGACAGCACCTACAGCCTCAGCAGCACCCTGACGCTGAGCAAAGCAGACTACGAGA
AACACAAAGTCTATGCCTGCGAAGTCACCCATCAGGGCCTGAGCTCGCCCGTCACA
AAGAGCTTCAACAGGGGAGAGTGTTAG
Anti-DCIR_29G10.3D9_K-Var1-V-hIgGK-C (SEQ ID NO: 95):
MDFQVQIFSFLLMSASVIMSRGQIVLTQSPALMSASPGEKVTMTCSASSNISYMYWYQQ
KPRSSPKPWIYLTSNLASGVPARFSGSGSGTSYSLTTSSMEAEDAATYCCQQWSSNPPTF
GAGTKLEIKRTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSFNRGEC
Anti-DCIR_29G10.3D9_K-Var2-V-hIgGK-C (SEQ ID NO: 31):
ATGGATTTTCGAGTGCAGATTTTCAGCTTCCTGCTAATGAGTGCCTCAGTCAT
AATGTCCAGGGGACAAATTGTTCTCACCCAGTCTCCAGCACTCATGTCTGCATCTCC
AGGGGAGAAGGTCACCATGACCTGCAGTGCCAGCTCAAATATAAGTTACATGTACT
GGTACCAGCAGAAGCCAAGATCCTCCCCCAAACCCTGGATTTATCTCACATCCAACC
TGGCTTCTGGAGTCCCTGCTCGCTTCAGTGGCAGTGGGTCTGGGACCTCTTACTCTCT
CACAATCAGCAGCATGGAGGCTGAAGATGCTGCCACTTATTACTGCCAGCAGTGGA
GTAGTAACCCACCCACGTTCGGTGCTGGGACCAAGCTCGAGATCAAACGAACTGTG
GCTGCACCATCTGTCTTCATCTTCCCGCCATCTGATGAGCAGTTGAAATCTGGAACT
GCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGA
CAGCAAGGACAGCACCTACAGCCTCAGCAGCACCCTGACGCTGAGCAAAGCAGACT
ACGAGAAACACAAAGTCTATGCCTGCGAAGTCACCCATCAGGGCCTGAGCTCGCCC
GTCACAAAGAGCTTCAACAGGGGAGAGTGTTAG
Anti-DCIR_29G10.3D9_K-Var2-V-hIgGK-C (SEQ ID NO: 96):
MDFRVQIFSFLLMSASVIMSRGQIVLTQSPALMSASPGEKVTMTCSASSNISYMYWYQQ
KPRSSPKPWIYLTSNLASGVPARFSGSGSGTSYSLTISSMEAEDAATYYCQQWSSNPPTF
GAGTKLEIKRTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSFNRGEC
Anti-DCIR_2C9K-V-hIgGK-C (SEQ ID NO: 32):
ATGGAGACAGACACACTCCTGCTATGGGTGCTGCTGCTCTGGGTTCCAGGTTCCACA
GGTGACATTGTGCTGATCCAATCTCCAGCTTCTTTGGCTGTGTCTCTAGGGCAGAGG
GCCACCATATCCTGCAGAGCCAGTGAAAGTGTTGATAGTTATGTCAATAGTTTTATG
CACTGGTACCAGCAGAAACCAGGACAGCCACCCAAACTCCTCATCTATCGTGTATCC
AACCTAGAATCTGGGATCCCTGCCAGGTTCAGTGGCAGTGGGTCTAGGACAGACTTC
ACCCTCACCATTAATCCTGTGGAGGCTGATGATGTTGCAACCTATTACTGTCAGCAA
AGTAATGAGGATCCATTCACGTTCGGCTCGGGGACAAAGCTCGAGATCAAACGAAC
TGTGGCTGCACCATCTGTCTTCATCTTCCCGCCATCTGATGAGCAGTTGAAATCTGGA
ACTGCCTCTGTTGTGTGCCTGCTGAATAACTTCTATCCCAGAGAGGCCAAAGTACAG
TGGAAGGTGGATAACGCCCTCCAATCGGGTAACTCCCAGGAGAGTGTCACAGAGCA
GGACAGCAAGGACAGCACCTACAGCCTCAGCAGCACCCTGACGCTGAGCAAAGCAG
ACTACGAGAAACACAAAGTCTATGCCTGCGAAGTCACCCATCAGGGCCTGAGCTCG
CCCGTCACAAAGAGCTTCAACAGGGGAGAGTGTTAG
Anti-DCIR_2C9K-V-hIgGK-C (SEQ ID NO: 97):
METDTLLLWVLLLWVPGSTGDIVLIQSPASLAVSLGQRATISCRASESVDSYVNS
FMHWYQQKPGQPPKLLIYRVSNLESGIPARFSGSGSRTDFTLTINPVEADDVATYYCQQS
NEDPFTFGSGTKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSPVTKSFN
RGEC.
Anti-DCIR_31A6.1F5_H-var2-V-hIgG4H-C (SEQ ID NO: 33):
ATGGAATGTAACTGGATACTTCCTTTTATTCTGTCGGTAATTTCAGGGGTCTACTCAG
AGGTTCAGCTCCAGCAGTCTGGGACTGTGCTGGCAAGGCCTGGGGCTTCCGTGAATA
TGTCCTGTAAGGCTGCTGGCTACAGCTTTACCAGTTACTGGGTGTACTGGGTCAAAC
AGAGGCCTGGACAGGGTCTGGAATGGATTGGTGCTATTTACCCTAAAAATAGTAGA
ACTAGCTACAACCAGAAGTTCCAGGACAAGGCCACACTGACTGCAGTCACATCCGC
CAGCACTGCCTACATGGAGCTCAGCAGCCTGACAAATGAGGACTCTGCGGTCTATTA
CTGTACAAGACCTCACTATGATTCGTTTGGTTACTGGGGCCAAGGGACTCTGGTCAC
TGTCTCTGCAGCCAAAACAaagggcccATCCGTCTTCCCCCTGGCGCCCTGCTCCAGGAG
CACCTCCGAGAGCACAGCCGCCCTGGGCTGCCTGGTCAAGGACTACTTCCCCGAACC
GGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCA
GCAGCTTGGGCACGAAGACCTACACCTGCAACGTAGATCACAAGCCCAGCAACACC
AAGGTGGACAAGAGAGTTGAGTCCAAATATGGTCCCCCATGCCCACCCTGCCCAGC
ACCTGAGTTCGAAGGGGGACCATCAGTCTTCCTGTTCCCCCCAAAACCCAAGGACAC
TCTCATGATCTCCCGGACCCCTGAGGTCACGTGCGTGGTGGTGGACGTGAGCCAGGA
AGACCCCGAGGTCCAGTTCAACTGGTACGTGGATGGCGTGGAGGTGCATAATGCCA
AGACAAAGCCGCGGGAGGAGCAGTTCAACAGCACGTACCGTGTGGTCAGCGTCCTC
ACCGTCCTGCACCAGGACTGGCTGAACGGCAAGGAGTACAAGTGCAAGGTCTCCAA
CAAAGGCCTCCCGTCCTCCATCGAGAAAACCATCTCCAAAGCCAAAGGGCAGCCCC
GAGAGCCACAGGTGTACACCCTGCCCCCATCCCAGGAGGAGATGACCAAGAACCAG
GTCAGCCTGACCTGCCTGGTCAAAGGCTTCTACCCCAGCGACATCGCCGTGGAGTGG
GAGAGCAATGGGCAGCCGGAGAACAACTACAAGACCACGCCTCCCGTGCTGGACTC
CGACGGCTCCTTCTTCCTCTACAGCAGGCTAACCGTGGACAAGAGCAGGTGGCAGG
AGGGGAATGTCTTCTCATGCTCCGTGATGCATGAGGCTCTGCACAACCACTACACAC
AGAAGAGCCTCTCCCTGTCTCTGGGTAAAGCTAGCTGA
Anti-DCIR_31A6.1F5_H-var2-V-hIgG4H-C (SEQ ID NO: 98):
MECNWILPFILSVISGVYSEVQLQQSGTVLARPGASVNMSCKAAGYSFTSYWVYWVKQ
RPGQGLEWIGAIYPKNSRTSYNQKFQDKATLTAVTSASTAYMELSSLTNEDSAVYYCTR
PHYDSFGYWGQGTLVTVSAAKTKGPSVFPLAPCSRSTSESTAALGCLVKDYFPEPVTVS
WNSGALTSGVHTFPAVLQSSGLYSLSSVVTVPSSSLGTKTYTCNVDHKPSNTKVDKRVE
SKYGPPCPPCPAPEFEGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIEKTISK
AKGQPREPQVYTLPPSQEEMTKNQVSLTCLVKGFYPSDIAVEWESNGQPENNYKTTPPV
LDSDGSFFLYSRLTVDKSRWQEGNVFSCSVMHEALHNHYTQKSLSLSLGKAS
Anti-DCIR_31A6.1F5_K-var2-V-hIgGK-C (SEQ ID NO: 34):
ATGGAGACAGACACACTCCTGCTATGGGTGCTGCTGCTCTGGGTTCCAGGTTCCACA
GGTGACATTGTGCTGACCCAATCTCCAGCTTCTTTGGCTGTGTCTCTAGGGCAGAGG
GCCACCATATCCTGCAGAGCCAGTGAAAGTGTAGATAGTTATGGCATTAGTTTTATG
CACTGGTACCAGCAGAAACCAGGACAGCCACCCAAACTCCTCATCTATCGTGCATCC
AACCAAGAATCTGGGATCCCTGCCAGGTTCAGTGGCAGTGGGTCTAGGACAGACTT
CACCCTCACCATTAATCCTGTGGAGGCTGATGATGTTGCAACCTATTACTGTCAGCA
AAGTAATGAGGATCCGCTCACGTTCGGTGCTGGGACCAAGCTCGAGATCAAACGAA
CTGTGGCTGCACCATCTGTCTTCATCTTCCCGCCATCTGATGAGCAGTTGAAATCTGG
AACTGCCTCTGTTGTGTGCCTGCTGAATAACTTCTATCCCAGAGAGGCCAAAGTACA
GTGGAAGGTGGATAACGCCCTCCAATCGGGTAACTCCCAGGAGAGTGTCACAGAGC
AGGACAGCAAGGACAGCACCTACAGCCTCAGCAGCACCCTGACGCTGAGCAAAGCA
GACTACGAGAAACACAAAGTCTATGCCTGCGAAGTCACCCATCAGGGCCTGAGCTC
GCCCGTCACAAAGAGCTTCAACAGGGGAGAGTGTTAG
Anti-DCIR_31A6.1F5_K-var2-V-hIgGK-C (SEQ ID NO: 99):
METDTLLLWVLLLWVPGSTGDIVLTQSPASLAVSLGQRATISCRASESVDSYGISFMHW
YQQKPGQPPKLLIYRASNQESGIPARFSGSGSRTDFTLTINPVEADDVATYYCQQSNEDP
LTFGAGTKLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
Anti-DCIR_3C2.2D9_H-LV-hIgG4H-C (SEQ ID NO: 35):
ATGAACAGGCTTACTTCCTCATTGCTGCTGCTGATTGTCCCTGCATATGTCCTGTCCC
AGGTTACTCTGAAAGAGTCTGGCCCTGGGATATTGCAGCCCTCCCAGACCCTCAGTC
TGACTTGTTCTTTCTCTGGGTTTTCACTGAGCACTTCTGGTATGGGTGTGAGCTGGAT
TCGTCAGCCTTCAGGAAAGGGTCTGGAGTGGCTGGCACACATTTACTGGGATGATGA
CAAGCGCTATAATCCATCCCTGAAGAGCCGGCTCACAATCTTTAAGGATCCCTCCAG
CAACCAGGTATTCCTCAGGATCACCAGTGTGGACACTGCAGATACTGCCACATACTA
CTGTGCTCGAAACTCCCATTACTACGGTAGTACTTACGGGGGATACTTCGATGTCTG
GGGCGCAGGGACCACGGTCACCGTCTCCTCAGCCAAAACAAAGGGCCCATCCGTCT
TCCCCCTGGCGCCCTGCTCCAGGAGCACCTCCGAGAGCACAGCCGCCCTGGGCTGCC
TGGTCAAGGACTACTTCCCCGAACCGGTGACGGTGTCGTGGAACTCAGGCGCCCTG
ACCAGCGGCGTGCACACCTTCCCGGCTGTCCTACAGTCCTCAGGACTCTACTCCCTC
AGCAGCGTGGTGACCGTGCCCTCCAGCAGCTTGGGCACGAAGACCTACACCTGCAA
CGTAGATCACAAGCCCAGCAACACCAAGGTGGACAAGAGAGTTGAGTCCAAATATG
GTCCCCCATGCCCACCCTGCCCAGCACCTGAGTTCGAAGGGGGACCATCAGTCTTCC
TGTTCCCCCCAAAACCCAAGGACACTCTCATGATCTCCCGGACCCCTGAGGTCACGT
GCGTGGTGGTGGACGTGAGCCAGGAAGACCCCGAGGTCCAGTTCAACTGGTACGTG
GATGGCGTGGAGGTGCATAATGCCAAGACAAAGCCGCGGGAGGAGCAGTTCAACA
GCACGTACCGTGTGGTCAGCGTCCTCACCGTCCTGCACCAGGACTGGCTGAACGGCA
AGGAGTACAAGTGCAAGGTCTCCAACAAAGGCCTCCCGTCCTCCATCGAGAAAACC
ATCTCCAAAGCCAAAGGGCAGCCCCGAGAGCCACAGGTGTACACCCTGCCCCCATC
CCAGGAGGAGATGACCAAGAACCAGGTCAGCCTGACCTGCCTGGTCAAAGGCTTCT
ACCCCAGCGACATCGCCGTGGAGTGGGAGAGCAATGGGCAGCCGGAGAACAACTA
CAAGACCACGCCTCCCGTGCTGGACTCCGACGGCTCCTTCTTCCTCTACAGCAGGCT
AACCGTGGACAAGAGCAGGTGGCAGGAGGGGAATGTCTTCTCATGCTCCGTGATGC
ATGAGGCTCTGCACAACCACTACACACAGAAGAGCCTCTCCCTGTCTCTGGGTAAAG
CTAGCTGA
Anti-DCIR_3C2.2D9_H-LV-hIgG4H-C (SEQ ID NO: 100):
NRLTSSLLLLIVPAYVLSQQVTLKESGPGILQPSQTLSLTCSFSGFSLSTSGMGVSWIRQPS
GKGLEWLAHIYWDDDKRYNPSLKSRLTIFKDPSSNQVFLRITSVDTADTATYYCARNSH
YYGSTYGGYFDVWGAGTTVTVSSAKTKGPSVFPLAPCSRSTSESTAALGCLVKDYFPEP
VTVSWNSGALTSGVHTFPAVLQSSGLYSLSSVVTVPSSSLGTKTYTCNVDHKPSNTKVD
KRVESKYGPPCPPCPAPEFEGGPSVFLFPPKPKDTLMISRTPEVTCVVVDVSQEDPEVQF
NWYVDGVEVHNAKTKPREEQFNSTYRVVSVLTVLHQDWLNGKEYKCKVSNKGLPSSI
EKTISKAKGQPREPQVYTLPPSQEEMTKNQVSLTCLVKGFYPSDIAVEWESNGQPENNY
KTTPPVLDSDGSFFLYSRLTVDKSRWQEGNVFSCSVMHEALHNHYTQKSLSLSLGKAS.
Anti-DCIR_3C2.2D9_K-LV-hIgGK-C (SEQ ID NO: 36):
ATGGAGACAGACACACTCCTGCTATGGGTGCTGCTGCTCGGGGTTCCAGGTTCCACA
GGTAACATTGTGCTGACCCAGTCTCCAACTTCTTTCACTGTGTCTCTTGGGCAGAGG
GCCACCATATCCTGCAGAGCCAGTGAAAGTGTTCATAGTTATGGCAATAGTTTTATG
CACTGGTACCAGCAGAAACCAGGGCAGCCACCCAAACTCCTCATCTATCTTGCATCC
AACGTAGAATCTGGGGTCCCTGCCAGGTTCAGTGGTAGTGGGTCCAGGACAGACTTC
ACCCTCACCATTGATCCTGTGGAGGCTGATGATGCTGCAACCTATTACTGTCAGCAA
AATAGTGAGGATCCGTGGACGTTCGGTGGAGGCACCAAGCTCGAGATCAAACGAAC
TGTGGCTGCACCATCTGTCTTCATCTTCCCGCCATCTGATGAGCAGTTGAAATCTGGA
ACTGCCTCTGTTGTGTGCCTGCTGAATAACTTCTATCCCAGAGAGGCCAAAGTACAG
TGGAAGGTGGATAACGCCCTCCAATCGGGTAACTCCCAGGAGAGTGTCACAGAGCA
GGACAGCAAGGACAGCACCTACAGCCTCAGCAGCACCCTGACGCTGAGCAAAGCAG
ACTACGAGAAACACAAAGTCTATGCCTGCGAAGTCACCCATCAGGGCCTGAGCTCG
CCCGTCACAAAGAGCTTCAACAGGGGAGAGTGTTAG
Anti-DCIR_3C2.2D9_K-LV-hIgGK-C (SEQ ID NO: 101):
METDTLLLWVLLLGVPGSTGNIVLTQSPTSFTVSLGQRATISCRASESVHSYGNSFMHW
YQQKPGQPPKLLIYLASNVESGVPARFSGSGSRTDFTLTIDPVEADDAATYYCQQNSEDP
WTFGGGTKLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
Anti-DCIR_6C8.1G9_H-V-hIgG4H-C (SEQ ID NO: 37):
ATGGAATGGACCTGGGTCTTTCTCTTCCTCCTGTCAGTAACTGCAGGTGTCCA
CTCCCAGGTTCAGCTGCAGCAGTCTGGAACTGAGCTGATGAAGCCTGGGGCCTCAGT
GAAGATATCCTGCAAGGCTACTGGCTACACATTCAGTACCTACTGGATAGAGTGGGT
AAAGCAGAGGCCTGGACATGGCCTTGAGTGGATTGGAGAGATTTTACCTGGAAGTG
GTAGGACTAACGACAATGAGAAGTTCAAGGGCAAGGCCACAATCACTGCAGATACA
TCCTCCAAGAAAGCCTACATGCAACTCAGCAGCCTGACATCTGAGGACTCTGCCGTC
TATTACTGTGCAAGAAGGGGTGGTTACTCCTTTGCTTTCTGGGGCCAAGGGACTCTG
GTCTCTGTCTCTGCAGCCAAAACAAAGGGCCCATCCGTCTTCCCCCTGGCGCCCTGC
TCCAGGAGCACCTCCGAGAGCACAGCCGCCCTGGGCTGCCTGGTCAAGGACTACTT
CCCCGAACCGGTGACGGTGTCGTGGAACTCAGGCGCCCTGACCAGCGGCGTGCACA
CCTTCCCGGCTGTCCTACAGTCCTCAGGACTCTACTCCCTCAGCAGCGTGGTGACCG
TGCCCTCCAGCAGCTTGGGCACGAAGACCTACACCTGCAACGTAGATCACAAGCCC
AGCAACACCAAGGTGGACAAGAGAGTTGAGTCCAAATATGGTCCCCCATGCCCACC
CTGCCCAGCACCTGAGTTCGAAGGGGGACCATCAGTCTTCCTGTTCCCCCCAAAACC
CAAGGACACTCTCATGATCTCCCGGACCCCTGAGGTCACGTGCGTGGTGGTGGACGT
GAGCCAGGAAGACCCCGAGGTCCAGTTCAACTGGTACGTGGATGGCGTGGAGGTGC
ATAATGCCAAGACAAAGCCGCGGGAGGAGCAGTTCAACAGCACGTACCGTGTGGTC
AGCGTCCTCACCGTCCTGCACCAGGACTGGCTGAACGGCAAGGAGTACAAGTGCAA
GGTCTCCAACAAAGGCCTCCCGTCCTCCATCGAGAAAACCATCTCCAAAGCCAAAG
GGCAGCCCCGAGAGCCACAGGTGTACACCCTGCCCCCATCCCAGGAGGAGATGACC
AAGAACCAGGTCAGCCTGACCTGCCTGGTCAAAGGCTTCTACCCCAGCGACATCGC
CGTGGAGTGGGAGAGCAATGGGCAGCCGGAGAACAACTACAAGACCACGCCTCCC
GTGCTGGACTCCGACGGCTCCTTCTTCCTCTACAGCAGGCTAACCGTGGACAAGAGC
AGGTGGCAGGAGGGGAATGTCTTCTCATGCTCCGTGATGCATGAGGCTCTGCACAAC
CACTACACACAGAAGAGCCTCTCCCTGTCTCTGGGTAAAGCTAGCTGA
Anti-DCIR_6C8.1G9_H-V-hIgG4H-C (SEQ ID NO: 102):
MEWTWVFLFLLSVTAGVHSQVQLQQSGTELMKPGASVKISCKATGYTFSTYWIEWVK
QRPGHGLEWIGEILPGSGRTNDNEKFKGKATITADTSSKKAYMQLSSLTSEDSAVYYCA
RRGGYSFAFWGQGTLVSVSAAKTKGPSVFPLAPCSRSTSESTAALGCLVKDYFPEPVTV
SWNSGALTSGVHTFPAVLQSSGLYSLSSVVTVPSSSLGTKTYTCNVDHKPSNTKVDKRV
ESKYGPPCPPCPAPEFEGGPSVFLFPPKPKDTLMISRTPEVTCVVVDVSQEDPEVQFNWY
VDGVEVHNAKTKPREEQFNSTYRVVSVLTVLHQDWLNGKEYKCKVSNKGLPSSIEKTIS
KAKGQPREPQVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSLGKAS
Anti-DCIR_6C8.1G9_K-V-hIgGK-C (SEQ ID NO: 38):
ATGACCATGTTCTCACTAGCTCTTCTCCTCAGTCTTCTTCTCCTCTGTGTCTCT
GATTCTAGGGCAGAAACAACTGTGACCCAGTCTCCAGCATCCCTGTCCATGGCTATA
GGAGAAAAAGTCACCATCAGATGCGTAACCAGCACTGATATTGATGATGATGTGAA
CTGGTACCAGCAGAAGCCAGGGGAACCTCCTAAGCTCCTTATTTCAGAAGGCAATA
CTCTTCGTGCTGGAGTCCCATCCCGATTCTCCAGCAGTGGCTATGGTACAGATTTTGT
TTTTACAATTGAGAACATGCTCTCAGAAGATGTTGCAGATTACTACTGTTTGCAAAG
TGGTAACTTGCCGTACACGTTCGGAGGGGGGACCAAGCTCGAGATCAAACGAACTG
TGGCTGCACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAAGTACAGT
GGAAGGTGGATAACGCCCTCCAATCGGGTAACTCCCAGGAGAGTGTCACAGAGCAG
GACAGCAAGGACAGCACCTACAGCCTCAGCAGCACCCTGACGCTGAGCAAAGCAGA
CTACGAGAAACACAAAGTCTATGCCTGCGAAGTCACCCATCAGGGCCTGAGCTCGC
CCGTCACAAAGAGCTTCAACAGGGGAGAGTGTTAG
Anti-DCIR_6C8.1G9_K-V-hIgGK-C (SEQ ID NO: 103):
MTMFSLALLLSLLLLCVSDSRAETTVTQSPASLSMAIGEKVTIRCVTSTDIDDDVNWYQ
QKPGEPPKLLISEGNTLRAGVPSRFSSSGYGTDFVFTIENMLSEDVADYYCLQSGNLPYT
FGGGTKLEIKRTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPVTKSFNRGEC
Anti-DCIR_9E8.1E3_H-V-hIgG4H-C (SEQ ID NO: 39):
ATGAACAGGCTTACTTCCTCATTGCTGCTGCTGATTGTCCCTGCATATGTCCTGTCCC
AGGTTACTCTGAAAGAGTCTGGCCCTGGGATATTGCAGCCCTCCCAGACCCTCAGTC
TGACTTGTTCTTTCTCTGGGTTTTCACTGAGCACTTCTGGTATGGGTCTGAGCTGGAT
TCGTCAGCCTTCAGGAAAGGGTCTGGAGTGGCTGGCACACATTTACTGGGATGATGA
CAAGCGCTATAACCCATCCCTGAAGAGCCGGCTCACAATCTCCAAGGATACCTCCA
GCAACCAGGTTTTCCTCAAGATCACCATTGTGGACACTGCAGATGCTGCCACATACT
ACTGTGCTCGAAGCTCCCATTACTACGGTTATGGCTACGGGGGATACTTCGATGTCT
GGGGCGCAGGGACCACGGTCACCGTCTCCTCAGCCAAAACGAAGGGCCCATCCGTC
TTCCCCCTGGCGCCCTGCTCCAGGAGCACCTCCGAGAGCACAGCCGCCCTGGGCTGC
CTGGTCAAGGACTACTTCCCCGAACCGGTGACGGTGTCGTGGAACTCAGGCGCCCTG
ACCAGCGGCGTGCACACCTTCCCGGCTGTCCTACAGTCCTCAGGACTCTACTCCCTC
AGCAGCGTGGTGACCGTGCCCTCCAGCAGCTTGGGCACGAAGACCTACACCTGCAA
CGTAGATCACAAGCCCAGCAACACCAAGGTGGACAAGAGAGTTGAGTCCAAATATG
GTCCCCCATGCCCACCCTGCCCAGCACCTGAGTTCGAAGGGGGACCATCAGTCTTCC
TGTTCCCCCCAAAACCCAAGGACACTCTCATGATCTCCCGGACCCCTGAGGTCACGT
GCGTGGTGGTGGACGTGAGCCAGGAAGACCCCGAGGTCCAGTTCAACTGGTACGTG
GATGGCGTGGAGGTGCATAATGCCAAGACAAAGCCGCGGGAGGAGCAGTTCAACA
GCACGTACCGTGTGGTCAGCGTCCTCACCGTCCTGCACCAGGACTGGCTGAACGGCA
AGGAGTACAAGTGCAAGGTCTCCAACAAAGGCCTCCCGTCCTCCATCGAGAAAACC
ATCTCCAAAGCCAAAGGGCAGCCCCGAGAGCCACAGGTGTACACCCTGCCCCCATC
CCAGGAGGAGATGACCAAGAACCAGGTCAGCCTGACCTGCCTGGTCAAAGGCTTCT
ACCCCAGCGACATCGCCGTGGAGTGGGAGAGCAATGGGCAGCCGGAGAACAACTA
CAAGACCACGCCTCCCGTGCTGGACTCCGACGGCTCCTTCTTCCTCTACAGCAGGCT
AACCGTGGACAAGAGCAGGTGGCAGGAGGGGAATGTCTTCTCATGCTCCGTGATGC
ATGAGGCTCTGCACAACCACTACACACAGAAGAGCCTCTCCCTGTCTCTGGGTAAAG
CTAGCTGA
Anti-DCIR_9E8.1E3_H-V-hIgG4H-C (SEQ ID NO: 104):
MNRLTSSLLLLIVPAYVLSQVTLKESGPGILQPSQTLSLTCSFSGFSLSTSGMGLSWIRQPS
GKGLEWLAHIYWDDDKRYNPSLKSRLTISKDTSSNQVFLKITIVDTADAATYYCARSSH
YYGYGYGGYFDVWGAGTTVTVSSAKTKGPSVFPLAPCSRSTSESTAALGCLVKDYFPEP
VTVSWNSGALTSGVHTFPAVLQSSGLYSLSSVVTVPSSSLGTKTYTCNVDHKPSNTKVD
KRVESKYGPPCPPCPAPEFEGGPSVFLFPPKPKDTLMISRTPEVTCVVVDVSQEDPEVQF
NWYVDGVEVHNAKTKPREEQFNSTYRVVSVLTVLHQDWLNGKEYKCKVSNKGLPSSI
EKTISKAKGQPREPQVYTLPPSQEEMTKNQVSLTCLVKGFYPSDIAVEWESNGQPENNY
KTTPPVLDSDGSFFLYSRLTVDKSRWQEGNVFSCSVMHEALHNHYTQKSLSLSLGKAS
Anti-DCIR_9E8.1E3_K-LV-hIgGK-C (SEQ ID NO: 40):
ATGGAGACAGACACACTCCTGCTATGGGTGCTGCTGCTCTGGGTTCCAGGTTC
CACAGGTAACATTGTGCTGACCCAATCTCCAGCTTCTTTGGCTGTGTCTCTAGGGCA
GAGGGCCACCATATCCTGCAGAGCCAGTGAAAGTATTCATAGTTATGGCAATAGTTT
TCTGCACTGGTACCAGCAGAAACCAGGACAGCCACCCAAACTCCTCATCTATCTTGC
ATCCAACCTAGAATCTGGGGTCCCTGCCAGGTTCAGCGGCAGTGGGTCTAGGACAG
ACTTCACCCTCACCATTGATCCTGTGGAGGCTGATGATGCTGCAACCTATTACTGTC
AGCAAAATAATGAGGATCCGTGGACGTTCGGTGGAGGCACCAAGCTCGAGATCAAA
CGAACTGTGGCTGCACCATCTGTCTTCATCTTCCCGCCATCTGATGAGCAGTTGAAA
TCTGGAACTGCCTCTGTTGTGTGCCTGCTGAATAACTTCTATCCCAGAGAGGCCAAA
GTACAGTGGAAGGTGGATAACGCCCTCCAATCGGGTAACTCCCAGGAGAGTGTCAC
AGAGCAGGACAGCAAGGACAGCACCTACAGCCTCAGCAGCACCCTGACGCTGAGCA
AAGCAGACTACGAGAAACACAAAGTCTATGCCTGCGAAGTCACCCATCAGGGCCTG
AGCTCGCCCGTCACAAAGAGCTTCAACAGGGGAGAGTGTTAGGCGGCCGCACTAGC
GCGGGCCGCATTCGAAGAGCTCGGTACCCGGGGATCCTCTAGAGTCGACCTGCAGG
CATGCAAGCTGGCCGCGACTCTAGATCATAATCAGC
Anti-DCIR_9E8.1E3_K-LV-hIgGK-C (SEQ ID NO: 105):
METDTLLLWVLLLWVPGSTGNIVLTQSPASLAVSLGQRATISCRASESIHSYGNSFLHWY
QQKPGQPPKLLIYLASNLESGVPARFSGSGSRTDFTLTIDPVEADDAATYYCQQNNEDP
WTFGGGTKLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
Anti-DCIR2C9H-LV-hIgG4H-V-hIgG4H-C (SEQ ID NO: 41):
ATGAAATGCAGCTGGGTCATCTTCTTCCTGATGGCAGTGGTTACAGGGGTCAATTCA
GAGGTTCAGCTGCAGCAGTCTGGGGCTGAGCTTGTGAGGCCAGGGGCCTTAGTCAA
GTTGTCCTGCAAAGCTTCTGGCTTCAACATTAATGACTACTATATCCACTGGGTGAA
GCAGCGGCCTGAACAGGGCCTGGAGCGGATTGGATGGATTGATCCTGACAATGGTA
ATACTATATATGACCCGAAGTTCCAGGGCAAGGCCAGTATAACAGCAGACACATCC
CCCAACACAGCCTACCTGCAGCTCAGCAGCCTGACATCTGAGGACACTGCCGTCTAT
TACTGTGCTAGAACCCGATCTCCTATGGTTACGACGGGGTTTGTTTACTGGGGCCAA
GGGACTGTGGTCACTGTCTCTGCAGCCAAAACGAAGGGCCCATCCGTCTTCCCCCTG
GCGCCCTGCTCCAGGAGCACCTCCGAGAGCACAGCCGCCCTGGGCTGCCTGGTCAA
GGACTACTTCCCCGAACCGGTGACGGTGTCGTGGAACTCAGGCGCCCTGACCAGCG
GCGTGCACACCTTCCCGGCTGTCCTACAGTCCTCAGGACTCTACTCCCTCAGCAGCG
TGGTGACCGTGCCCTCCAGCAGCTTGGGCACGAAGACCTACACCTGCAACGTAGAT
CACAAGCCCAGCAACACCAAGGTGGACAAGAGAGTTGAGTCCAAATATGGTCCCCC
ATGCCCACCCTGCCCAGCACCTGAGTTCGAAGGGGGACCATCAGTCTTCCTGTTCCC
CCCAAAACCCAAGGACACTCTCATGATCTCCCGGACCCCTGAGGTCACGTGCGTGGT
GGTGGACGTGAGCCAGGAAGACCCCGAGGTCCAGTTCAACTGGTACGTGGATGGCG
TGGAGGTGCATAATGCCAAGACRAAGCCGCGGGAGGAGCAGTTCAACAGCACGTAC
CGTGTGGTCAGCGTCCTCACCGTCCTGCACCAGGACTGGCTGAACGGCAAGGAGTA
CAAGTGCAAGGTCTCCAACAAAGGCCTCCCGTCCTCCATCGAGAAAACCATCTCCA
AAGCCAAAGGGCAGCCCCGAGAGCCACAGGTGTACACCCTGCCCCCATCCCAGGAG
GAGATGACCAAGAACCAGGTCAGCCTGACCTGCCTGGTCAAAGGCTTCTACCCCAG
CGACATCGCCGTGGAGTGGGAGAGCAATGGGCAGCCGGAGAACAACTACAAGACC
ACGCCTCCCGTGCTGGACTCCGACGGCTCCTTCTTCCTCTACAGCAGGCTAACCGTG
GACAAGAGCAGGTGGCAGGAGGGGAATGTCTTCTCATGCTCCGTGATGCATGAGGC
TCTGCACAACCACTACACACAGAAGAGCCTCTCCCTGTCTCTGGGTAAATGA
Anti-DCIR2C9H-LV-hIgG4H-V-hIgG4H-C (SEQ ID NO: 106):
MKCSWVIFFLMAVVTGVNSEVQLQQSGAELVRPGALVKLSCKASGFNINDYYIHWVKQ
RPEQGLERIGWIDPDNGNTIYDPKFQGKASITADTSPNTAYLQLSSLTSEDTAVYYCART
RSPMVTTGFVYWGQGTVVTVSAAKTKGPSVFPLAPCSRSTSESTAALGCLVKDYFPEPV
TVSWNSGALTSGVHTFPAVLQSSGLYSLSSVVTVPSSSLGTKTYTCNVDHKPSNTKVDK
RVESKYGPPCPPCPAPEFEGGPSVFLFPPKPKDTLMISRTPEVTCVVVDVSQEDPEVQFN
WYVDGVEVHNAKXKPREEQFNSTYRVVSVLTVLHQDWLNGKEYKCKVSNKGLPSSIE
KTISKAKGQPREPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSLSLSLGK
Anti-DC-SIGNL16E3H (SEQ ID NO: 42):
ATGGAAAGGCACTGGATCTTTCTCTTCCTGTTTTCAGTAACTGCAGGTGTCCA
CTCCCAGGTCCAGCTTCAGCAGTCTGGGGCTGAGCTGGCAAAACCTGGGGCCTCAGT
GAAGATGTCCTGCAAGGCTTCTGGCTACACCTTTACTACCTACTGGATGCACTGGGT
AAAACAGAGGCCTGGACAGGGTCTGGAATGGATTGGATACATTAATCCTATCACTG
GTTATACTGAGTACAATCAGAAGTTCAAGGACAAGGCCACCTTGACTGCAGACAAA
TCCTCCAGCACAGCCTACATGCAACTGAGCAGCCTGACATCTGAGGACTCTGCAGTC
TATTACTGTGCAAGAGAGGGTTTAAGTGCTATGGACTATTGGGGTCAGGGAACCTCA
GTCACCGTCACCTCAGCCAAAACAACAGCCCCATCGGTCTATCCACTGGCCCCTGTG
TGTGGAGATACAACTGGCTCCTCGGTAACTCTAGGATGCCTGGTCAAGGGTTATTTC
CCTGAGCCAGTGACCTTGACCTGGAACTCTGGATCCCTGTCCAGTGGTGTGCACACC
TTCCCAGCTGTCCTGCAGTCTGACCTCTACACCCTCAGCAGCTCAGTGACTGTAACC
TCGAGCACCTGGCCCAGCCAGACCGTCACCTGCAGCGTTGCTCACCCAGCCAGCAG
CACCACGGTGGACAAAAAACTTGAGCCCAGCGGGCCCATTTCAACAATCAACCCCT
GTCCTCCATGCAAGGAGTGTCACAAATGCCCAGCTCCTAACCTCGAGGGTGGACCAT
CCGTCTTCATCTTCCCTCCAAATATCAAGGATGTACTCATGATCTCCCTGACACCCAA
GGTCACGTGTGTGGTGGTGGATGTGAGCGAGGATGACCCAGACGTCCAGATCAGCT
GGTTTGTGAACAACGTGGAAGTACACACAGCTCAGACACAAACCCATAGAGAGGAT
TACAACAGTACTATCCGGGTGGTCAGCACCCTCCCCATCCAGCACCAGGACTGGATG
AGTGGCAAGGAGTTCAAATGCAAGGTCAACAACAAAGACCTCCCATCACCCATCGA
GAGAACCATCTCAAAAATTAAAGGGCTAGTCAGAGCTCCACAAGTATACATCTTGC
CGCCACCAGCAGAGCAGTTGTCCAGGAAAGATGTCAGTCTCACTTGCCTGGTCGTGG
GCTTCAACCCTGGAGACATCAGTGTGGAGTGGACCAGCAATGGGCATACAGAGGAG
AACTACAAGGACACCGCACCAGTCCTGGACTCTGACGGTTCTTACTTCATATATAGC
AAGCTCAATATGAAAACAAGCAAGTGGGAGAAAACAGATTCCTTCTCATGCAACGT
GAGACACGAGGGTCTGAAAAATTACTACCTGAAGAAGACCATCTCCCGGTCTCCGG
GTAAAGCTAGCTGA
Anti-DC-SIGNL16E3H (SEQ ID NO: 107):
MERHWIFLFLFSVTAGVHSQVQLQQSGAELAKPGASVKMSCKASGYTFTTYWMHWVK
QRPGQGLEWIGYINPITGYTEYNQKFKDKATLTADKSSSTAYMQLSSLTSEDSAVYYCA
REGLSAMDYWGQGTSVTVTSAKTTAPSVYPLAPVCGDTTGSSVTLGCLVKGYFPEPVT
LTWNSGSLSSGVHTFPAVLQSDLYTLSSSVTVTSSTWPSQTVTCSVAHPASSTTVDKKLE
PSGPISTINPCPPCKECHKCPAPNLEGGPSVFIFPPNIKDVLMISLTPKVTCVVVDVSEDDP
DVQISWFVNNVEVHTAQTQTHREDYNSTIRVVSTLPIQHQDWMSGKEFKCKVNNKDLP
SPIERTISKIKGLVRAPQVYILPPPAEQLSRKDVSLTCLVVGFNPGDISVEWTSNGHTEEN
YKDTAPVLDSDGSYFIYSKLNMKTSKWEKTDSFSCNVRHEGLKNYYLKKTISRSPGKAS
Anti-DC-SIGNL16E3K (SEQ ID NO: 43):
ATGGGCATCAAGATGGAGTCACGGATTCAGGCATTTGTATTCGTGTTTCTCTG
GTTGTCTGGTGTTGGCGGAGACATTGTGATGACCCAGTCTCACAAATTCATGTCCAC
ATCAGTAGGAGACAGGGTCAGCGTCACCTGCAAGGCCAGTCAGGATGTGACTTCTG
CTGTAGCCTGGTATCAACAAAAACCAGGGCAATCTCCTAAACTACTGATTTACTGGG
CATCCACCCGGCACACTGGAGTCCCTGATCGCTTCACAGGCAGTGGATCTGGGACA
GATTATACTCTCACCATCAGCAGTGGGCAGGCTGAAGACCTGGCACTTTATTACTGT
CACCAATATTATAGCGCTCCTCGGACGTTCGGTGGAGGCACCAAGCTGGAAGTCAA
ACGGGCTGATGCTGCACCAACTGTATCCATCTTCCCACCATCCAGTGAGCAGTTAAC
ATCTGGAGGTGCCTCAGTCGTGTGCTTCTTGAACAACTTCTACCCCAAAGACATCAA
TGTCAAGTGGAAGATTGATGGCAGTGAACGACAAAATGGCGTCCTGAACAGTTGGA
CTGATCAGGACAGCAAAGACAGCACCTACAGCATGAGCAGCACCCTCACGTTGACC
AAGGACGAGTATGAACGACATAACAGCTATACCTGTGAGGCCACTCACAAGACATC
AACTTCACCCATCGTCAAGAGCTTCAATAGGAATGAGTGTTAG
Anti-DC-SIGNL16E3K (SEQ ID NO: 108):
MESRIQAFVFVFLWLSGVGGDIVMTQSHKFMSTSVGDRVSVTCKASQDVTSAVAWYQ
QKPGQSPKLLIYWASTRHTGVPDRFTGSGSGTDYTLTISSGQAEDLALYYCHQYYSAPR
TFGGGTKLEVKRADAAPTVSIFPPSSEQLTSGGASVVCFLNNFYPKDINVKWKIDGSERQ
NGVLNSWTDQDSKDSTYSMSSTLTLTKDEYERHNSYTCEATHKTSTSPIVKSFNRNEC
Anti-DC-SIGNL16E7H-LV-hIgG4H-C (SEQ ID NO: 44):
ATGGAAAGGCACTGGATCTTTCTCTTCCTGTTTTCAGTAACTGCAGGTGTCCA
CTCCCAGGTCCAGCTTCAGCAGTCTGGGGCTGAGCTGGCAAAACCTGGGGCCTCAGT
GAAGATGTCCTGCAAGGCTTCTGGCTACACCTTTACTACCTACTGGATGCACTGGGT
AAAACAGAGGCCTGGACAGGGTCTGGAATGGATTGGATACATTAATCCTATCACTG
GTTATACTGAGTACAATCAGAAGTTCAAGGACAAGGCCACCTTGACTGCAGACAAA
TCCTCCAGCACAGCCTACATGCAACTGAGCAGCCTGACATCTGAGGACTCTGCAGTC
TATTACTGTGCAAGAGAGGGTTTAAGTGCTATGGACTATTGGGGTCAGGGAACCTCA
GTCACCGTCACCTCAGCCAAAACAACGGGCCCATCCGTCTTCCCCCTGGCGCCCTGC
TCCAGGAGCACCTCCGAGAGCACAGCCGCCCTGGGCTGCCTGGTCAAGGACTACTT
CCCCGAACCGGTGACGGTGTCGTGGAACTCAGGCGCCCTGACCAGCGGCGTGCACA
CCTTCCCGGCTGTCCTACAGTCCTCAGGACTCTACTCCCTCAGCAGCGTGGTGACCG
TGCCCTCCAGCAGCTTGGGCACGAAGACCTACACCTGCAACGTAGATCACAAGCCC
AGCAACACCAAGGTGGACAAGAGAGTTGAGTCCAAATATGGTCCCCCATGCCCACC
CTGCCCAGCACCTGAGTTCGAAGGGGGACCATCAGTCTTCCTGTTCCCCCCAAAACC
CAAGGACACTCTCATGATCTCCCGGACCCCTGAGGTCACGTGCGTGGTGGTGGACGT
GAGCCAGGAAGACCCCGAGGTCCAGTTCAACTGGTACGTGGATGGCGTGGAGGTGC
ATAATGCCAAGACAAAGCCGCGGGAGGAGCAGTTCAACAGCACGTACCGTGTGGTC
AGCGTCCTCACCGTCCTGCACCAGGACTGGCTGAACGGCAAGGAGTACAAGTGCAA
GGTCTCCAACAAAGGCCTCCCGTCCTCCATCGAGAAAACCATCTCCAAAGCCAAAG
GGCAGCCCCGAGAGCCACAGGTGTACACCCTGCCCCCATCCCAGGAGGAGATGACC
AAGAACCAGGTCAGCCTGACCTGCCTGGTCAAAGGCTTCTACCCCAGCGACATCGC
CGTGGAGTGGGAGAGCAATGGGCAGCCGGAGAACAACTACAAGACCACGCCTCCC
GTGCTGGACTCCGACGGCTCCTTCTTCCTCTACAGCAGGCTAACCGTGGACAAGAGC
AGGTGGCAGGAGGGGAATGTCTTCTCATGCTCCGTGATGCATGAGGCTCTGCACAAC
CACTACACACAGAAGAGCCTCTCCCTGTCTCTGGGTAAAGCTAGCTGA
Anti-DC-SIGNL16E7H-LV-hIgG4H-C (SEQ ID NO: 109):
MERHWIFLFLFSVTAGVHSQVQLQQSGAELAKPGASVKMSCKASGYTFTTYWMHWVK
QRPGQGLEWIGYINPITGYTEYNQKFKDKATLTADKSSSTAYMQLSSLTSEDSAVYYCA
REGLSAMDYWGQGTSVTVTSAKTTGPSVFPLAPCSRSTSESTAALGCLVKDYFPEPVTV
SWNSGALTSGVHTFPAVLQSSGLYSLSSVVTVPSSSLGTKTYTCNVDHKPSNTKVDKRV
ESKYGPPCPPCPAPEFEGGPSVFLFPPKPKDTLMISRTPEVTCVVVDVSQEDPEVQFNWY
VDGVEVHNAKTKPREEQFNSTYRVVSVLTVLHQDWLNGKEYKCKVSNKGLPSSIEKTIS
KAKGQPREPQVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSLGKAS.
Anti-DC-SIGNL16E7K-LV-hIgGK-C (SEQ ID NO: 45):
ATGGGCATCAAGATGGAGTCACAGATTCAGGCATTTGTATTCGTGTTTCTCTG
GTTGTCTGGTGTTGGCGGAGACATTGTGATGACCCAGTCTCACAAATTCATGTCCAC
ATCAGTAGGAGACAGGGTCAGCGTCACCTGCAAGGCCAGTCAGGATGTGACTTCTG
CTGTAGCCTGGTATCAACAAAAACCAGGGCAATCTCCTAAACTACTGATTTACTGGG
CATCCACCCGGCACACTGGAGTCCCTGATCGCTTCACAGGCAGTGGATCTGGGACA
GATTATACTCTCACCATCAGCAGTGGGCAGGCTGAAGACCTGGCACTTTATTACTGT
CACCAATATTATAGCGCTCCTCGGACGTTCGGTGGAGGCACCAAGCTCGAGATCAA
ACGAACTGTGGCTGCACCATCTGTCTTCATCTTCCCGCCATCTGATGAGCAGTTGAA
ATCTGGAACTGCCTCTGTTGTGTGCCTGCTGAATAACTTCTATCCCAGAGAGGCCAA
AGTACAGTGGAAGGTGGATAACGCCCTCCAATCGGGTAACTCCCAGGAGAGTGTCA
CAGAGCAGGACAGCAAGGACAGCACCTACAGCCTCAGCAGCACCCTGACGCTGAGC
AAAGCAGACTACGAGAAACACAAAGTCTATGCCTGCGAAGTCACCCATCAGGGCCT
GAGCTCGCCCGTCACAAAGAGCTTCAACAGGGGAGAGTGTTAG
Anti-DC-SIGNL16E7K-LV-hIgGK-C (SEQ ID NO: 110):
MESQIQAFVFVFLWLSGVGGDIVMTQSHKFMSTSVGDRVSVTCKASQDVTSAVAWYQ
QKPGQSPKLLIYWASTRHTGVPDRFTGSGSGTDYTLTISSGQAEDLALYYCHQYYSAPR
TFGGGTKLEIKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTKSFNRGEC
Anti-Dectin_1_11B6.4_H-V-hIgG4H-C (SEQ ID NO: 46):
ATGGCTGTCCTGGCACTACTCCTCTGCCTGGTGGCTTTCCCAACTTGTACCCT
GTCCCAGGTGCAACTGAAGGAGTCAGGACCTGGCCTGGTGGCGCCCTCACAGAGCC
TGTCCATTACCTGCTCTGTCTCTGGGTTCTCATTAAGCAACTATGATATAAGCTGGAT
TCGCCAGCCACCAGGAAAGGGTCTGGAGTGGCTTGGAGTAATGTGGACTGGTGGAG
GCGCAAATTATAATTCAGCTTTCATGTCCAGACTGAGCATCAACAAGGACAACTCCA
AGAGCCAAGTTTTTTTAAAAATGAACAATCTGCAAACTGATGACACAGCCATTTATT
ACTGTGTCAGAGATGCGGTGAGGTACTGGAACTTCGATGTCTGGGGCGCAGGGACC
ACGGTCACCGTCTCCTCAGCCAAAACGAAGGGCCCATCCGTCTTCCCCCTGGCGCCC
TGCTCCAGGAGCACCTCCGAGAGCACAGCCGCCCTGGGCTGCCTGGTCAAGGACTA
CTTCCCCGAACCGGTGACGGTGTCGTGGAACTCAGGCGCCCTGACCAGCGGCGTGC
ACACCTTCCCGGCTGTCCTACAGTCCTCAGGACTCTACTCCCTCAGCAGCGTGGTGA
CCGTGCCCTCCAGCAGCTTGGGCACGAAGACCTACACCTGCAACGTAGATCACAAG
CCCAGCAACACCAAGGTGGACAAGAGAGTTGAGTCCAAATATGGTCCCCCATGCCC
ACCCTGCCCAGCACCTGAGTTCGAAGGGGGACCATCAGTCTTCCTGTTCCCCCCAAA
ACCCAAGGACACTCTCATGATCTCCCGGACCCCTGAGGTCACGTGCGTGGTGGTGGA
CGTGAGCCAGGAAGACCCCGAGGTCCAGTTCAACTGGTACGTGGATGGCGTGGAGG
TGCATAATGCCAAGACAAAGCCGCGGGAGGAGCAGTTCAACAGCACGTACCGTGTG
GTCAGCGTCCTCACCGTCCTGCACCAGGACTGGCTGAACGGCAAGGAGTACAAGTG
CAAGGTCTCCAACAAAGGCCTCCCGTCCTCCATCGAGAAAACCATCTCCAAAGCCA
AAGGGCAGCCCCGAGAGCCACAGGTGTACACCCTGCCCCCATCCCAGGAGGAGATG
ACCAAGAACCAGGTCAGCCTGACCTGCCTGGTCAAAGGCTTCTACCCCAGCGACAT
CGCCGTGGAGTGGGAGAGCAATGGGCAGCCGGAGAACAACTACAAGACCACGCCT
CCCGTGCTGGACTCCGACGGCTCCTTCTTCCTCTACAGCAGGCTAACCGTGGACAAG
AGCAGGTGGCAGGAGGGGAATGTCTTCTCATGCTCCGTGATGCATGAGGCTCTGCAC
AACCACTACACACAGAAGAGCCTCTCCCTGTCTCTGGGTAAAGCTAGCTGA
Anti-Dectin_1_11B6.4_H-V-hIgG4H-C (SEQ ID NO: 111):
MAVLALLLCLVAFPTCTLSQVQLKESGPGLVAPSQSLSITCSVSGFSLSNYDISWIRQPPG
KGLEWLGVMWTGGGANYNSAFMSRLSINKDNSKSQVFLKMNNLQTDDTAIYYCVRDA
VRYWNFDVWGAGTTVTVSSAKTKGPSVFPLAPCSRSTSESTAALGCLVKDYFPEPVTVS
WNSGALTSGVHTFPAVLQSSGLYSLSSVVTVPSSSLGTKTYTCNVDHKPSNTKVDKRVE
SKYGPPCPPCPAPEFEGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIEKTISK
AKGQPREPQVYTLPPSQEEMTKNQVSLTCLVKGFYPSDIAVEWESNGQPENNYKTTPPV
LDSDGSFFLYSRLTVDKSRWQEGNVFSCSVMHEALHNHYTQKSLSLSLGKAS
Anti-Dectin_1_11B6.4_K-LV-hIgGK-C (SEQ ID NO: 47):
ATGGATTTTCAAGCGCAGATTTTCAGCTTCCTGCTAATCAGTGCTTCAGTCAT
AATGTCCAGAGGACAAATTGTTCTCTCCCAGTCACCAGCAATCCTGTCTGCATCTCC
AGGGGAGAAGGTCACAATGACTTGCAGGGCCAGCTCAAGTGTAAGTTACATACACT
GGTACCAGCAGAAGCCAGGATCCTCCCCCAAACCCTGGATTTATGCCACATCCCACC
TGGCTTCTGGAGTCCCTGCTCGCTTCAGTGGCAGTGGGTCTGGGACCTCTTACTCTCT
CACAATCAGCAGAGTGGAGGCTGAAGATACTGCCACTTATTACTGCCAGCAGTGGA
GTAGTAACCCATTCACGTTCGGCTCGGGGACAAAGCTCGAGATCAAACGAACTGTG
GCTGCACCATCTGTCTTCATCTTCCCGCCATCTGATGAGCAGTTGAAATCTGGAACT
GCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGA
CAGCAAGGACAGCACCTACAGCCTCAGCAGCACCCTGACGCTGAGCAAAGCAGACT
ACGAGAAACACAAAGTCTATGCCTGCGAAGTCACCCATCAGGGCCTGAGCTCGCCC
GTCACAAAGAGCTTCAACAGGGGAGAGTGTTAG
Anti-Dectin_1_11B6.4_K-LV-hIgGK-C (SEQ ID NO: 112):
MDFQAQIFSFLLISASVIMSRGQIVLSQSPAILSASPGEKVTMTCRASSSVSYIHWYQQKP
GSSPKPWIYATSHLASGVPARFSGSGSGTSYSLTISRVEAEDTATYYCQQWSSNPFTFGS
GTKLEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC
Anti-Dectin_1_15E2.5_H-V-hIgG4H-C (SEQ ID NO: 48):
ATGGAAAGGCACTGGATCTTTCTACTCCTGTTGTCAGTAACTGCAGGTGTCCACTCC
CAGGTCCAGCTGCAGCAGTCTGGGGCTGAACTGGCAAGACCTGGGGCCTCAGTGAA
GATGTCCTGCAAGGCTTCTGGCTACACCTTTACTACCTACACTATGCACTGGGTAAA
ACAGAGGCCTGGACAGGGTCTGGAATGGATTGGATACATTAATCCTAGCAGTGGTT
ATACTAATTACAATCAGAAGTTCAAGGACAAGGCCACATTGACTGCAGACAAATCC
TCCAGCACAGCCTCCATGCAACTGAGCAGCCTGACATCTGAGGACTCTGCAGTCTAT
TACTGTGCAAGAGAGAGGGCGGTATTAGTCCCCTATGCTATGGACTACTGGGGTCAA
GGAACCTCAGTCACCGTCTCCTCAGCCAAAACAAAGGGCCCATCCGTCTTCCCCCTG
GCGCCCTGCTCCAGGAGCACCTCCGAGAGCACAGCCGCCCTGGGCTGCCTGGTCAA
GGACTACTTCCCCGAACCGGTGACGGTGTCGTGGAACTCAGGCGCCCTGACCAGCG
GCGTGCACACCTTCCCGGCTGTCCTACAGTCCTCAGGACTCTACTCCCTCAGCAGCG
TGGTGACCGTGCCCTCCAGCAGCTTGGGCACGAAGACCTACACCTGCAACGTAGAT
CACAAGCCCAGCAACACCAAGGTGGACAAGAGAGTTGAGTCCAAATATGGTCCCCC
ATGCCCACCCTGCCCAGCACCTGAGTTCGAAGGGGGACCATCAGTCTTCCTGTTCCC
CCCAAAACCCAAGGACACTCTCATGATCTCCCGGACCCCTGAGGTCACGTGCGTGGT
GGTGGACGTGAGCCAGGAAGACCCCGAGGTCCAGTTCAACTGGTACGTGGATGGCG
TGGAGGTGCATAATGCCAAGACAAAGCCGCGGGAGGAGCAGTTCAACAGCACGTAC
CGTGTGGTCAGCGTCCTCACCGTCCTGCACCAGGACTGGCTGAACGGCAAGGAGTA
CAAGTGCAAGGTCTCCAACAAAGGCCTCCCGTCCTCCATCGAGAAAACCATCTCCA
AAGCCAAAGGGCAGCCCCGAGAGCCACAGGTGTACACCCTGCCCCCATCCCAGGAG
GAGATGACCAAGAACCAGGTCAGCCTGACCTGCCTGGTCAAAGGCTTCTACCCCAG
CGACATCGCCGTGGAGTGGGAGAGCAATGGGCAGCCGGAGAACAACTACAAGACC
ACGCCTCCCGTGCTGGACTCCGACGGCTCCTTCTTCCTCTACAGCAGGCTAACCGTG
GACAAGAGCAGGTGGCAGGAGGGGAATGTCTTCTCATGCTCCGTGATGCATGAGGC
TCTGCACAACCACTACACACAGAAGAGCCTCTCCCTGTCTCTGGGTAAAGCTAGCTGA
Anti-Dectin_1_15E2.5_H-V-hIgG4H-C (SEQ ID NO: 113):
MERHWIFLLLLSVTAGVHSQVQLQQSGAELARPGASVKMSCKASGYTFTTYTMHWVK
QRPGQGLEWIGYINPSSGYTNYNQKFKDKATLTADKSSSTASMQLSSLTSEDSAVYYCA
RERAVLVPYAMDYWGQGTSVTVSSAKTKGPSVFPLAPCSRSTSESTAALGCLVKDYFPE
PVTVSWNSGALTSGVHTFPAVLQSSGLYSLSSVVTVPSSSLGTKTYTCNVDHKPSNTKV
DKRVESKYGPPCPPCPAPEFEGGPSVFLFPPKPKDTLMISRTPEVTCVVVDVSQEDPEVQ
FNWYVDGVEVHNAKTKPREEQFNSTYRVVSVLTVLHQDWLNGKEYKCKVSNKGLPSS
IEKTISKAKGQPREPQVYTLPPSQEEMTKNQVSLTCLVKGFYPSDIAVEWESNGQPENNY
KTTPPVLDSDGSFFLYSRLTVDKSRWQEGNVFSCSVMHEALHNHYTQKSLSLSLGKAS
Anti-Dectin_1_15E2.5_K-V-hIgGK-C (SEQ ID NO: 49):
ATGCATTTTCAAGTGCAGATTTTCAGCTTCCTGCTAATCAGTGCCTCAGTCAT
AATGTCCAGAGGACAAATTGTTCTCACCCAGTCTCCAGCAGTCATGTCTGCATCTCC
AGGGGAGAAGGTCACCATAACCTGCACTGCCAGCTCAAGTTTAAGTTACATGCACT
GGTTCCAGCAGAAGCCAGGCACTTCTCCCAAACTCTGGCTTTATAGCACATCCATCC
TGGCTTCTGGAGTCCCTACTCGCTTCAGTGGCAGTGGATCTGGGACCTCTTACTCTCT
CACAATCAGCCGAATGGAGGCTGAAGATGCTGCCACTTATTACTGCCAGCAAAGGA
GTAGTTCCCCATTCACGTTCGGCTCGGGGACAAAGCTCGAGATCAAACGAACTGTGG
CTGCACCATCTGTCTTCATCTTCCCGCCATCTGATGAGCAGTTGAAATCTGGAACTGC
CTCTGTTGTGTGCCTGCTGAATAACTTCTATCCCAGAGAGGCCAAAGTACAGTGGAA
GGTGGATAACGCCCTCCAATCGGGTAACTCCCAGGAGAGTGTCACAGAGCAGGACA
GCAAGGACAGCACCTACAGCCTCAGCAGCACCCTGACGCTGAGCAAAGCAGACTAC
GAGAAACACAAAGTCTATGCCTGCGAAGTCACCCATCAGGGCCTGAGCTCGCCCGT
CACAAAGAGCTTCAACAGGGGAGAGTGTTAG
Anti-Dectin_1_15E2.5_K-V-hIgGK-C (SEQ ID NO: 114):
MHFQVQIFSFLLISASVIMSRGQIVLTQSPAVMSASPGEKVTITCTASSSLSYMHWFQQKP
GTSPKLWLYSTSILASGVPTRFSGSGSGTSYSLTISRMEAEDAATYYCQQRSSSPFTFGSG
TKLEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
Anti-Dectin_1_2D8.2D4H-V-hIgG4H-C (SEQ ID NO: 50):
ATGGGATGGACCTGGATCTTTATTTTAATCCTGTCAGTTACTACAGGTGTCCA
CTCTGAGGTCCAGCTGCAGCAGTCTGGACCTGAGCTGGAGAAGCCTGGCGCTTCAGT
GAAGATATCCTGCAAGGCTTCTGGTTACTCCTTCACTGGCTACAACATGAACTGGGT
GAAACAGAGCAATGGAAAGAGCCTTGAGTGGATTGGAAATATTGATCCTTACTATG
GTGATACTAACTACAACCAGAAGTTCAAGGGCAAGGCCACATTGACTGTAGACAAA
TCCTCCAGCACAGCCTACATGCACCTCAAGAGCCTGACATCTGAGGACTCTGCAGTC
TATTACTGTGCAAGACCCTACGGTAGTGAGGCCTACTTTGCTTACTGGGGCCAAGGG
ACTCTGGTCACTGTCTCTGCAGCCAAAACGAAGGGCCCATCCGTCTTCCCCCTGGCG
CCCTGCTCCAGGAGCACCTCCGAGAGCACAGCCGCCCTGGGCTGCCTGGTCAAGGA
CTACTTCCCCGAACCGGTGACGGTGTCGTGGAACTCAGGCGCCCTGACCAGCGGCGT
GCACACCTTCCCGGCTGTCCTACAGTCCTCAGGACTCTACTCCCTCAGCAGCGTGGT
GACCGTGCCCTCCAGCAGCTTGGGCACGAAGACCTACACCTGCAACGTAGATCACA
AGCCCAGCAACACCAAGGTGGACAAGAGAGTTGAGTCCAAATATGGTCCCCCATGC
CCACCCTGCCCAGCACCTGAGTTCGAAGGGGGACCATCAGTCTTCCTGTTCCCCCCA
AAACCCAAGGACACTCTCATGATCTCCCGGACCCCTGAGGTCACGTGCGTGGTGGTG
GACGTGAGCCAGGAAGACCCCGAGGTCCAGTTCAACTGGTACGTGGATGGCGTGGA
GGTGCATAATGCCAAGACAAAGCCGCGGGAGGAGCAGTTCAACAGCACGTACCGTG
TGGTCAGCGTCCTCACCGTCCTGCACCAGGACTGGCTGAACGGCAAGGAGTACAAG
TGCAAGGTCTCCAACAAAGGCCTCCCGTCCTCCATCGAGAAAACCATCTCCAAAGCC
AAAGGGCAGCCCCGAGAGCCACAGGTGTACACCCTGCCCCCATCCCAGGAGGAGAT
GACCAAGAACCAGGTCAGCCTGACCTGCCTGGTCAAAGGCTTCTACCCCAGCGACA
TCGCCGTGGAGTGGGAGAGCAATGGGCAGCCGGAGAACAACTACAAGACCACGCCT
CCCGTGCTGGACTCCGACGGCTCCTTCTTCCTCTACAGCAGGCTAACCGTGGACAAG
AGCAGGTGGCAGGAGGGGAATGTCTTCTCATGCTCCGTGATGCATGAGGCTCTGCAC
AACCACTACACACAGAAGAGCCTCTCCCTGTCTCTGGGTAAAGCTAGCTGA
Anti-Dectin_1_2D8.2D4H-V-hIgG4H-C (SEQ ID NO: 115):
MGWTWIFILILSVTTGVHSEVQLQQSGPELEKPGASVKISCKASGYSFTGYNMNWVKQS
NGKSLEWIGNIDPYYGDTNYNQKFKGKATLTVDKSSSTAYMHLKSLTSEDSAVYYCAR
PYGSEAYFAYWGQGTLVTVSAAKTKGPSVFPLAPCSRSTSESTAALGCLVKDYFPEPVT
VSWNSGALTSGVHTFPAVLQSSGLYSLSSVVTVPSSSLGTKTYTCNVDHKPSNTKVDKR
VESKYGPPCPPCPAPEFEGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIEKT
ISKAKGQPREPQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSLSLGKAS
Anti-Dectin_1_2D8.2D4K-V-hIgGK-C (SEQ ID NO: 51):
ATGGTGTCCACTTCTCAGCTCCTTGGACTTTTGCTTTTCTGGACTTCAGCCTCC
AGATGTGACATTGTGATGACTCAGTCTCCAGCCACCCTGTCTGTGACTCCAGGAGAT
AGAGTCTCTCTTTCCTGCAGGGCCAGCCAGAGTATTAGCGACTACTTACACTGGTAT
CAACAAAAATCACATGAGTCTCCAAGGCTTCTCATCAAATATGCTGCCCAATCCATC
TCTGGGATCCCCTCCAGGTTCAGTGGCAGTGGATCAGGGTCAGATTTCACTCTCAGT
ATCAACGGTGTGGAACCTGAAGATGTTGGAGTGTATTACTGTCAAAATGGTCACAGC
TTTCCGTACACGTTCGGAGGGGGGACCAAGCTCGAGATCAAACGAACTGTGGCTGC
ACCATCTGTCTTCATCTTCCCGCCATCTGATGAGCAGTTGAAATCTGGAACTGCCTCT
GTTGTGTGCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGACAGCAA
GGACAGCACCTACAGCCTCAGCAGCACCCTGACGCTGAGCAAAGCAGACTACGAGA
AACACAAAGTCTATGCCTGCGAAGTCACCCATCAGGGCCTGAGCTCGCCCGTCACA
AAGAGCTTCAACAGGGGAGAGTGTTAG
Anti-Dectin_1_2D8.2D4K-V-hIgGK-C (SEQ ID NO: 116):
DIVMTQSPATLSVTPGDRVSLSCRASQSISDYLHWYQQKSHESPRLLIKYAAQSISGIPSR
FSGSGSGSDFTLSINGVEPEDVGVYYCQNGHSFPYTFGGGTKLEIKRTVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNRGEC
Anti-Langerin15B10H-LV-hIgG4H-C (SEQ ID NO: 52):
ATGGAATGGAGGATCTTTCTCTTCATCCTGTCAGGAACTGCAGGTGTCCACTC
CCAGGTTCAGCTGCGGCAGTCTGGACCTGAGCTGGTGAAGCCTGGGGCTTCAGTGA
AGATGTCCTGCAAGGCTTCTGGATACACATTTACTGACTATGTTATAAGTTGGGTGA
AGCAGAGAACTGGACAGGGCCTTGAGTGGATTGGAGATATTTATCCTGGAAGTGGT
TATTCTTTCTACAATGAGAACTTCAAGGGCAAGGCCACACTGACTGCAGACAAATCC
TCCACCACAGCCTACATGCAGCTCAGCAGCCTGACATCTGAGGACTCTGCGGTCTAT
TTCTGTGCAACCTACTATAACTACCCTTTTGCTTACTGGGGCCAAGGGACTCTGGTCA
CTGTCTCTGCAGCCAAAACAACGGGCCCATCCGTCTTCCCCCTGGCGCCCTGCTCCA
GGAGCACCTCCGAGAGCACAGCCGCCCTGGGCTGCCTGGTCAAGGACTACTTCCCC
GAACCGGTGACGGTGTCGTGGAACTCAGGCGCCCTGACCAGCGGCGTGCACACCTT
CCCGGCTGTCCTACAGTCCTCAGGACTCTACTCCCTCAGCAGCGTGGTGACCGTGCC
CTCCAGCAGCTTGGGCACGAAGACCTACACCTGCAACGTAGATCACAAGCCCAGCA
ACACCAAGGTGGACAAGAGAGTTGAGTCCAAATATGGTCCCCCATGCCCACCCTGC
CCAGCACCTGAGTTCGAAGGGGGACCATCAGTCTTCCTGTTCCCCCCAAAACCCAAG
GACACTCTCATGATCTCCCGGACCCCTGAGGTCACGTGCGTGGTGGTGGACGTGAGC
CAGGAAGACCCCGAGGTCCAGTTCAACTGGTACGTGGATGGCGTGGAGGTGCATAA
TGCCAAGACAAAGCCGCGGGAGGAGCAGTTCAACAGCACGTACCGTGTGGTCAGCG
TCCTCACCGTCCTGCACCAGGACTGGCTGAACGGCAAGGAGTACAAGTGCAAGGTC
TCCAACAAAGGCCTCCCGTCCTCCATCGAGAAAACCATCTCCAAAGCCAAAGGGCA
GCCCCGAGAGCCACAGGTGTACACCCTGCCCCCATCCCAGGAGGAGATGACCAAGA
ACCAGGTCAGCCTGACCTGCCTGGTCAAAGGCTTCTACCCCAGCGACATCGCCGTGG
AGTGGGAGAGCAATGGGCAGCCGGAGAACAACTACAAGACCACGCCTCCCGTGCTG
GACTCCGACGGCTCCTTCTTCCTCTACAGCAGGCTAACCGTGGACAAGAGCAGGTGG
CAGGAGGGGAATGTCTTCTCATGCTCCGTGATGCATGAGGCTCTGCACAACCACTAC
ACACAGAAGAGCCTCTCCCTGTCTCTGGGTAAAGCTAGCTGA
Anti-Langerin15B10H-LV-hIgG4H-C (SEQ ID NO: 117):
QVQLRQSGPELVKPGASVKMSCKASGYTFTDYVISWVKQRTGQGLEWIGDIYPGSGYS
FYNENFKGKATLTADKSSTTAYMQLSSLTSEDSAVYFCATYYNYPFAYWGQGTLVTVS
AAKTTGPSVFPLAPCSRSTSESTAALGCLVKDYFPEPVTVSWNSGALTSGVHTFPAVLQS
SGLYSLSSVVTVPSSSLGTKTYTCNVDHKPSNTKVDKRVESKYGPPCPPCPAPEFEGGPS
VFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLYSRLTVDKSR
WQEGNVFSCSVMHEALHNHYTQKSLSLSLGKAS.
Anti-Langerin15B10K-LV-hIgGK-C (SEQ ID NO: 53):
ATGAAGTTGCCTGTTAGGCTGTTGGTGCTGATGTTCTGGATTCCTGCTTCCAG
CAGTGATGTTGTGATGACCCAAACTCCACTCTCCCTGCCTGTCCGTCTTGGAGATCA
AGCCTCCATCTCTTGCAGATCTAGTCAGAGCCTTGTACACAGTAATGGAAACACCTA
TTTACATTGGTACCTGCAGAAGCCAGGCCAGTCTCCAAAGCTCCTGATCTACAAAGT
TTCCAACCGATTTTCTGGGGTCCCAGACAGGTTCAGTGGCAGTGGATCAGGGACAAA
TTTCACACTCAAGATCAGCAGAGTGGAGGCTGAGGATCTGGGACTTTATTTCTGCTC
TCAAAGTACACATGTTCCGTACACGTTCGGAGGGGGGACCAAGCTCGAGATCAAAC
GAACTGTGGCTGCACCATCTGTCTTCATCTTCCCGCCATCTGATGAGCAGTTGAAAT
CTGGAACTGCCTCTGTTGTGTGCCTGCTGAATAACTTCTATCCCAGAGAGGCCAAAG
TACAGTGGAAGGTGGATAACGCCCTCCAATCGGGTAACTCCCAGGAGAGTGTCACA
GAGCAGGACAGCAAGGACAGCACCTACAGCCTCAGCAGCACCCTGACGCTGAGCAA
AGCAGACTACGAGAAACACAAAGTCTATGCCTGCGAAGTCACCCATCAGGGCCTGA
GCTCGCCCGTCACAAAGAGCTTCAACAGGGGAGAGTGTTAG
Anti-Langerin15B10K-LV-hIgGK-C (SEQ ID NO: 118):
DVVMTQTPLSLPVRLGDQASISCRSSQSLVHSNGNTYLHWYLQKPGQSPKLLIYKVSNR
FSGVPDRFSGSGSGTNFTLKISRVEAEDLGLYFCSQSTHVPYTFGGGTKLEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
Anti-Langerin2G3H-LV-hIgG4H-C (SEQ ID NO: 54):
ATGACATTGAACATGCTGTTGGGGCTGAGGTGGGTTTTCTTTGTTGTTTTTTAT
CAAGGTGTGCATTGTGAGGTGCAGCTTGTTGAGTCTGGTGGAGGATTGGTGCAGCCT
AAAGGGTCATTGAAACTCTCATGTGCAGCCTCTGGATTAACCTTCAATATCTACGCC
ATGAACTGGGTCCGCCAGGCTCCAGGAAAGGGTTTGGAATGGGTTGCTCGCATAAG
AAATAAAAGTAATAATTATGCAACATATTATGCCGATTCAGTGAAAGACAGGTTCA
CCATCTCCAGAGATGATTCACAAAGCTTGCTCTATCTGCAAATGAACAACTTGAAAA
CTGAGGACACAGCCATGTATTACTGTGTGGGACGGGACTGGTTTGATTACTGGGGCC
AAGGGACTCTGGTCACTGTCTCTGCAGCCAAAACGAAGGGCCCATCCGTCTTCCCCC
TGGCGCCCTGCTCCAGGAGCACCTCCGAGAGCACAGCCGCCCTGGGCTGCCTGGTC
AAGGACTACTTCCCCGAACCGGTGACGGTGTCGTGGAACTCAGGCGCCCTGACCAG
CGGCGTGCACACCTTCCCGGCTGTCCTACAGTCCTCAGGACTCTACTCCCTCAGCAG
CGTGGTGACCGTGCCCTCCAGCAGCTTGGGCACGAAGACCTACACCTGCAACGTAG
ATCACAAGCCCAGCAACACCAAGGTGGACAAGAGAGTTGAGTCCAAATATGGTCCC
CCATGCCCACCCTGCCCAGCACCTGAGTTCGAAGGGGGACCATCAGTCTTCCTGTTC
CCCCCAAAACCCAAGGACACTCTCATGATCTCCCGGACCCCTGAGGTCACGTGCGTG
GTGGTGGACGTGAGCCAGGAAGACCCCGAGGTCCAGTTCAACTGGTACGTGGATGG
CGTGGAGGTGCATAATGCCAAGACAAAGCCGCGGGAGGAGCAGTTCAACAGCACGT
ACCGTGTGGTCAGCGTCCTCACCGTCCTGCACCAGGACTGGCTGAACGGCAAGGAG
TACAAGTGCAAGGTCTCCAACAAAGGCCTCCCGTCCTCCATCGAGAAAACCATCTCC
AAAGCCAAAGGGCAGCCCCGAGAGCCACAGGTGTACACCCTGCCCCCATCCCAGGA
GGAGATGACCAAGAACCAGGTCAGCCTGACCTGCCTGGTCAAAGGCTTCTACCCCA
GCGACATCGCCGTGGAGTGGGAGAGCAATGGGCAGCCGGAGAACAACTACAAGAC
CACGCCTCCCGTGCTGGACTCCGACGGCTCCTTCTTCCTCTACAGCAGGCTAACCGT
GGACAAGAGCAGGTGGCAGGAGGGGAATGTCTTCTCATGCTCCGTGATGCATGAGG
CTCTGCACAACCACTACACACAGAAGAGCCTCTCCCTGTCTCTGGGTAAAGCTAGCT
GA
Anti-Langerin2G3H-LV-hIgG4H-C (SEQ ID NO: 119):
MTLNMLLGLRWVFFVVFYQGVHCEVQLVESGGGLVQPKGSLKLSCAASGLTFN
IYAMNWVRQAPGKGLEWVARIRNKSNNYATYYADSVKDRFTISRDDSQSLLYLQMNN
LKTEDTAMYYCVGRDWFDYWGQGTLVTVSAAKTKGPSVFPLAPCSRSTSESTAALGCL
VKDYFPEPVTVSWNSGALTSGVHTFPAVLQSSGLYSLSSVVTVPSSSLGTKTYTCNVDH
KPSNTKVDKRVESKYGPPCPPCPAPEFEGGPSVFLFPPKPKDTLMISRTPEVTCVVVDVS
QEDPEVQFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPSDIAVEWESN
GQPENNYKTTPPVLDSDGSFFLYSRLTVDKSRWQEGNVFSCSVMHEALHNHYTQKSLS
LSLGKAS.
Anti-Langerin2G3L-LV-hIgGK-C (SEQ ID NO: 55):
ATGGCCTGGATTTCACTTATACTCTCTCTCCTGGCTCTCAGCTCAGGGGCCAT
TTCCCAGGCTGTTGTGACTCAGGAATCTGCACTCACCACATCACCTGGTGAAACAGT
CACACTCACTTGTCGCTCAAGTACTGGGGCTGTTACAACTAGTAACTATGCCAACTG
GGTCCAAGAAAAACCAGATCATTTATTCACTGGTCTAATAGGTGGTACCAACAACCG
AGTTTCAGGTGTTCCTGCCAGATTCTCAGGCTCCCTGATTGGAGACAAGGCTGCCCT
CACCATCACAGGGGCACAGACTGAGGATGAGGCAATATATTTCTGTGCTCTATGGTA
CAGCAACCATTGGGTGTTCGGTGGAGGAACCAAACTCGAGATCAAACGAACTGTGG
CTGCACCATCTGTCTTCATCTTCCCGCCATCTGATGAGCAGTTGAAATCTGGAACTGC
CTCTGTTGTGTGCCTGCTGAATAACTTCTATCCCAGAGAGGCCAAAGTACAGTGGAA
GGTGGATAACGCCCTCCAATCGGGTAACTCCCAGGAGAGTGTCACAGAGCAGGACA
GCAAGGACAGCACCTACAGCCTCAGCAGCACCCTGACGCTGAGCAAAGCAGACTAC
GAGAAACACAAAGTCTATGCCTGCGAAGTCACCCATCAGGGCCTGAGCTCGCCCGT
CACAAAGAGCTTCAACAGGGGAGAGTGTTAG
Anti-Langerin2G3L-LV-hIgGK-C (SEQ ID NO: 120):
MAWISLILSLLALSSGAISQAVVTQESALTTSPGETVTLTCRSSTGAVTTSNYANW
VQEKPDHLFTGLIGGTNNRVSGVPARFSGSLIGDKAALTITGAQTEDEAIYFCALWYSNH
WVFGGGTKLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
Anti-Lox_1_10F9H-LV-hIgG4H-C (SEQ ID NO: 56):
ATGGAATGGACCTGGGTCTTTCTCTTCCTCCTGTCAGTAACTGCAGGTGTCCA
CTCCCAGGTTCAGCTGCAGCAGTCTGGAGCTGAGCTGATGAAGCCTGGGGCCTCAGT
GAAGATATCCTGCAAGGCTACTGGCTACACATTCGGTAGCTACTGGATAGAGTGGGT
AAAGCAGAGGCCTGGACATGGCCTTGAGTGGATTGGAGAGATTTTACCTGGAAGTG
GTAATACTAACTACAATGAGAACTTCAAGGGCAAGGCCACATTCACTGCAGATACA
TCCTCCAACACAGCCTACATGCAACTCACCAGTCTGACATCTGAGGACTCTGCCGTC
TATTACTGTGCTAGGGCGGGGATTTATTGGGGCCAAGGGACTCTGGTCACTGTCTCT
GCAGCCAAAACGAAGGGCCCATCCGTCTTCCCCCTGGCGCCCTGCTCCAGGAGCAC
CTCCGAGAGCACAGCCGCCCTGGGCTGCCTGGTCAAGGACTACTTCCCCGAACCGGT
GACGGTGTCGTGGAACTCAGGCGCCCTGACCAGCGGCGTGCACACCTTCCCGGCTGT
CCTACAGTCCTCAGGACTCTACTCCCTCAGCAGCGTGGTGACCGTGCCCTCCAGCAG
CTTGGGCACGAAGACCTACACCTGCAACGTAGATCACAAGCCCAGCAACACCAAGG
TGGACAAGAGAGTTGAGTCCAAATATGGTCCCCCATGCCCACCCTGCCCAGCACCTG
AGTTCGAAGGGGGACCATCAGTCTTCCTGTTCCCCCCAAAACCCAAGGACACTCTCA
TGATCTCCCGGACCCCTGAGGTCACGTGCGTGGTGGTGGACGTGAGCCAGGAAGAC
CCCGAGGTCCAGTTCAACTGGTACGTGGATGGCGTGGAGGTGCATAATGCCAAGAC
AAAGCCGCGGGAGGAGCAGTTCAACAGCACGTACCGTGTGGTCAGCGTCCTCACCG
TCCTGCACCAGGACTGGCTGAACGGCAAGGAGTACAAGTGCAAGGTCTCCAACAAA
GGCCTCCCGTCCTCCATCGAGAAAACCATCTCCAAAGCCAAAGGGCAGCCCCGAGA
GCCACAGGTGTACACCCTGCCCCCATCCCAGGAGGAGATGACCAAGAACCAGGTCA
GCCTGACCTGCCTGGTCAAAGGCTTCTACCCCAGCGACATCGCCGTGGAGTGGGAG
AGCAATGGGCAGCCGGAGAACAACTACAAGACCACGCCTCCCGTGCTGGACTCCGA
CGGCTCCTTCTTCCTCTACAGCAGGCTAACCGTGGACAAGAGCAGGTGGCAGGAGG
GGAATGTCTTCTCATGCTCCGTGATGCATGAGGCTCTGCACAACCACTACACACAGA
AGAGCCTCTCCCTGTCTCTGGGTAAAGCTAGCTGA
Anti-Lox_1_10F9H-LV-hIgG4H-C (SEQ ID NO: 121):
MEWTWVFLFLLSVTAGVHSQVQLQQSGAELMKPGASVKISCKATGYTFGSYWI
EWVKQRPGHGLEWIGEILPGSGNTNYNENFKGKATFTADTSSNTAYMQLTSLTSEDSAV
YYCARAGIYWGQGTLVTVSAAKTKGPSVFPLAPCSRSTSESTAALGCLVKDYFPEPVTV
SWNSGALTSGVHTFPAVLQSSGLYSLSSVVTVPSSSLGTKTYTCNVDHKPSNTKVDKRV
ESKYGPPCPPCPAPEFEGGPSVFLFPPKPKDTLMISRTPEVTCVVVDVSQEDPEVQFNWY
VDGVEVHNAKTKPREEQFNSTYRVVSVLTVLHQDWLNGKEYKCKVSNKGLPSSIEKTIS
KAKGQPREPQVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSLGKAS
Anti-Lox_1_10F9K-LV-hIgGK-C (SEQ ID NO: 57):
ATGGAGAAAGACACACTCCTGCTATGGGTCCTGCTTCTCTGGGTTCCAGGTTC
CACAGGTGACATTGTGCTGACCCAATCTCCAGCTTTTTTGGCTGTGTCTCTAGGGCA
GAGGGCCACCATCTCCTGCAGAGCCAGCGAAAGTGTTGATAATTATGGCATTAGTTT
TATGAACTGGTTCCAACAGAAACCAGGACAGCCACCCAAACTCCTCATCTATGTTGC
ATCCAAGCAAGGATCCGGGGTCCCTGCCAGGTTTAGTGGCAGTGGGTCTGGGACAG
ACTTCAGCCTCAACATCCATCCTATGGAGGAGGATGATACTGCAATGTATTTCTGTC
AGCAAAGTAAGGAGGTTCCTCGGACGTTCGGTGGAGGCACCAAGCTCGAGATCAAA
CGAACTGTGGCTGCACCATCTGTCTTCATCTTCCCGCCATCTGATGAGCAGTTGAAA
TCTGGAACTGCCTCTGTTGTGTGCCTGCTGAATAACTTCTATCCCAGAGAGGCCAAA
GTACAGTGGAAGGTGGATAACGCCCTCCAATCGGGTAACTCCCAGGAGAGTGTCAC
AGAGCAGGACAGCAAGGACAGCACCTACAGCCTCAGCAGCACCCTGACGCTGAGCA
AAGCAGACTACGAGAAACACAAAGTCTATGCCTGCGAAGTCACCCATCAGGGCCTG
AGCTCGCCCGTCACAAAGAGCTTCAACAGGGGAGAGTGTTAG
Anti-Lox_1_10F9K-LV-hIgGK-C (SEQ ID NO: 122):
MEKDTLLLWVLLLWVPGSTGDIVLTQSPAFLAVSLGQRATISCRASESVDNYGIS
FMNWFQQKPGQPPKLLIYVASKQGSGVPARFSGSGSGTDFSLNIHPMEEDDTAMYFCQ
QSKEVPRTFGGGTKLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKS
FNRGEC
Anti-LOX-111C8H-LV-hIgG4H-C (SEQ ID NO: 58):
ATGGAATGTAACTGGATACTTCCTTTTATTCTGTCGGTAACTTCAGGGGTCTA
CTCAGAGGTTCAGCTCCAGCAGTCTGGGACTGTGCTGGCAAGGCCTGGGGCTTCAGT
GAAGATGTCCTGCAAGGCTTCTGGCTACACCTTTACCAGCTACTGGATGCACTGGGT
AAAACAGAGGCCTGGACAGGGTCTGGAATGGATTGGCGCTATTTATCCTGGAAATA
GTGATACTACCTACAACCAGAAGTTCAAGGGCAAGGCCAAACTGACTGCAGTCACA
TCCACCAGCACTGCCTACATGGAGCTCAGCAGCCTGACAAATGAGGACTCTGCGGT
CTATTACTGTACACCTACTTACTACTTTGACTACTGGGGCCAAGGCACCTCTCTCACA
GTCTCCTCAGCCAAAACGAAGGGCCCATCCGTCTTCCCCCTGGCGCCCTGCTCCAGG
AGCACCTCCGAGAGCACAGCCGCCCTGGGCTGCCTGGTCAAGGACTACTTCCCCGA
ACCGGTGACGGTGTCGTGGAACTCAGGCGCCCTGACCAGCGGCGTGCACACCTTCC
CGGCTGTCCTACAGTCCTCAGGACTCTACTCCCTCAGCAGCGTGGTGACCGTGCCCT
CCAGCAGCTTGGGCACGAAGACCTACACCTGCAACGTAGATCACAAGCCCAGCAAC
ACCAAGGTGGACAAGAGAGTTGAGTCCAAATATGGTCCCCCATGCCCACCCTGCCC
AGCACCTGAGTTCGAAGGGGGACCATCAGTCTTCCTGTTCCCCCCAAAACCCAAGG
ACACTCTCATGATCTCCCGGACCCCTGAGGTCACGTGCGTGGTGGTGGACGTGAGCC
AGGAAGACCCCGAGGTCCAGTTCAACTGGTACGTGGATGGCGTGGAGGTGCATAAT
GCCAAGACAAAGCCGCGGGAGGAGCAGTTCAACAGCACGTACCGTGTGGTCAGCGT
CCTCACCGTCCTGCACCAGGACTGGCTGAACGGCAAGGAGTACAAGTGCAAGGTCT
CCAACAAAGGCCTCCCGTCCTCCATCGAGAAAACCATCTCCAAAGCCAAAGGGCAG
CCCCGAGAGCCACAGGTGTACACCCTGCCCCCATCCCAGGAGGAGATGACCAAGAA
CCAGGTCAGCCTGACCTGCCTGGTCAAAGGCTTCTACCCCAGCGACATCGCCGTGGA
GTGGGAGAGCAATGGGCAGCCGGAGAACAACTACAAGACCACGCCTCCCGTGCTGG
ACTCCGACGGCTCCTTCTTCCTCTACAGCAGGCTAACCGTGGACAAGAGCAGGTGGC
AGGAGGGGAATGTCTTCTCATGCTCCGTGATGCATGAGGCTCTGCACAACCACTACA
CACAGAAGAGCCTCTCCCTGTCTCTGGGTAAAGCTAGCTGA
Anti-LOX-111C8H-LV-hIgG4H-C (SEQ ID NO: 123):
MECNWILPFILSVTSGVYSEVQLQQSGTVLARPGASVKMSCKASGYTFTSYWMH
WVKQRPGQGLEWIGAIYPGNSDTTYNQKFKGKAKLTAVTSTSTAYMELSSLTNEDSAV
YYCTPTYYFDYWGQGTSLTVSSAKTKGPSVFPLAPCSRSTSESTAALGCLVKDYFPEPVT
VSWNSGALTSGVHTFPAVLQSSGLYSLSSVVTVPSSSLGTKTYTCNVDHKPSNTKVDKR
VESKYGPPCPPCPAPEFEGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIEKT
ISKAKGQPREPQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSLSLGKAS
Anti-LOX-111C8K-LV-hIgGK-C (SEQ ID NO: 59):
ATGAGTCCTGCCCAATTCCTGTTTCTGTTAGTGCTCTGGATTCGGGAAACCAA
CGGTGATGTTGTGATGACCCAGACTCCACTCACTTTGTCGGTTACCATTGGACAACC
AGCCTCCATCTCTTGCAAGTCAAGTCAGAGCCTCTTAGATAGTGATGGAAAGACATA
TTTGAATTGGTTCTTACAGAGGCCAGGCCAGTCTCCAAAGCGCCTAATCTATCTGGT
GTCTAAACTGGACTCTGGAGTCCCTGACAGGTTCACTGGCAGTGGATCAGGGACAG
ATTTCACACTGAAAATCAGCAGAGTGGAGGCTGAGGATTTGGGAGTTTATTATTGCT
GGCAAGGTACACATTTTCCGTGGACGTTCGGTGGAGGCACCAAGCTCGAGATCAAA
CGAACTGTGGCTGCACCATCTGTCTTCATCTTCCCGCCATCTGATGAGCAGTTGAAA
TCTGGAACTGCCTCTGTTGTGTGCCTGCTGAATAACTTCTATCCCAGAGAGGCCAAA
GTACAGTGGAAGGTGGATAACGCCCTCCAATCGGGTAACTCCCAGGAGAGTGTCAC
AGAGCAGGACAGCAAGGACAGCACCTACAGCCTCAGCAGCACCCTGACGCTGAGCA
AAGCAGACTACGAGAAACACAAAGTCTATGCCTGCGAAGTCACCCATCAGGGCCTG
AGCTCGCCCGTCACAAAGAGCTTCAACAGGGGAGAGTGTTAG
Anti-LOX-111C8K-LV-hIgGK-C (SEQ ID NO: 124):
MSPAQFLFLLVLWIRETNGDVVMTQTPLTLSVTIGQPASISCKSSQSLLDSDGKTY
LNWFLQRPGQSPKRLIYLVSKLDSGVPDRFTGSGSGTDFTLKISRVEAEDLGVYYCWQG
THFPWTFGGGTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSF
NRGEC
Anti-LOX-115C4H-LV-hIgG4H-C (SEQ ID NO: 60):
ATGGGAGGGATCTGGATCTTTCTCTTCCTCCTGTCAGGAACTGCAGGTGCCCA
CTCTGAGATCCAGCTGCAGCAGACTGGACCTGAGCTGGTGAAGCCTGGGGCTTCAGT
GAAGATATCCTGCAAGGCTTCTGGTTATCCATTCACTGACTACATCATGGTCTGGGT
GAAGCAGAGCCATGGAAAGAGCCTTGAGTGGATTGGAAATATTAGTCCTTACTATG
GTACTACTAACTACAATCTGAAGTTCAAGGGCAAGGCCACATTGACTGTAGACAAA
TCTTCCAGCACAGCCTACATGCAGCTCAACAGTCTGACATCTGAGGACTCTGCAGTC
TATTACTGTGCAAGATCCCCTAACTGGGACGGGGCCTGGTTTGCTCACTGGGGCCAA
GGGGCTCTGGTCACTGTCTCTGCAGCCAAAACAAAGGGCCCATCCGTCTTCCCCCTG
GCGCCCTGCTCCAGGAGCACCTCCGAGAGCACAGCCGCCCTGGGCTGCCTGGTCAA
GGACTACTTCCCCGAACCGGTGACGGTGTCGTGGAACTCAGGCGCCCTGACCAGCG
GCGTGCACACCTTCCCGGCTGTCCTACAGTCCTCAGGACTCTACTCCCTCAGCAGCG
TGGTGACCGTGCCCTCCAGCAGCTTGGGCACGAAGACCTACACCTGCAACGTAGAT
CACAAGCCCAGCAACACCAAGGTGGACAAGAGAGTTGAGTCCAAATATGGTCCCCC
ATGCCCACCCTGCCCAGCACCTGAGTTCGAAGGGGGACCATCAGTCTTCCTGTTCCC
CCCAAAACCCAAGGACACTCTCATGATCTCCCGGACCCCTGAGGTCACGTGCGTGGT
GGTGGACGTGAGCCAGGAAGACCCCGAGGTCCAGTTCAACTGGTACGTGGATGGCG
TGGAGGTGCATAATGCCAAGACAAAGCCGCGGGAGGAGCAGTTCAACAGCACGTAC
CGTGTGGTCAGCGTCCTCACCGTCCTGCACCAGGACTGGCTGAACGGCAAGGAGTA
CAAGTGCAAGGTCTCCAACAAAGGCCTCCCGTCCTCCATCGAGAAAACCATCTCCA
AAGCCAAAGGGCAGCCCCGAGAGCCACAGGTGTACACCCTGCCCCCATCCCAGGAG
GAGATGACCAAGAACCAGGTCAGCCTGACCTGCCTGGTCAAAGGCTTCTACCCCAG
CGACATCGCCGTGGAGTGGGAGAGCAATGGGCAGCCGGAGAACAACTACAAGACC
ACGCCTCCCGTGCTGGACTCCGACGGCTCCTTCTTCCTCTACAGCAGGCTAACCGTG
GACAAGAGCAGGTGGCAGGAGGGGAATGTCTTCTCATGCTCCGTGATGCATGAGGC
TCTGCACAACCACTACACACAGAAGAGCCTCTCCCTGTCTCTGGGTAAAGCTAGCTG
ATTAATTAA
Anti-LOX-115C4H-LV-hIgG4H-C (SEQ ID NO: 125):
MGGIWIFLFLLSGTAGAHSEIQLQQTGPELVKPGASVKISCKASGYPFTDYIMVW
VKQSHGKSLEWIGNISPYYGTTNYNLKFKGKATLTVDKSSSTAYMQLNSLTSEDSAVYY
CARSPNWDGAWFAHWGQGALVTVSAAKTKGPSVFPLAPCSRSTSESTAALGCLVKDYF
PEPVTVSWNSGALTSGVHTFPAVLQSSGLYSLSSVVTVPSSSLGTKTYTCNVDHKPSNTK
VDKRVESKYGPPCPPCPAPEFEGGPSVFLFPPKPKDTLMISRTPEVTCVVVDVSQEDPEV
QFNWYVDGVEVHNAKTKPREEQFNSTYRVVSVLTVLHQDWLNGKEYKCKVSNKGLPS
SIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGKAS
Anti-LOX-115C4K-LV-hIgGK-C (SEQ ID NO: 61):
ATGGAGACAGACACAATCCTGCTATGGGTGCTGCTGCTCTGGGTTCCAGGCT
CCACTGGTGACATTGTGCTGACCCAATCTCCAGCTTCTTTGGCTGTGTCTCTAGGGCA
GAGGGCCACCATCTCCTGCAAGGCCAGCCAAAGTGTTGATTATGATGGTGATAGTTA
TATGAACTGGTTCCAACAGAAACCAGGACAGCCACCCAAACTCCTCATCTATGCTGC
ATCCAATCTAGAATCTGGGATCCCAGCCAGGTTTAGTGGCAGTGGGTCTGGGACAG
ACTTCACCCTCAACATCCATCCTGTGGAGGAGGAGGATGCTGCAACCTATTACTGTC
AGCAAAGTAATGAGGATCCATTCACGTTCGGCTCGGGGACAAAGCTCGAGATCAAA
CGAACTGTGGCTGCACCATCTGTCTTCATCTTCCCGCCATCTGATGAGCAGTTGAAA
TCTGGAACTGCCTCTGTTGTGTGCCTGCTGAATAACTTCTATCCCAGAGAGGCCAAA
GTACAGTGGAAGGTGGATAACGCCCTCCAATCGGGTAACTCCCAGGAGAGTGTCAC
AGAGCAGGACAGCAAGGACAGCACCTACAGCCTCAGCAGCACCCTGACGCTGAGCA
AAGCAGACTACGAGAAACACAAAGTCTATGCCTGCGAAGTCACCCATCAGGGCCTG
AGCTCGCCCGTCACAAAGAGCTTCAACAGGGGAGAGTGTTAG
Anti-LOX-115C4K-LV-hIgGK-C (SEQ ID NO: 126):
METDTILLWVLLLWVPGSTGDIVLTQSPASLAVSLGQRATISCKASQSVDYDGDS
YMNWFQQKPGQPPKLLIYAASNLESGIPARFSGSGSGTDFTLNIHPVEEEDAATYYCQQS
NEDPFTFGSGTKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSPVTKSFN
RGEC
Anti-Marco_10B7.3G4H-LV-hIgG4H-C (SEQ ID NO: 62):
ATGGCTGTCCTGGGGCTGCTTCTCTGCCTGGTGACGTTCCCAAGCTGTGTCCT
GTCCCAGGTGCAGCTGAAGGAGTCAGGACCTGGCCTGGTGGCACCCTCACAGAGCC
TGTCCATCACATGCACTGTCTCTGGGTTCTCATTATCCAGATATAGTGTATTTTGGGT
TCGCCAGCCTCCAGGAAAGGGTCTGGAGTGGCTGGGATTGATATGGGGTGGTGGAA
GCACAGACTATAATTCAGCTCTCAAATCCAGACTGAGCATCAGCAAGGACAACTCC
AAGAGCCAAGTTTTCTTAAAAATGAACAGTCTGCAAACTGATGACACAGCCATGTA
CTACTGTGCCAGAATCTACTTTGATTACGACGGGGCTATGGACTACTGGGGTCAAGG
AACCTCAGTCACCGTCTCCTCAGCCAAAACAACGGGCCCATCCGTCTTCCCCCTGGC
GCCCTGCTCCAGGAGCACCTCCGAGAGCACAGCCGCCCTGGGCTGCCTGGTCAAGG
ACTACTTCCCCGAACCGGTGACGGTGTCGTGGAACTCAGGCGCCCTGACCAGCGGC
GTGCACACCTTCCCGGCTGTCCTACAGTCCTCAGGACTCTACTCCCTCAGCAGCGTG
GTGACCGTGCCCTCCAGCAGCTTGGGCACGAAGACCTACACCTGCAACGTAGATCA
CAAGCCCAGCAACACCAAGGTGGACAAGAGAGTTGAGTCCAAATATGGTCCCCCAT
GCCCACCCTGCCCAGCACCTGAGTTCGAAGGGGGACCATCAGTCTTCCTGTTCCCCC
CAAAACCCAAGGACACTCTCATGATCTCCCGGACCCCTGAGGTCACGTGCGTGGTG
GTGGACGTGAGCCAGGAAGACCCCGAGGTCCAGTTCAACTGGTACGTGGATGGCGT
GGAGGTGCATAATGCCAAGACAAAGCCGCGGGAGGAGCAGTTCAACAGCACGTAC
CGTGTGGTCAGCGTCCTCACCGTCCTGCACCAGGACTGGCTGAACGGCAAGGAGTA
CAAGTGCAAGGTCTCCAACAAAGGCCTCCCGTCCTCCATCGAGAAAACCATCTCCA
AAGCCAAAGGGCAGCCCCGAGAGCCACAGGTGTACACCCTGCCCCCATCCCAGGAG
GAGATGACCAAGAACCAGGTCAGCCTGACCTGCCTGGTCAAAGGCTTCTACCCCAG
CGACATCGCCGTGGAGTGGGAGAGCAATGGGCAGCCGGAGAACAACTACAAGACC
ACGCCTCCCGTGCTGGACTCCGACGGCTCCTTCTTCCTCTACAGCAGGCTAACCGTG
GACAAGAGCAGGTGGCAGGAGGGGAATGTCTTCTCATGCTCCGTGATGCATGAGGC
TCTGCACAACCACTACACACAGAAGAGCCTCTCCCTGTCTCTGGGTAAAGCTAGCTG
Anti-Marco_10B7.3G4H-LV-hIgG4H-C (SEQ ID NO: 127):
MAVLGLLLCLVTFPSCVLSQVQLKESGPGLVAPSQSLSITCTVSGFSLSRYSVFW
VRQPPGKGLEWLGLIWGGGSTDYNSALKSRLSISKDNSKSQVFLKMNSLQTDDTAMYY
CARIYFDYDGAMDYWGQGTSVTVSSAKTTGPSVFPLAPCSRSTSESTAALGCLVKDYFP
EPVTVSWNSGALTSGVHTFPAVLQSSGLYSLSSVVTVPSSSLGTKTYTCNVDHKPSNTK
VDKRVESKYGPPCPPCPAPEFEGGPSVFLFPPKPKDTLMISRTPEVTCVVVDVSQEDPEV
QFNWYVDGVEVHNAKTKPREEQFNSTYRVVSVLTVLHQDWLNGKEYKCKVSNKGLPS
SIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGKAS
AntiMarco_10B7.3G4K_H-V-hIgGK-C (SEQ ID NO: 63):
ATGCATCGCACCAGCATGGGCATCAAGATGGAGTCACGGATTCAGGCATTTG
TATTCGTGTTTCTCTGGTTGTCTGGTGTTGGCGGAGACATTGTGATGACCCAGTCTCA
CAAATTCATGTCCACATCAGTAGGAGACAGGGTCAGCGTCACCTGCAAGGCCAGTC
AGGATGTGACTTCTGCTGTAGCCTGGTATCAACAAAAACCAGGGCAATCTCCTAAAC
TACTGATTTACTGGGCATCCACCCGGCACACTGGAGTCCCTGATCGCTTCACAGGCA
GTGGATCTGGGACAGATTATACTCTCACCATCAGCAGTGGGCAGGCTGAAGACCTG
GCACTTTATTACTGTCACCAATATTATAGCGCTCCTCGGACGTTCGGTGGAGGCACC
AAGCTCGAGATCAAACGAACTGTGGCTGCACCATCTGTCTTCATCTTCCCGCCATCT
GATGAGCAGTTGAAATCTGGAACTGCCTCTGTTGTGTGCCTGCTGAATAACTTCTAT
CCCAGAGAGGCCAAAGTACAGTGGAAGGTGGATAACGCCCTCCAATCGGGTAACTC
CCAGGAGAGTGTCACAGAGCAGGACAGCAAGGACAGCACCTACAGCCTCAGCAGC
ACCCTGACGCTGAGCAAAGCAGACTACGAGAAACACAAAGTCTATGCCTGCGAAGT
CACCCATCAGGGCCTGAGCTCGCCCGTCACAAAGAGCTTCAACAGGGGAGAGTGTT
AG
AntiMarco_10B7.3G4K_H-V-hIgGK-C (SEQ ID NO: 128):
MHRTSMGIKMESRIQAFVFVFLWLSGVGGDIVMTQSHKFMSTSVGDRVSVTCK
ASQDVTSAVAWYQQKPGQSPKLLIYWASTRHTGVPDRFTGSGSGTDYTLTISSGQAEDL
ALYYCHQYYSAPRTFGGGTKLEIKRTVAAPSVFIFPPSDEQLKSGTASVVCLLNNFYPRE
AKVQWKVDNALQSGNSQESVTEQDSKDSTYSLSSTLTLSKADYEKHKVYACEVTHQGL
SSPVTKSFNRGEC
Anti-Marco_11A8.3C9_H-V-hIgG4H-C (SEQ ID NO: 64):
ATGGAATGGAACTGGGTCGTTCTCTTCCTCCTGTCATTAACTGCAGGTGTCTA
TGCCCAGGGTCAGATGCAGCAGTCTGGAGCTGAGCTGGTGAAGCCTGGGGCTTCAG
TGAAGCTGTCCTGCAAGACTTCTGGCTTCACCTTCAGCAGTAACTATATAAGTTGGT
TGAAGCAAAAGCCTGGACAGAGTCTTGAGTGGATTGCATGGATTTATGCTGGAACT
GGTGGTATTACCTATAATCAGAAGTTCAGAGGCAGGGCCCAACTGACTGTAGACAC
ATCCTCCAGCACAGCCTACATGCAGTTCAGCAGCCTGACAACTGATGACTCTGCCAT
CTATTACTGTGCAAGACACGTGAGGGGTTACCATCCTATGGACTACTGGGGTCAAGG
AACCTCAGTCACCGTCTCCTCAGCCAAAACGAAGGGCCCATCCGTCTTCCCCCTGGC
GCCCTGCTCCAGGAGCACCTCCGAGAGCACAGCCGCCCTGGGCTGCCTGGTCAAGG
ACTACTTCCCCGAACCGGTGACGGTGTCGTGGAACTCAGGCGCCCTGACCAGCGGC
GTGCACACCTTCCCGGCTGTCCTACAGTCCTCAGGACTCTACTCCCTCAGCAGCGTG
GTGACCGTGCCCTCCAGCAGCTTGGGCACGAAGACCTACACCTGCAACGTAGATCA
CAAGCCCAGCAACACCAAGGTGGACAAGAGAGTTGAGTCCAAATATGGTCCCCCAT
GCCCACCCTGCCCAGCACCTGAGTTCGAAGGGGGACCATCAGTCTTCCTGTTCCCCC
CAAAACCCAAGGACACTCTCATGATCTCCCGGACCCCTGAGGTCACGTGCGTGGTG
GTGGACGTGAGCCAGGAAGACCCCGAGGTCCAGTTCAACTGGTACGTGGATGGCGT
GGAGGTGCATAATGCCAAGACAAAGCCGCGGGAGGAGCAGTTCAACAGCACGTAC
CGTGTGGTCAGCGTCCTCACCGTCCTGCACCAGGACTGGCTGAACGGCAAGGAGTA
CAAGTGCAAGGTCTCCAACAAAGGCCTCCCGTCCTCCATCGAGAAAACCATCTCCA
AAGCCAAAGGGCAGCCCCGAGAGCCACAGGTGTACACCCTGCCCCCATCCCAGGAG
GAGATGACCAAGAACCAGGTCAGCCTGACCTGCCTGGTCAAAGGCTTCTACCCCAG
CGACATCGCCGTGGAGTGGGAGAGCAATGGGCAGCCGGAGAACAACTACAAGACC
ACGCCTCCCGTGCTGGACTCCGACGGCTCCTTCTTCCTCTACAGCAGGCTAACCGTG
GACAAGAGCAGGTGGCAGGAGGGGAATGTCTTCTCATGCTCCGTGATGCATGAGGC
TCTGCACAACCACTACACACAGAAGAGCCTCTCCCTGTCTCTGGGTAAAGCTAGCTGA
Anti-Marco_11A8.3C9_H-V-hIgG4H-C (SEQ ID NO: 129):
MEWNWVVLFLLSLTAGVYAQGQMQQSGAELVKPGASVKLSCKTSGFTFSSNYI
SWLKQKPGQSLEWIAWIYAGTGGITYNQKFRGRAQLTVDTSSSTAYMQFSSLTTDDSAI
YYCARHVRGYHPMDYWGQGTSVTVSSAKTKGPSVFPLAPCSRSTSESTAALGCLVKDY
FPEPVTVSWNSGALTSGVHTFPAVLQSSGLYSLSSVVTVPSSSLGTKTYTCNVDHKPSNT
KVDKRVESKYGPPCPPCPAPEFEGGPSVFLFPPKPKDTLMISRTPEVTCVVVDVSQEDPE
VQFNWYVDGVEVHNAKTKPREEQFNSTYRVVSVLTVLHQDWLNGKEYKCKVSNKGL
PSSIEKTISKAKGQPREPQVYTLPPSQEEMTKNQVSLTCLVKGFYPSDIAVEWESNGQPE
NNYKTTPPVLDSDGSFFLYSRLTVDKSRWQEGNVFSCSVMHEALHNHYTQKSLSLSLG
KAS
Anti-Marco_11A8.3C9_H-V-hIgGK-C (SEQ ID NO: 65):
ATGGAGTCACAGACTCAGGTCTTTGTATACATGTTGCTGTGGTTGTCTGGTGT
TGATGGAGACATTGTGATGACCCAGTCTCAAAAATTCATGTCCGCATCAGTAGGGGA
CAGGGTCAGCGTCACCTGCAGGGCCAGTCAGAATGTGGTTACTAATGTAGGCTGGT
ATCAACAGAAACCAGGGCAATCTCCTAAAGTACTGATTTACTCGGCATCCTTCCGGT
ACAGTGGAGTCCCTGATCGCTTCACAGGCAGTGGATCTGGGACAGATTTCACTCTCA
CCATCACCAATGTGCAGTCTGAAGACTTGGCAGAGTATTTCTGTCAGCAATATAACA
ACTATCCGTACACGTTCGGAGGGGGGACCAAGCTCGAGATCAAACGAACTGTGGCT
GCACCATCTGTCTTCATCTTCCCGCCATCTGATGAGCAGTTGAAATCTGGAACTGCCT
CTGTTGTGTGCCTGCTGAATAACTTCTATCCCAGAGAGGCCAAAGTACAGTGGAAGG
TGGATAACGCCCTCCAATCGGGTAACTCCCAGGAGAGTGTCACAGAGCAGGACAGC
AAGGACAGCACCTACAGCCTCAGCAGCACCCTGACGCTGAGCAAAGCAGACTACGA
GAAACACAAAGTCTATGCCTGCGAAGTCACCCATCAGGGCCTGAGCTCGCCCGTCA
CAAAGAGCTTCAACAGGGGAGAGTGTTAG
Anti-Marco_11A8.3C9_H-V-hIgGK-C (SEQ ID NO: 130):
MESQTQVFVYMLLWLSGVDGDIVMTQSQKFMSASVGDRVSVTCRASQNVVTN
VGWYQQKPGQSPKVLIYSASFRYSGVPDRFTGSGSGTDFTLTITNVQSEDLAEYFCQQY
NNYPYTFGGGTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSF
NRGEC
Anti-Marco_3H10.1F3_H-V-hIgG4H-C (SEQ ID NO: 66):
ATGGGATGGAGCTATATCATCCTCTTTTTGGTAGCAACAGCTACAGATGTCCA
CTCCCAGGTCCAACTGCAGCAGCCTGGGGCTGAACTGGTGAAGCCTGGGGCTTCAG
TGAAGCTGTCCTGCAAGGCTTCTGGCTACACCTTCACCAGCTACTGGATGCACTGGG
TGAAGCAGAGGCCTGGAGAAGGCCTTGAGTGGATTGGAGAGATTAATCCTAGCTAC
GGTCGTACTGACTACAATGGGAAGTTCAAGAACAAGGCCACACTGACTGTAGCCAA
ATCCTCCAGCACAGCCTACATGCAACTCAGCAGCCTGACATCTGAGGACTCTGCGGT
CTATTACTGTGCAAGAGGAGATTACTACGGTAGTAGCTCGTTTGCTTACTGGGGCCA
AGGGACTCTGGTCACTGTCTCTGCAGCCAAAACAAAGGGCCCATCCGTCTTCCCCCT
GGCGCCCTGCTCCAGGAGCACCTCCGAGAGCACAGCCGCCCTGGGCTGCCTGGTCA
AGGACTACTTCCCCGAACCGGTGACGGTGTCGTGGAACTCAGGCGCCCTGACCAGC
GGCGTGCACACCTTCCCGGCTGTCCTACAGTCCTCAGGACTCTACTCCCTCAGCAGC
GTGGTGACCGTGCCCTCCAGCAGCTTGGGCACGAAGACCTACACCTGCAACGTAGA
TCACAAGCCCAGCAACACCAAGGTGGACAAGAGAGTTGAGTCCAAATATGGTCCCC
CATGCCCACCCTGCCCAGCACCTGAGTTCGAAGGGGGACCATCAGTCTTCCTGTTCC
CCCCAAAACCCAAGGACACTCTCATGATCTCCCGGACCCCTGAGGTCACGTGCGTGG
TGGTGGACGTGAGCCAGGAAGACCCCGAGGTCCAGTTCAACTGGTACGTGGATGGC
GTGGAGGTGCATAATGCCAAGACAAAGCCGCGGGAGGAGCAGTTCAACAGCACGTA
CCGTGTGGTCAGCGTCCTCACCGTCCTGCACCAGGACTGGCTGAACGGCAAGGAGT
ACAAGTGCAAGGTCTCCAACAAAGGCCTCCCGTCCTCCATCGAGAAAACCATCTCC
AAAGCCAAAGGGCAGCCCCGAGAGCCACAGGTGTACACCCTGCCCCCATCCCAGGA
GGAGATGACCAAGAACCAGGTCAGCCTGACCTGCCTGGTCAAAGGCTTCTACCCCA
GCGACATCGCCGTGGAGTGGGAGAGCAATGGGCAGCCGGAGAACAACTACAAGAC
CACGCCTCCCGTGCTGGACTCCGACGGCTCCTTCTTCCTCTACAGCAGGCTAACCGT
GGACAAGAGCAGGTGGCAGGAGGGGAATGTCTTCTCATGCTCCGTGATGCATGAGG
CTCTGCACAACCACTACACACAGAAGAGCCTCTCCCTGTCTCTGGGTAAAGCTAGCT
GA
Anti-Marco_3H10.1F3_H-V-hIgG4H-C (SEQ ID NO: 131):
MGWSYIILFLVATATDVHSQVQLQQPGAELVKPGASVKLSCKASGYTFTSYWM
HWVKQRPGEGLEWIGEINPSYGRTDYNGKFKNKATLTVAKSSSTAYMQLSSLTSEDSA
VYYCARGDYYGSSSFAYWGQGTLVTVSAAKTKGPSVFPLAPCSRSTSESTAALGCLVK
DYFPEPVTVSWNSGALTSGVHTFPAVLQSSGLYSLSSVVTVPSSSLGTKTYTCNVDHKPS
NTKVDKRVESKYGPPCPPCPAPEFEGGPSVFLFPPKPKDTLMISRTPEVTCVVVDVSQED
PEVQFNWYVDGVEVHNAKTKPREEQFNSTYRVVSVLTVLHQDWLNGKEYKCKVSNK
GLPSSIEKTISKAKGQPREPQVYTLPPSQEEMTKNQVSLTCLVKGFYPSDIAVEWESNGQ
PENNYKTTPPVLDSDGSFFLYSRLTVDKSRWQEGNVFSCSVMHEALHNHYTQKSLSLSL
GKAS
Anti-Marco_3H10.1F3_K-V-hIgGK-C (SEQ ID NO: 67):
ATGGAGTCACAGACTCAGGTCTTTGTATACATGTTGCTGTGGTTGTCTGGTGT
TGATGGAGACATTGTGATGACCCAGTCTCAAAAATTCATGTCCACATCATTAGGAGA
CAGGGTCAGCGTCACCTGCAAGGCCAGTCAGAATGTGGGTACTAATGTAGCCTGGT
ATCAACAGAAACCAGGGCACTCTCCTAAAGCACTGATTTACTCGGCATCCTACCGGT
ACAGTGGAGTCCCTGATCGCTTCACAGGCAGTGGATCTGGGACAGATTTCACTCTCA
CCATCAGCAATGTGCAGTCTGAAGACTTGGCAGAGTTTTTCTGTCAGCAATATAACA
ACTATCCGTACACGTTCGGAGGGGGGACCACGCTCGAGATCAAACGAACTGTGGCT
GCACCATCTGTCTTCATCTTCCCGCCATCTGATGAGCAGTTGAAATCTGGAACTGCCT
CTGTTGTGTGCCTGCTGAATAACTTCTATCCCAGAGAGGCCAAAGTACAGTGGAAGG
TGGATAACGCCCTCCAATCGGGTAACTCCCAGGAGAGTGTCACAGAGCAGGACAGC
AAGGACAGCACCTACAGCCTCAGCAGCACCCTGACGCTGAGCAAAGCAGACTACGA
GAAACACAAAGTCTATGCCTGCGAAGTCACCCATCAGGGCCTGAGCTCGCCCGTCA
CAAAGAGCTTCAACAGGGGAGAGTGTTAG
Anti-Marco_3H10.1F3_K-V-hIgGK-C (SEQ ID NO: 132):
MESQTQVFVYMLLWLSGVDGDIVMTQSQKFMSTSLGDRVSVTCKASQNVGTN
VAWYQQKPGHSPKALIYSASYRYSGVPDRFTGSGSGTDFTLTISNVQSEDLAEFFCQQY
NNYPYTFGGGTTLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSF
NRGEC

The antigens of the present invention comprises one or more viral antigens or peptides from adenovirus, retrovirus, picornavirus, herpesvirus, rotaviruses, hantaviruses, coronavirus, togavirus, flavirvirus, rhabdovirus, paramyxovirus, orthomyxovirus, bunyavirus, arenavirus, reovirus, papilomavirus, parvovirus, poxvirus, hepadnavirus, or spongiform virus, HIV, CMV, hepatitis A, B, and C, influenza; measles, polio, smallpox, rubella; respiratory syncytial, herpes simplex, varicella zoster, Epstein-Barr, Japanese encephalitis, rabies, flu, or cold viruses. The antigen is selected from: Nef (66-97): VGFPVTPQVPLRPMTYKAAVDLSHFLKEKGGL (SEQ ID NO: 154); Nef (116-145): HTQGYFPDWQNYTPGPGVRYPLTFGWLYKL (SEQ ID NO: 155); Gag p17 (17-35): EKIRLRPGGKKKYKLKHIV (SEQ ID NO: 156); Gag p17-p24 (253-284): NPPIPVGEIYKRWIILGLNKIVRMYSPTSILD (SEQ ID NO: 157); and/or Pol 325-355 (RT 158-188) is: AIFQSSMTKILEPFRKQNPDIVIYQYMDDLY (SEQ ID NO: 158). In one aspect the said antigen is 19 to 32 residues and is selected from a cytotoxic T lymphocyte (CTL) epitope identified in the HIV-1 Nef, Gag and Env proteins presented in the context of MHC-class I molecules. In another aspect, the Ag is selected from HIV gp120, gp41, Gag, p17, p24, p2, p′7, p1, p6, Tat, Rev, PR, RT, IN, Vif, Vpr, Vpx, Vpu and Nef.

In another aspect the antigen is selected from tumor associated antigens selected from CEA, prostate specific antigen (PSA), HER-2/neu, BAGE, GAGE, MAGE 1-4, 6 and 12, MUC-related protein (Mucin) (MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67. In another aspect, the Ag is selected from tumor associated antigens comprising antigens from leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia.

The Ag is selected from at least one of:

(SEQ ID NO: 133)

MWVPVVFLTLSVTWIGAAPLILSRIVGGWECEKHSQPWQVLVASRGRAVC
GGVLVHPQWV;

(SEQ ID NO: 134)

LTAAHCIRNKSVILLGRHSLFHPEDTGQVFQVSHSFPHPLYDMSLLKNRF
LRPGDDSSHD;

(SEQ ID NO: 135)

LMLLRLSEPAELTDAVKVMDLPTQEPALGTTCYASGWGSIEPEEFLTPKK
LQCVDLHVIS;

(SEQ ID NO: 136)

NDVCAQVHPQKVTKFMLCAGRWTGGKSTCSGDSGGPLVCNGVLQGITSWG
SEPCALPERP;
or

(SEQ ID NO: 137)

SLYTKVVHYRKWIKDTIVANP.

In another aspect, the Ag is selected from at least one of:

(SEQ ID NO: 138)

IMDQVPFSV;

(SEQ ID NO: 139)

ITDQVPFSV;

(SEQ ID NO: 140)

YLEPGPVTV;

(SEQ ID NO: 141)

YLEPGPVTA;

(SEQ ID NO: 142)

KTWGQYWQV;

(SEQ ID NO: 143)

DTTEPATPTTPVTTPTTTKVPRNQDWLGVSRQLRTKAWNRQLYPEWTEAQ
RLDCWRGGQVSLKVSNDGPTLIGANASFSIALNFPGSQKVLPDGQVIWVN
NTIINGSQVWGGQPVYPQETDDACIFPDGGPCPSGSWSQKRSFVYVWKTW
GQYWQVLGGPVSGLSIGTGRAMLGTHTMEVTVYHRRGSQSYVPLAHSSSA
FTITDQVPFSVSVSQLRALDGGNKHFLRNQ;

(SEQ ID NO: 144)

PLTFALQLHDPSGYLAEADLSYTWDFGDSSGTLISRAXVVTHTYLEPGPV
TAQVVLQAAIPLTSCGSSPVPAS;

(SEQ ID NO: 145)

GTTDGHRPTAEAPNTTAGQVPTTEVVGTTPGQAPTAEPSGTTSVQVPTTE
VISTAPVQMPTAESTGMTPEKVPVSEVMGTTLAEMSTPEATGMTPAEVSI
VVLSGTTAA;

(SEQ ID NO: 146)

QVTTTEWVETTARELPIPEPEGPDASSIMSTESITGSLGPLLDGTATLRL
VKRQVPLDCVLYRYGSFSVTLDIVQ;
and

(SEQ ID NO: 147)

GIESAEILQAVPSGEGDAFELTVSCQGGLPKEACMEISSPGCQPPAQRLC
QPVLPSPACQLVLHQILKGGSGTYCLNVSLADTNSLAVVSTQLIVPGIL
LTGQEAGLGQ,
and fragments thereof.

In yet another aspect, the Ag is selected from at least one of:

(SEQ ID NO: 148)

MEMKILRALNFGLGRPLPLHFLRRASKIGEVDVEQHTLAKYLMELTML
DY;
and

(SEQ ID NO: 149)

DWLVQVQMKFRLLQETMYMTVSIIDRFMQNNCVPKK.

In another aspect, the Ag is selected from at least one of:

(SEQ ID NO: 150)

MEHQLLCCEVETIRRAYPDANLLNDRVLRAMLKAEETCAPSVSYFKCV;

(SEQ ID NO: 151)

QKEVLPSMRKIVATWMLEVCEEQKCEEEVFPLAMNYLDRFLSLEPVKKSR
LQLLGATCMFVASKMKETIPLTAEKLCIYTDNSIRPEELLQMELL;

(SEQ ID NO: 152)

LVNKLKWNLAAMTPHDFIEHFLSKMPEAEENKQIIRKHAQTFVALCATDV
KFISNPPSMV;
and

(SEQ ID NO: 153)

AGSVVAAVQGLNLRSPNNFLSYYRLTRFLSRVIKCDPDCLRACQEQIEAL
LESSLRQAQQNMDPKAAEEEEEEEEEVDLACTPTDVRDVDI,
and fragments thereof.

In another aspect, the Ag is 19 to 32 amino acids long. In another aspect, the Ag is 17 to 60 amino acids long and is selected from a cytotoxic T lymphocyte (CTL) epitope identified in PSA or cyclin 1

In another aspect, the cancer peptides are selected from tumor associated antigens selected from CEA, prostate specific antigen (PSA), HER-2/neu, BAGE, GAGE, MAGE 1-4, 6 and 12, MUC-related protein (Mucin) (MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67.

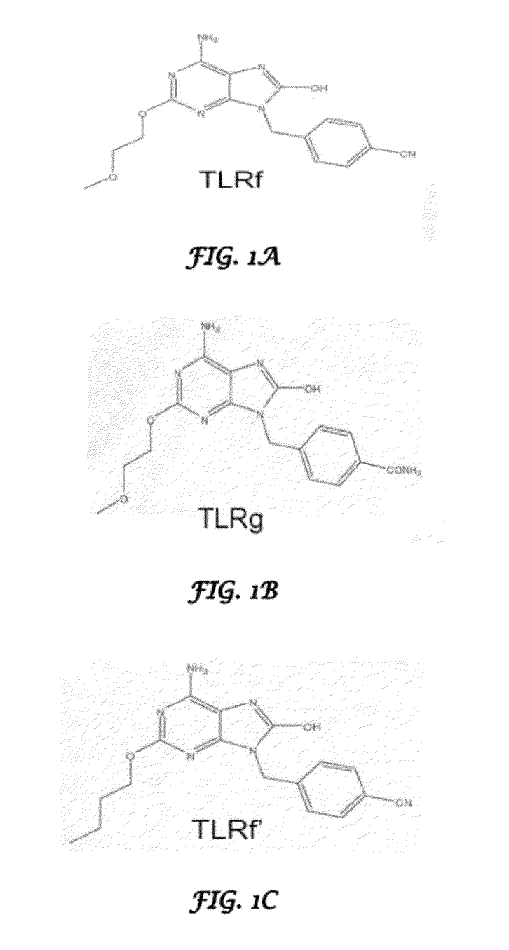

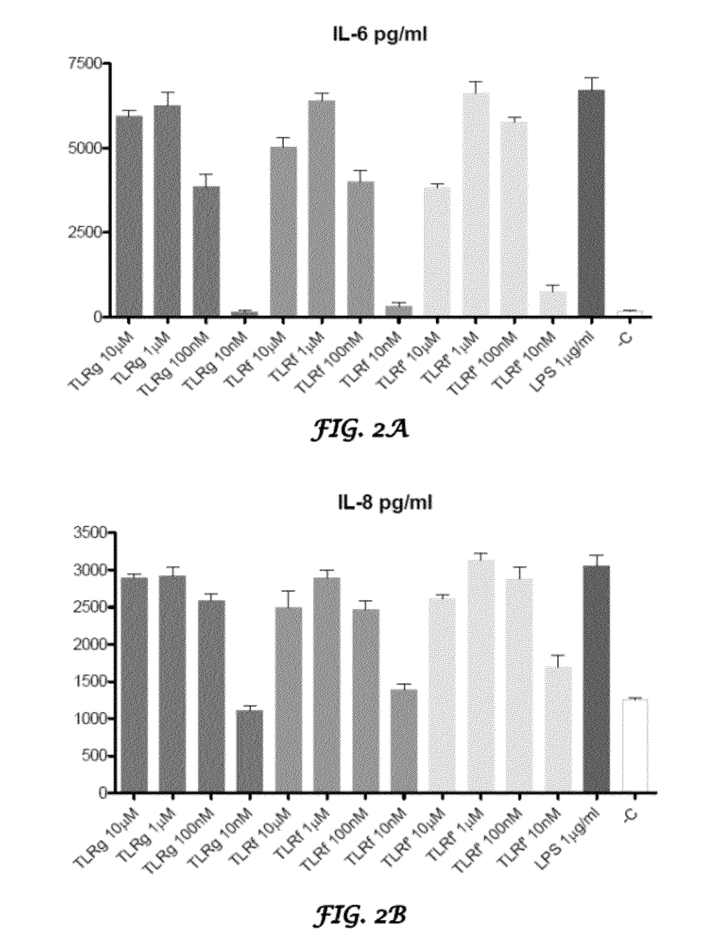

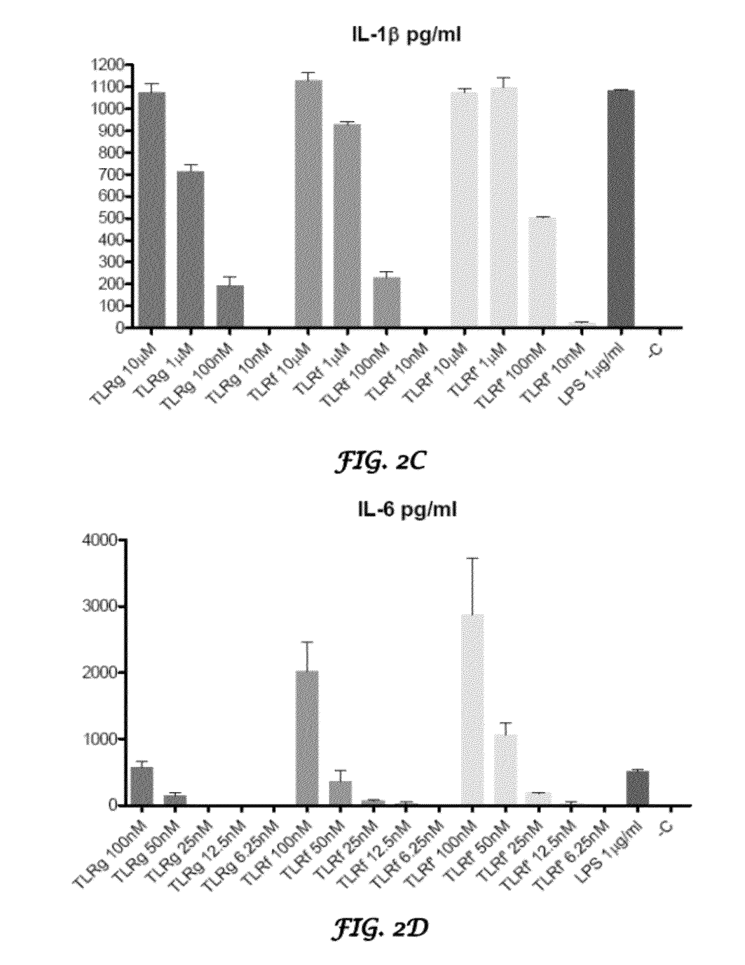

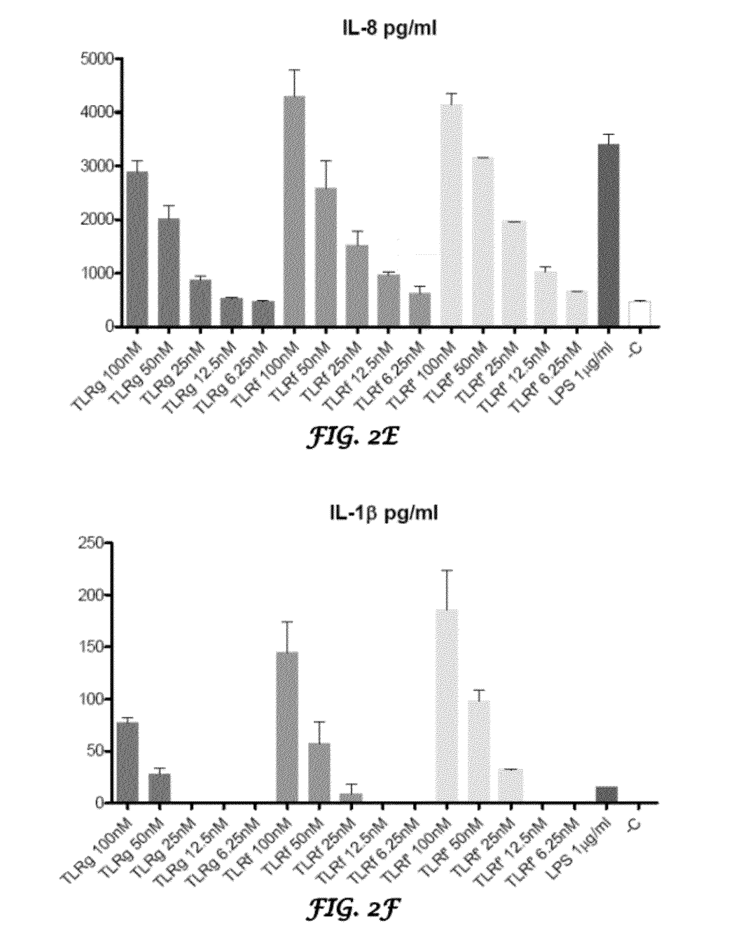

FIGS. 1A-1C show the chemical structures of TLR7L variants as described in the instant invention. FIGS. 2A-2C show the dose titration of the TLR9, TLRf, and TLRf′ variants of the instant invention at concentrations of 10 nM, 100 nM, 1 μM, and 10 μM. The activity is measured by the secretion (pg/mL) of: IL-6 (FIG. 2A), IL-8 (FIG. 2B), IL-1β (FIG. 2C). The dose titration curves indicate that the activity of the 3 different TLR7-L compounds is pretty similar. All 3 compounds are still effective at 100 nM;

FIGS. 2D-2G show the dose titration of the TLR9, TLRf, and TLRf variants of the instant invention at concentrations of 12.5 nM, 25 nM, 50 nM, and 100 nM. The activity is measured by the secretion of: IL-6 (FIG. 2D), IL-8 (FIG. 2E), IL-1β (FIG. 2F), and IL-12p40 (FIG. 2G). The dose titration curves indicate that the TLRf is marginally more effective and its activity is still detected at 25 nM;

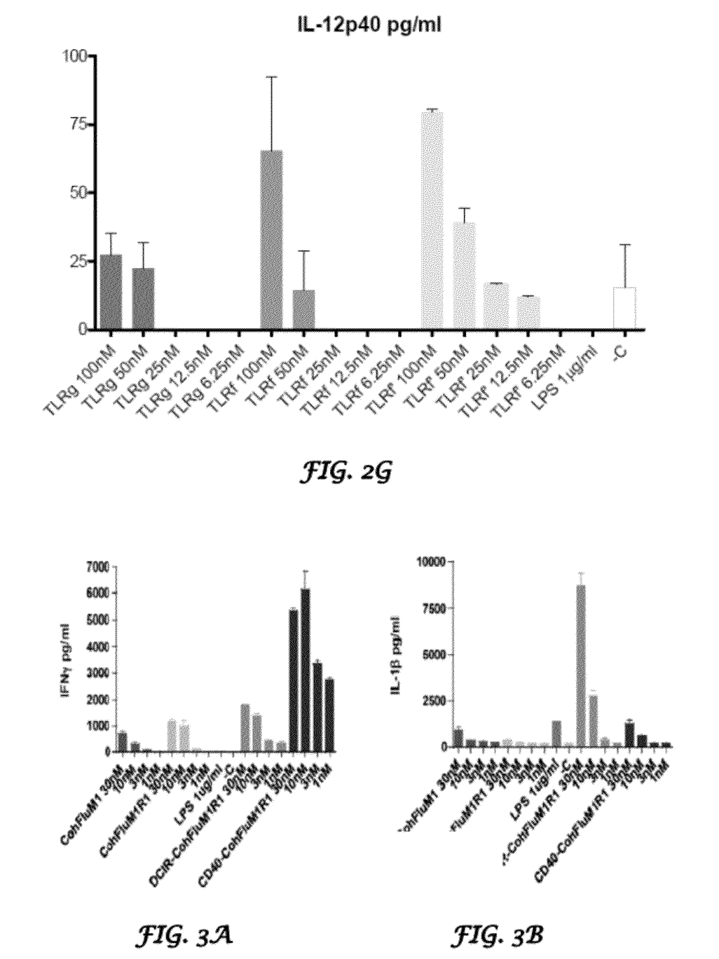

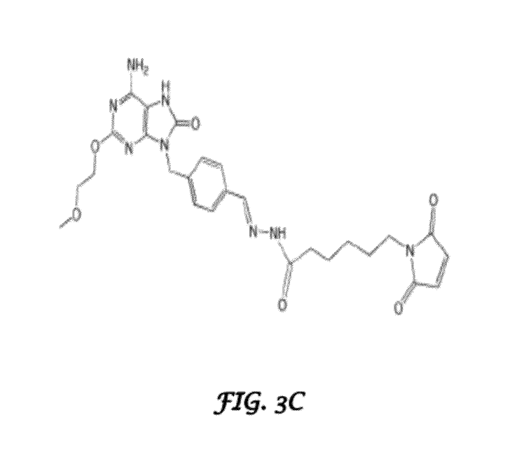

FIGS. 3A and 3B show that TLR7L linked to DC-targeting mAb potentiates and restricts adjuvant activity. The activity of the TLR7L linked to CohFluM1 is dependent on the targeting antibody. TLR7L linked to CohFluM1 or CohFluM1 alone are effective only at highest 30 nM dose. Moreover, there is a difference in potency and relative amounts of cytokines induced with anti-DCIR and anti-CD40 antibodies linked to TLR7L. FIG. 3C is a chemical structure of a TLR7 ligand according to an embodiment of the present invention.

It is contemplated that any embodiment discussed in this specification can be implemented with respect to any method, kit, reagent, or composition of the invention, and vice versa. Furthermore, compositions of the invention can be used to achieve methods of the invention.

It may be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it may be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Patent Application No. 20090004194: TLR Agonist (Flagellin)/CD40 Agonist/Antigen Protein and DNA Conjugates and use thereof for Inducing Synergistic Enhancement in Immunity.
U.S. Patent Application No. 20080220011: Use of Flagellin in Tumor Immunotherapy.
U.S. Patent Application No. 20080248068: Use of Flagellin as an Adjuvant for Vaccine.
U.S. Pat. No. 7,404,963: Flagellin-Based Adjuvants and Vaccines.

What is claimed is:

1. An adjuvant composition comprising:
an anti-dendritic cell (DC)-specific antibody or fragment thereof conjugated to a Toll-Like Receptor (TLR) agonist; and a pharmaceutically acceptable carrier, wherein the conjugate and agonist are each comprised in an amount such that, in combination with the other, are effective to produce the immune response in a human or animal subject in need of immunostimulation.

2. The composition of claim 1, wherein the DC-specific antibody or fragment is selected from an anti-DCIR, MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, or ASPGR.

3. The composition of claim 1, wherein a nucleotide sequence of the DC-specific antibody is selected from SEQ ID NOS.: 3 to 67.

4. The composition of claim 1, wherein an amino acid sequence of the DC-specific antibody is selected from SEQ ID NOS.: 68 to 132.

5. The composition of claim 1, wherein the composition further comprises antigenic peptides selected from human immunodeficiency virus (HIV) antigens and gene products selected from the group consisting of gag, pol, and env genes, the Nef protein, reverse transcriptase, string of HIV peptides (Hipo5), PSA (KLQCVDLHV)-tetramer (SEQ ID NO: 159), a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide (GILGFVFTL) tetramer (SEQ ID NO: 160), and Avian Flu (HA5-1), dockerin domain from C. thermocellum, measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens or combinations and modifications thereof.

6. The composition of claim 1, wherein the composition further comprises antigenic peptides selected from cancer peptides are selected from tumor associated antigens comprising antigens from leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia.

7. The composition of claim 1, wherein the composition further comprises antigenic peptides selected from tumor associated antigens are selected from CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67.

8. The composition of claim 1, wherein the DC-specific antibody is humanized.

9. The composition of claim 1, wherein the composition is administered to the human or animal subject by an oral route, a nasal route, topically or as an injection.

10. The composition of claim 9, wherein the injection is selected from the group consisting of subcutaneous, intravenous, intraperitoneal, intramuscular, and intravenous.

11. A vaccine composition comprising:
an antigen; and
an adjuvant, wherein the adjuvant comprises an anti-dendritic cell (DC)-specific antibody or fragment thereof conjugated to a Toll-Like Receptor (TLR) agonist; and a pharmaceutically acceptable carrier, wherein the conjugate and agonist are each comprised in an amount such that, in combination with the other, are effective to produce the immune response in a human or animal subject in need of immunostimulation.

12. The composition of claim 11, wherein the DC-specific antibody or fragment is selected from an anti-DCIR, MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, or ASPGR.

13. The composition of claim 11, wherein the composition further comprises antigenic peptides selected from human immunodeficiency virus (HIV) antigens and gene products selected from the group consisting of gag, pol, and env genes, the Nef protein, reverse transcriptase, string of HIV peptides (Hipo5), PSA (KLQCVDLHV)-tetramer (SEQ ID NO: 159), a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide (GILGFVFTL) tetramer (SEQ ID NO: 160), and Avian Flu (HA5-1), dockerin domain from C. thermocellum, measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens or combinations and modifications thereof.

14. The composition of claim 11, wherein the composition further comprises antigenic peptides selected from cancer peptides are selected from tumor associated antigens comprising antigens from leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia.

15. The composition of claim 14, wherein the composition further comprises antigenic peptides selected from tumor associated antigens are selected from CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67.

16. The composition of claim 11, wherein the DC-specific antibody is humanized.

17. The composition of claim 11, wherein the composition is administered to the human or animal subject by an oral route, a nasal route, topically or as an injection.

18. The composition of claim 17, wherein the injection is selected from the group consisting of subcutaneous, intravenous, intraperitoneal, intramuscular, and intravenous.

19. A method for increasing effectiveness of antigen presentation by an antigen presenting cell comprising:
contacting the antigen presenting cell with a composition comprising:
an antigen; and
an adjuvant, wherein the adjuvant comprises an anti-dendritic cell (DC)-specific antibody or fragment thereof conjugated to a Toll-Like Receptor (TLR) agonist; and a pharmaceutically acceptable carrier, wherein the conjugate and agonist are each comprised in an amount such that, in combination with the other, are effective to produce the immune response in a human or animal subject in need of immunostimulation.

20. The method of claim 19, wherein the DC-specific antibody or fragment is selected from an anti-DCIR, MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, or ASPGR.

21. The method of claim 19, wherein the composition further comprises antigenic peptides selected from human immunodeficiency virus (HIV) antigens and gene products selected from the group consisting of gag, pol, and env genes, the Nef protein, reverse transcriptase, string of HIV peptides (Hipo5), PSA (KLQCVDLHV)-tetramer (SEQ ID NO: 159), a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide (GILGFVFTL) tetramer (SEQ ID NO: 160), and Avian Flu (HA5-1), dockerin domain from C. thermocellum, measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens or combinations and modifications thereof.

22. The method of claim 19, wherein the composition further comprises antigenic peptides selected from cancer peptides are selected from tumor associated antigens comprising antigens from leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia.

23. The method of claim 19, wherein the composition further comprises antigenic peptides selected from tumor associated antigens are selected from CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67.

24. The method of claim 19, wherein the DC-specific antibody is humanized.

25. The method of claim 19, wherein the composition is administered to the human or animal subject by an oral route, a nasal route, topically or as an injection.

26. The method of claim 25, wherein the injection is selected from the group consisting of subcutaneous, intravenous, intraperitoneal, intramuscular, and intravenous.

27. A method for a treatment, a prophylaxis or a combination thereof against one or more cancers in a human subject comprising the steps of:
identifying the human subject in need of the treatment, the prophylaxis or a combination thereof; and
administering a vaccine composition comprising:
an antigen; and
an adjuvant, wherein the adjuvant comprises an anti-dendritic cell (DC)-specific antibody or fragment thereof conjugated to a Toll-Like Receptor (TLR) agonist; and a pharmaceutically acceptable carrier, wherein the conjugate and agonist are each comprised in an amount such that, in combination with the other, are effective to produce the immune response in a human or animal subject in need of immunostimulation.

28. The method of claim 27, wherein the DC-specific antibody or fragment is selected from an anti-DCIR, MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, or ASPGR.

29. The method of claim 27, wherein the composition further comprises antigenic peptides selected from human immunodeficiency virus (HIV) antigens and gene products selected from the group consisting of gag, pol, and env genes, the Nef protein, reverse transcriptase, string of HIV peptides (Hipo5), PSA (KLQCVDLHV)-tetramer (SEQ ID NO: 159), a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide (GILGFVFTL) tetramer (SEQ ID NO: 160), and Avian Flu (HA5-1), dockerin domain from C. thermocellum, measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens or combinations and modifications thereof.

30. The method of claim 27, wherein the composition further comprises antigenic peptides selected from cancer peptides are selected from tumor associated antigens comprising antigens from leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia.

31. The method of claim 27, wherein the composition further comprises antigenic peptides selected from tumor associated antigens are selected from CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67.

32. The method of claim 27, wherein the DC-specific antibody is humanized.

33. The method of claim 27, wherein the composition is administered to the human or animal subject by an oral route, a nasal route, topically or as an injection.

34. The method of claim 33, wherein the injection is selected from the group consisting of subcutaneous, intravenous, intraperitoneal, intramuscular, and intravenous.

35. A method of providing immunostimulation by activation of one or more dendritic cells (DCs) to a human subject for a prophylaxis, a therapy or a combination thereof against one or more viral, bacterial, fungal, parasitic, protozoal, and parasitic diseases, and allergic disorders comprising the steps of:
identifying the human subject in need of immunostimulation for the prophylaxis, the therapy or a combination thereof against the one or more diseases selected from the group selected from influenza, HIV, cancer, and immune disorders;
isolating one or more DCs from the human subject;
activating the isolated DCs with an amount of a composition effective for form activated DCs comprising:
an antigen; and
an adjuvant, wherein the adjuvant comprises an anti-dendritic cell (DC)-specific antibody or fragment thereof conjugated to a Toll-Like Receptor (TLR) agonist; and a pharmaceutically acceptable carrier, wherein the conjugate and agonist are each comprised in an amount such that, in combination with the other, are effective to produce the immune response in a human or animal subject in need of immunostimulation; and
reintroducing the activated DCs into the human subject.

36. The method of claim 35, wherein the DC-specific antibody or fragment is selected from an anti-DCIR, MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, or ASPGR.

37. The method of claim 35, wherein the composition further comprises antigenic peptides selected from human immunodeficiency virus (HIV) antigens and gene products selected from the group consisting of gag, pol, and env genes, the Nef protein, reverse transcriptase, string of HIV peptides (Hipo5), PSA (KLQCVDLHV)-tetramer (SEQ ID NO: 159), a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide (GILGFVFTL) tetramer (SEQ ID NO: 160), and Avian Flu (HA5-1), dockerin domain from C. thermocellum, measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens or combinations and modifications thereof.

38. The method of claim 35, wherein the composition further comprises antigenic peptides selected from cancer peptides are selected from tumor associated antigens comprising antigens from leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia.

39. The method of claim 35, wherein the composition further comprises antigenic peptides selected from tumor associated antigens are selected from CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67.

40. The method of claim 35, wherein the DC-specific antibody is humanized.

41. The method of claim 35, wherein the composition is administered to the human or animal subject by an oral route, a nasal route, topically or as an injection.

42. The method of claim 41, wherein the injection is selected from the group consisting of subcutaneous, intravenous, intraperitoneal, intramuscular, and intravenous.


SEQUENCE LISTING (160 sequences)

SEQ ID NO: 1 - protein, 430 aa, Artificial Sequence
  Description of Artificial Sequence Synthetic polypeptide
MDLDAVRIKVDTVNAKPGDTVNIPVRFSGIPSKGIANADFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASLLTEVETYVLSIIPSGPLKAEIAQRLEDVFAGKNTDLEVLMEWLKTRPILSPLTKGILGFVFTLTVPSERGLQRRRFVQNALNGNGDPNNMDKAVKLYRKLKREITFHGAKEIALSYSAGALASCMGLIYNRMGAVTTEVAFGLVCATCEQIADSQHRSHRQMVTTTNPLIRHENRMVLASTTAKAMEQMAGSSEQAAEAMDIASQARQMVQAMRTIGTHPSSSAGLKDDLLENLQAYQKRMGVQMQRFKLEHHHHHH

SEQ ID NO: 2 - protein, 513 aa, Artificial Sequence
  Description of Artificial Sequence Synthetic polypeptide
QVTLKESGPGILQPSQTLSLTCSFSGFSLSTSGMGLSWIRQPSGKGLEWLAHIYWDDDKRYNPSLKSRLTISKDTSSNQVFLKITIVDTADAATYYCARSSHYYGYGYGGYFDVWGAGTTVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASQTPTNTISVTPTNNSTPTNNSNPKPNPASCQTPTNTISVTPTNNSTPTNNSNPKPCPAS

SEQ ID NO: 3 - DNA, 1409 bp, Artificial Sequence
  Description of Artificial Sequence Synthetic polynucleotide
atgggcaggcttacttcttcattcttgctactgattgtccctgcatatgtcctgtcccaggttactctgaaagagtctggccctgggatattgcagccctcccagaccctcagtctgacctgttctttctctgggttttcactgagcacttctggtatgagtgtaggctggattcgtcagccttcagggaagggtctggagtggctggctcacatttggtggaatgatgataagtactataatccagtcctgaaaagccggctcacaatctccaaggagacctccaacaaccaggtattcctcaagatcgccagtgtggtctctgcagatactgccacatactactgtgctcgattctatggtaactgtcttgactactggggccaaggcaccactctcacagtctcctcggccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctgattaattaa

SEQ ID NO: 4 - DNA, 705 bp, Artificial Sequence
  Description of Artificial Sequence Synthetic polynucleotide
atgatgtcctctgctcagttccttggtctcctgttgctctgttttcaaggtaccagatgtgatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaatattacaattaggagtcccatcaagattcagtggcagtgggtctgaaacagattattctctcaccattagcaacctggagcaagaagatattgccacttacttttgccaacagggtgattcgcttccattcacgttcggctcggggacaaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 5 - DNA, 1403 bp, Artificial Sequence
  Description of Artificial Sequence Synthetic polynucleotide
atgagagcgctgattcttttgtgcctgttcacagcctttcctggtatcctgtctgatgtgcagcttcaggagtcaggacctgacctggtgaaaccttctcagtcactttcactcacctgcactgtcactggctactccatcaccagtggttatagctggcactggatccggcagtttccaggaaacaaactggaatggatgggctacatactcttcagtggtagcactaactacaacccatctctgaaaagtcgaatctctatcactcgagacacatccaagaaccagttcttcctgcagttgaattctgtgactactgaggacacagccacatatttctgtgcaagatctaactatggttcctttgcttcctggggccaagggactctggtcactgtctctgcagccaaaacaacgggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctgattaattaa

SEQ ID NO: 6 - DNA, 708 bp, Artificial Sequence
  Description of Artificial Sequence Synthetic polynucleotide
atggattttcaagtgcagattttcagcttcctgctaatcagtgcctcagtcataatatccagaggacaaattgttctcacccagtctccagcaatcatgtctgcatctccaggggagaaggtcaccatgacctgcagtgccagctcaagtgtaagtcacatgcactggtaccagcagaagtcaggcacttcccccaaaagatggatttatgacacatccagactggcttctggagtccctgctcgcttcagtggcagtgggtctgggacctcttactctctcacaatcagcagcatggaggctgaagatgctgccacttattactgccagcagtggagtagtcacccatggtcgttcggtggaggcaccaaactcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 7 - DNA, 1415 bp, Artificial Sequence
  Description of Artificial Sequence Synthetic polynucleotide
atggcttgggtgtggaccttgctattcctgatggcagctgcccaaagtgcccaagcacagatccagttggtgcagtctggacctgagctgaagaagcctggagagacagtcaagatctcctgcaaggcttctgggtataccttcacaaactatggaatgaactgggtgaagcaggttccaggaaaaggtttaaggtggatgggctggatggacaccttcactggagagccaacatatgctgatgacttcaagggacggtttgccttctctttggaaacctctgccagcactgcctatttgcagatcaacagcctcaaaaatgaggacacggctacttatttctgtgcaagaggggggattttacgactcaactactttgactactggggccaaggcaccactctcacagtctcctcagccaaaacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctgattaattaa

SEQ ID NO: 8 - DNA, 705 bp, Artificial Sequence
  Description of Artificial Sequence Synthetic polynucleotide
atgaagtttccttctcaacttctgctcttactgctgtttggaatcccaggcatgatatgtgacatccagatgacacaatcttcatcctccttttctgtatctctaggagacagagtcaccattacttgcaaggcaagtgaggacatatataatcggttaggctggtatcagcagaaaccaggaaatgctcctaggctcttaatatctggtgcaaccagtttggaaactggggttccttcaagattcagtggcagtggatctggaaaggattacgctctcagcattaccagtcttcagactgaagatcttgctacttattactgtcaacagtgttggacttctccgtacacgttcggaggggggaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 9 - DNA, 1398 bp, Artificial Sequence
  Description of Artificial Sequence Synthetic polynucleotide
atgggatggagctggatctttctctttctcttgtcaggaactggaggtgtcctctctgaggtccagctgcaacagtctggacctgagctggtgaagcctggggcttcagtgaagatgtcctgcaaggcttctggatacaccttcactgactactacatgaagtgggtgaagcagagccatggaaagagccttgagtggattggagatattaatcctaactatggtgatactttctacaaccagaagttcgagggcaaggccacattgactgtagacaaatcctccaggacagcctacatgcagctcaacagcctgacatctgaggactctgcagtctattattgtggaagaggggactatggatacttcgatgtctggggcgcagggaccacggtcaccgtctcctcagccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 10 - DNA, 705 bp, Artificial Sequence
  Description of Artificial Sequence Synthetic polynucleotide
atggagacacattctcaggtctttgtatacatgttgctgtggttgtctggtgttgaaggagacattgtgatgacccagtctcacaaattcatgtccacatcagtaggagacagggtcagcatcacctgcaaggccagtcaggatgtgggtactgctgtagcctggtatcaacagaaaccagggcaatctcctaaactactgatttactgggcatccacccggcacactggagtccctgatcgcttcacaggcagtggatctgggacagatttcactctcaccattaacaatgtgcagtctgaagacttggcagattatttctgtcagcaatatagcagcaatccgtacatgttcggaggggggaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 11 - DNA, 1413 bp, Artificial Sequence
  Description of Artificial Sequence Synthetic polynucleotide
atgggatggagctggatctttctctttctcctgtcaggaactgcaggtgtcctctctgaggtccagctgcaacagtctggacctgagttggtgaagcctggggcttcagtgaagatatcctgcaagacttctggatacacattcactgaatacaccatgcactgggtgaggcagagccatggaaagagccttgagtggattggaggtattaatcctatcaatggtggtcctacctacaaccagaagttcaagggcaaggccacattgactgttgacaagtcctccagcacagcctacatggagctccgcagcctgacatctgaggactctgcagtctattactgtgcaagatgggactatggtagtcgagatgttatggactactggggtcaaggaacctcagtcaccgtctcctcagccaaaacgaagggcccatccgtcttccccctggcgccctgctccaggagcacctccgagagcacagccgccctgggctgcctggtcaaggactacttccccgaaccggtaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 12 - DNA, 705 bp, Artificial Sequence
  Description of Artificial Sequence Synthetic polynucleotide
atggaatcacagactctggtcttcatatccatactgctctggttatatggtgctgatgggaacattgtaatgactcaatctcccaaatccatgtccatgtcagtaggggagagggtcaccttgagctgcaaggccagtgagaatgtgggaacttatgtatcctggtatcaacagagaccagaacagtctccaaaactgctgatatacggggcatccaaccggtacactggggtccccgatcgcttcacaggcagtggatctgcaacagatttcactctgaccatcagcagtgtgcaggctgaggaccttgcagattatcactgtggacagacttacagctatatattcacgttcggctcggggacaaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 13 - DNA, 1404 bp, Artificial Sequence
  Description of Artificial Sequence Synthetic polynucleotide
atgggatggagccggatctttctcttcctcctgtcaataactgcaggtgtccattgccaggtccaggtgcagcagtcgggacctgagttggtgaagcctggggcctcagtgaagatttcctgcaaagcctctggcgacgcattcagtagttcttggatgaactgggtgaagcagaggcctggacagggtcttgagtggattggacggatttatcttggagatggagatattaattacaatgggaagttcaagggcagggccacactgactgcagacaaatcctccagcacagcctacatgcagctcagcagcctgacctctgtggactctgcggtctatttctgcgcgaggcagctcgggctatggtatgttatggactactggggtcaaggaacctcagtcaccgtctcctcagccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 14 - DNA, 705 bp, Artificial Sequence
  Description of Artificial Sequence Synthetic polynucleotide
atgagtgtgcccactcaggtcctggggttgctgctgctgtggcttacaggtgccagatgtgacatccagatggctcagtctccagcctccctatctgcatctgtgggagaaactgtcaccatcacatgtcgagcaagtgagaatatttacagttatttagcatggtatcagcagaaacagggaaaatctcctcagctcctggtctataatgcaaaaaccttagcagaaggtgtgccatcaaggttcagtggcagtggatcaggcacacagttttctctgaagatcaacagcctgcagcctgaagattttgggagttattactgtcaacatcattatggttttccgtggacgttcggtggaggcaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 15 - DNA, 1395 bp, Artificial Sequence
  Description of Artificial Sequence Synthetic polynucleotide
atgaacttcgggctcagcttgattttccttgtcctcattttaaaaggtgtccagtgtgaggtgcagctggtggagtctgggggagacttagtgaagcctggagggtccctgaaactctcctgtgcagcctctggattcactttcagtagctatggcatgtcttgggttcgccagactccagacaagaggctggagtgggtcgcagtcattagtagtggtggaagttccaccttctatccagacagtgtgaaggggcgattcaccatctccagagacaatgccaagaacaccctgtacctgcaaatgagcagtctgaagtctgaggacacagccgtgtattactgttcaagaggaggttactactttgactactggggccaaggcaccactctcacagtctccgcagccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 16 - DNA, 705 bp, Artificial Sequence
  Description of Artificial Sequence Synthetic polynucleotide
atgaggttccaggttcaggttctggggctccttctgctctggatatcaggtgcccagtgtgatgtccagataacccagtctccatcttatcttgctgcatctcctggagaaaccattactattaattgcagggcaagcaagaccattagcaaatatttagcctggtatcaagagaaacctgagaaaactgataagcttcttatctactctggatccactttgcaatctggaattccatcaaggttcagtggcagtggatctggtacagatttcactctcaccatcagtggcctggagcctgaagattttgcaatgtattactgtcaacagcataatgaatacccgtggacgttcggtggaggcaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 17 - DNA, 1389 bp, Artificial Sequence
  Description of Artificial Sequence Synthetic polynucleotide
atgggatggagctggatctttctctttctcctgtcaggaactgcaggtgtcctctctgaggtccagctgcaacagtctggacctgagctggtgaagcctggggcttcagtgaagatatcctgcaaggcttctggttactcattcactggctactacatgcactgggtgaagcaaagccatgtaaagagccttgagtggattggacgtattaatccttacaatggtgctactagctacaaccagaatttcaaggacaaggccagcttgactgtagataagtcctccagcacagcctacatggagctccacagcctgacatctgaggactctgcagtctattactgtgcaagagaggactacgtctactggggccaaggcaccactctcacagtctcctcagccaaaacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 18 - DNA, 717 bp, Artificial Sequence
  Description of Artificial Sequence Synthetic polynucleotide
atgaagttgcctgttaggctgttggtgctgatgttctggattcctgcttccagcagtgatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcgcactcaagatcagtagagtggaggctgaggatctgggagtttatttctgctctcaaagtacacatgttccgtggacgttcggtggaggcaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 19 - DNA, 1410 bp, Artificial Sequence
  Description of Artificial Sequence Synthetic polynucleotide
atggaatggagttggatatttctctttcttctgtcaggaactgcaggtgtccactctgaggtccagctgcagcagtctggacctgagctggtaaagcctggggcttcagtgaagatgtcctgcaaggcttctggatacacattcactgactatgttttgcactgggtgaaacagaagcctgggcagggccttgagtggattggatatattaatccttacaatgatggtactaagtacaatgagaagttcaaaggcaaggccacactgacttcagacaaatcctccagcacagcctacatggagctcagcagcctgacctctgaggactctgcggtctattactgtgcaaggggctatccggcctactctgggtatgctatggactactggggtcaaggaacctcagtcaccgtctcctcagccaaaacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 20 - DNA, 705 bp, Artificial Sequence
  Description of Artificial Sequence Synthetic polynucleotide
atgatgtcctctgctcagttccttggtctcctgttgctctgttttcaaggtaccagatgtgatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccatcatggtaatacgcttccgtggacgttcggtggaggcaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 21 - DNA, 1404 bp, Artificial Sequence
  Description of Artificial Sequence Synthetic polynucleotide
atgaacttggggctcagcttgattttccttgtccttgttttaaaaggtgtccagtgtgaagtgaagctggtggagtctgggggaggcttagtgcagcctggagggtccctgaaactctcctgtgcaacctctggattcactttcagtgactattacatgtattgggttcgccagactccagagaagaggctggagtgggtcgcatacattaattctggtggtggtagcacctattatccagacactgtaaagggccgattcaccatctccagagacaatgccaagaacaccctgtacctgcaaatgagccggctgaagtctgaggacacagccatgtattactgtgcaagacgggggttaccgttccatgctatggactattggggtcaaggaacctcagtcaccgtctcctcagccaaaacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 22 - DNA, 705 bp, Artificial Sequence
  Description of Artificial Sequence Synthetic polynucleotide
atgatgtcctctgctcagttccttggtctcctgttgctctgttttcaaggtaccagatgtgatatccagatgacacagactacatcctccctgtctgcctctctaggagacagagtcaccatcagttgcagtgcaagtcagggcattagcaattatttaaactggtatcagcagaaaccagatggaactgttaaactcctgatctattacacatcaattttacactcaggagtcccatcaaggttcagtggcagtgggtctgggacagattattctctcaccatcggcaacctggaacctgaagatattgccacttactattgtcagcagtttaataagcttcctccgacgttcggtggaggcaccaaactcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 23 - DNA, 1405 bp, Artificial Sequence
  Description of Artificial Sequence Synthetic polynucleotide
atggattggctgtggaacttgctattcctgatggcagctgcccaaagtgcccaagcacagatccagttggtgcagtctggacctgagctgaagaagcctggagagacagtcaagatctcctgcaaggcttctgggtattccttcacaaactatggaatgaactgggtgaaacaggctccaggaaagggtttaaagtggatgggctggataaacacctacactggagagtcaacatatgctgatgacttcaagggacggtttgccttctctttggaaacctctgccagcactgcctatttgcagatcagtaacctcaaaaatgaggacatggctacatatttctgtgctagaggggactttaggtactactattttgactactggggccaaggcaccactctcacaggctcctcagccaaaacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctgat

SEQ ID NO: 24 - DNA, 702 bp, Artificial Sequence
  Description of Artificial Sequence Synthetic polynucleotide
atgagtgtgctcactcaggtcctggcgttgctgctgctgtggcttacaggtgccagatgtgacatccagatgactcagtctccagcctccctatctgcatctgtgggagaaactgtcaccatcacgtgtcgagcaagtgggaatattcacaattatttagcatggtatcagcagaaacagggaaaatctcctcagctcctggtctataatgcaaaaaccttggcagatggtgtgccatcaaggttcagtggcagtggatcaggaacacaatattctctcaagatcaacaccctgcagcctgaagattttgggagttattactgtcaacatttttgggattcttggacgttcggtggaggcaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 25 - DNA, 1398 bp, Artificial Sequence
  Description of Artificial Sequence Synthetic polynucleotide
atggaatggacctgggtctttctcttcctcctgtcagtaactgcaggtgtccactcccaggttcagctgcagcagtctggagctgagctgatgaagcctggggcctcagtgaagatatcctgcaaggctactggctacacattcagtagctactggatagagtgggtaaagcagaggcctggacatggccttgagtggattggagagattttacctggaagtggtaggactaacgacaatgagaagttcaagggcaaggccacattcactgcagatacatcctccaagaaagcctacatgcaactcagcagcctgacatctgaggactctgccgtctattattgtgcaagaaggggtggttactcctttgcttactggggccaagggactctggtcactgtctctgcagccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 26 - DNA, 1422 bp, Artificial Sequence
  Description of Artificial Sequence Synthetic polynucleotide
atgaccatgttctcactagctcttctcctcagtcttcttctcctctgtgtctctgattctagggcagaaacaactgtgacccagtctatgaccatgttctcactagctcttctcctcagtcttcttctcctctgtgtctctgattctagggcagaaacaactgtgacccagtctccagcatccctgtccatggctataggggaaaaagtcaccatcagatgcgtaaccagcactgatattgatgatgatgtgaactggtaccagcagaagccaggggaacctcctaaactccttatttcagaaggcaatactcttcgtcctggagtcccatcccgattctccagcagtggctatggtacagattttgtttttacaattgagaacatgctctcagaagatgttgcagattactactgtttgcaaagtggtaacttgccgtacacgttcggaggggggaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttagccagcatccctgtccatggctataggggaaaaagtcaccatcagatgcgtaaccagcactgatattgatgatgatgtgaactggtaccagcagaagccaggggaacctcctaaactccttatttcagaaggcaatactcttcgtcctggagtcccatcccgattctccagcagtggctatggtacagattttgtttttacaattgagaacatgctctcagaagatgttgcagattactactgtttgcaaagtggtaacttgccgtacacgttcggaggggggaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 27 - DNA, 1404 bp, Artificial Sequence
  Description of Artificial Sequence Synthetic polynucleotide
atggcttgggtgtggaccttgctattcctgatggcagctgcccaaagtgcccaagcacagatccagttggtgcagtctggacctgagctgaagaagcctggagagacagtcaagatctcctgcaaggcttctgggtataccttcacaaactatggaatgaactgggtgaagcaggctccaggaaagggtttaaagtgggtgggctggataaacaccttcactggagagccaacatatgttgatgacttcaagggacggtttgccttctctttggaaacctctgccagcactgcctatttgcagatcaacaacctcaaaaatgaggacacggctacatatttctgtgcaagagggaattttaggtactactactttgactactggggccaaggcaccactctcacagtctcctcagccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 28 - DNA, 705 bp, Artificial Sequence
  Description of Artificial Sequence Synthetic polynucleotide
atgagtgtgctcactcaggtcctggcgttgctgctgctgtggcttacaggtgccagatgtgacatccagatgactcagtccccagcctccctatctgcatctgtgggagaaactgtcaccatcacatgtcgaacaagtgggaatattcgcaattatttagcatggtatcagcagaaacagggaaaatctcctcaactcctggtctataatgcaaaaaccttagcagatggtgtgccatcaaggttcggtggcagtggatcaggaacacaatattctctcaagatcaacagcctgcagcctgaagattttgggaattattactgtcaacatttttggagtagtccgtacacgttcggaggggggaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 29 - DNA, 2811 bp, Artificial Sequence
  Description of Artificial Sequence Synthetic polynucleotide
atgatgggatggagctatatcatcctctttttggtagcaacagctacagatgtccactcccaggtccaactgcagcagcctggggctgaactggtgaagcctggggcttcagtgaagctgtcctgcaaggcttctggctacaccttcaccagctactggatgcactgggtgaagcagaggcctggagaaggccttgagtggattggagagattaatcctagctacggtcgtactgactacaatgagaagttcaagaacaaggccacactgactgtagccaaatcctccagcacagcctacatgcaactcagcagcctgacatctgaggactctgcggtctattactgtgcaagaggagattactacggtagtagctcgtttgcttactggggccaagggactctggtcactgtctctgcagccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagcggatggagctatatcatcctctttttggtagcaacagctacagatgtccactcccaggtccaactgcagcagcctggggctgaactggtgaagcctggggcttcagtgaagctgtcctgcaaggcttctggctacaccttcaccagctactggatgcactgggtgaagcagaggcctggagaaggccttgagtggattggagagattaatcctagctacggtcgtactgactacaatgggaagttcaagaacaaggccacactgactgtagccaaatcctccagcacagcctacatgcaactcagcagcctgacatctgaggactctgcggtctattactgtgcaagaggagattactacggtagtagctcgtttgcttactggggccaagggactctggtcactgtctctgcagccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 30 - DNA, 708 bp, Artificial Sequence
  Description of Artificial Sequence Synthetic polynucleotide
atggattttcaagtgcagattttcagcttcctgctaatgagtgcctcagtcataatgtccaggggacaaattgttctcacccagtctccagcactcatgtctgcatctccaggggagaaggtcaccatgacctgcagtgccagctcaaatataagttacatgtactggtaccagcagaagccaagatcctcccccaaaccctggatttatctcacatccaacctggcttctggagtccctgctcgcttcagtggcagtgggtctgggacctcttactctctcacaaccagcagcatggaggctgaagatgctgccacttattgctgccagcagtggagtagtaacccacccacgttcggtgctgggaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 31 - DNA, 708 bp, Artificial Sequence
  Description of Artificial Sequence Synthetic polynucleotide
atggattttcgagtgcagattttcagcttcctgctaatgagtgcctcagtcataatgtccaggggacaaattgttctcacccagtctccagcactcatgtctgcatctccaggggagaaggtcaccatgacctgcagtgccagctcaaatataagttacatgtactggtaccagcagaagccaagatcctcccccaaaccctggatttatctcacatccaacctggcttctggagtccctgctcgcttcagtggcagtgggtctgggacctcttactctctcacaatcagcagcatggaggctgaagatgctgccacttattactgccagcagtggagtagtaacccacccacgttcggtgctgggaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 32 - DNA, 717 bp, Artificial Sequence
  Description of Artificial Sequence Synthetic polynucleotide
atggagacagacacactcctgctatgggtgctgctgctctgggttccaggttccacaggtgacattgtgctgatccaatctccagcttctttggctgtgtctctagggcagagggccaccatatcctgcagagccagtgaaagtgttgatagttatgtcaatagttttatgcactggtaccagcagaaaccaggacagccacccaaactcctcatctatcgtgtatccaacctagaatctgggatccctgccaggttcagtggcagtgggtctaggacagacttcaccctcaccattaatcctgtggaggctgatgatgttgcaacctattactgtcagcaaagtaatgaggatccattcacgttcggctcggggacaaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 33 - DNA, 1398 bp, Artificial Sequence
  Description of Artificial Sequence Synthetic polynucleotide
atggaatgtaactggatacttccttttattctgtcggtaatttcaggggtctactcagaggttcagctccagcagtctgggactgtgctggcaaggcctggggcttccgtgaatatgtcctgtaaggctgctggctacagctttaccagttactgggtgtactgggtcaaacagaggcctggacagggtctggaatggattggtgctatttaccctaaaaatagtagaactagctacaaccagaagttccaggacaaggccacactgactgcagtcacatccgccagcactgcctacatggagctcagcagcctgacaaatgaggactctgcggtctattactgtacaagacctcactatgattcgtttggttactggggccaagggactctggtcactgtctctgcagccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 34 - DNA, 717 bp, Artificial Sequence
  Description of Artificial Sequence Synthetic polynucleotide
atggagacagacacactcctgctatgggtgctgctgctctgggttccaggttccacaggtgacattgtgctgacccaatctccagcttctttggctgtgtctctagggcagagggccaccatatcctgcagagccagtgaaagtgtagatagttatggcattagttttatgcactggtaccagcagaaaccaggacagccacccaaactcctcatctatcgtgcatccaaccaagaatctgggatccctgccaggttcagtggcagtgggtctaggacagacttcaccctcaccattaatcctgtggaggctgatgatgttgcaacctattactgtcagcaaagtaatgaggatccgctcacgttcggtgctgggaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 35 - DNA, 1422 bp, Artificial Sequence
  Description of Artificial Sequence Synthetic polynucleotide
atgaacaggcttacttcctcattgctgctgctgattgtccctgcatatgtcctgtcccaggttactctgaaagagtctggccctgggatattgcagccctcccagaccctcagtctgacttgttctttctctgggttttcactgagcacttctggtatgggtgtgagctggattcgtcagccttcaggaaagggtctggagtggctggcacacatttactgggatgatgacaagcgctataatccatccctgaagagccggctcacaatctttaaggatccctccagcaaccaggtattcctcaggatcaccagtgtggacactgcagatactgccacatactactgtgctcgaaactcccattactacggtagtacttacgggggatacttcgatgtctggggcgcagggaccacggtcaccgtctcctcagccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 36 - DNA, 717 bp, Artificial Sequence
  Description of Artificial Sequence Synthetic polynucleotide
atggagacagacacactcctgctatgggtgctgctgctcggggttccaggttccacaggtaacattgtgctgacccagtctccaacttctttcactgtgtctcttgggcagagggccaccatatcctgcagagccagtgaaagtgttcatagttatggcaatagttttatgcactggtaccagcagaaaccagggcagccacccaaactcctcatctatcttgcatccaacgtagaatctggggtccctgccaggttcagtggtagtgggtccaggacagacttcaccctcaccattgatcctgtggaggctgatgatgctgcaacctattactgtcagcaaaatagtgaggatccgtggacgttcggtggaggcaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 37 - DNA, 1398 bp, Artificial Sequence
  Description of Artificial Sequence Synthetic polynucleotide
atggaatggacctgggtctttctcttcctcctgtcagtaactgcaggtgtccactcccaggttcagctgcagcagtctggaactgagctgatgaagcctggggcctcagtgaagatatcctgcaaggctactggctacacattcagtacctactggatagagtgggtaaagcagaggcctggacatggccttgagtggattggagagattttacctggaagtggtaggactaacgacaatgagaagttcaagggcaaggccacaatcactgcagatacatcctccaagaaagcctacatgcaactcagcagcctgacatctgaggactctgccgtctattactgtgcaagaaggggtggttactcctttgctttctggggccaagggactctggtctctgtctctgcagccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 38 - DNA, 711 bp, Artificial Sequence
  Description of Artificial Sequence Synthetic polynucleotide
atgaccatgttctcactagctcttctcctcagtcttcttctcctctgtgtctctgattctagggcagaaacaactgtgacccagtctccagcatccctgtccatggctataggagaaaaagtcaccatcagatgcgtaaccagcactgatattgatgatgatgtgaactggtaccagcagaagccaggggaacctcctaagctccttatttcagaaggcaatactcttcgtgctggagtcccatcccgattctccagcagtggctatggtacagattttgtttttacaattgagaacatgctctcagaagatgttgcagattactactgtttgcaaagtggtaacttgccgtacacgttcggaggggggaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 39 - DNA, 1422 bp, Artificial Sequence
  Description of Artificial Sequence Synthetic polynucleotide
atgaacaggcttacttcctcattgctgctgctgattgtccctgcatatgtcctgtcccaggttactctgaaagagtctggccctgggatattgcagccctcccagaccctcagtctgacttgttctttctctgggttttcactgagcacttctggtatgggtctgagctggattcgtcagccttcaggaaagggtctggagtggctggcacacatttactgggatgatgacaagcgctataacccatccctgaagagccggctcacaatctccaaggatacctccagcaaccaggttttcctcaagatcaccattgtggacactgcagatgctgccacatactactgtgctcgaagctcccattactacggttatggctacgggggatacttcgatgtctggggcgcagggaccacggtcaccgtctcctcagccaaaacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 40 - DNA, 823 bp, Artificial Sequence
  Description of Artificial Sequence Synthetic polynucleotide
atggagacagacacactcctgctatgggtgctgctgctctgggttccaggttccacaggtaacattgtgctgacccaatctccagcttctttggctgtgtctctagggcagagggccaccatatcctgcagagccagtgaaagtattcatagttatggcaatagttttctgcactggtaccagcagaaaccaggacagccacccaaactcctcatctatcttgcatccaacctagaatctggggtccctgccaggttcagcggcagtgggtctaggacagacttcaccctcaccattgatcctgtggaggctgatgatgctgcaacctattactgtcagcaaaataatgaggatccgtggacgttcggtggaggcaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttaggcggccgcactagcgcgggccgcattcgaagagctcggtacccggggatcctctagagtcgacctgcaggcatgcaagctggccgcgactctagatcataatcagc

SEQ ID NO: 41 - DNA, 1404 bp, Artificial Sequence
  Description of Artificial Sequence Synthetic polynucleotide
atgaaatgcagctgggtcatcttcttcctgatggcagtggttacaggggtcaattcagaggttcagctgcagcagtctggggctgagcttgtgaggccaggggccttagtcaagttgtcctgcaaagcttctggcttcaacattaatgactactatatccactgggtgaagcagcggcctgaacagggcctggagcggattggatggattgatcctgacaatggtaatactatatatgacccgaagttccagggcaaggccagtataacagcagacacatcccccaacacagcctacctgcagctcagcagcctgacatctgaggacactgccgtctattactgtgctagaacccgatctcctatggttacgacggggtttgtttactggggccaagggactgtggtcactgtctctgcagccaaaacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacraagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 42 - DNA, 1425 bp, Artificial Sequence
  Description of Artificial Sequence Synthetic polynucleotide
atggaaaggcactggatctttctcttcctgttttcagtaactgcaggtgtccactcccaggtccagcttcagcagtctggggctgagctggcaaaacctggggcctcagtgaagatgtcctgcaaggcttctggctacacctttactacctactggatgcactgggtaaaacagaggcctggacagggtctggaatggattggatacattaatcctatcactggttatactgagtacaatcagaagttcaaggacaaggccaccttgactgcagacaaatcctccagcacagcctacatgcaactgagcagcctgacatctgaggactctgcagtctattactgtgcaagagagggtttaagtgctatggactattggggtcagggaacctcagtcaccgtcacctcagccaaaacaacagccccatcggtctatccactggcccctgtgtgtggagatacaactggctcctcggtaactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaagctagctga

SEQ ID NO: 43 - DNA, 717 bp, Artificial Sequence
  Description of Artificial Sequence Synthetic polynucleotide
atgggcatcaagatggagtcacggattcaggcatttgtattcgtgtttctctggttgtctggtgttggcggagacattgtgatgacccagtctcacaaattcatgtccacatcagtaggagacagggtcagcgtcacctgcaaggccagtcaggatgtgacttctgctgtagcctggtatcaacaaaaaccagggcaatctcctaaactactgatttactgggcatccacccggcacactggagtccctgatcgcttcacaggcagtggatctgggacagattatactctcaccatcagcagtgggcaggctgaagacctggcactttattactgtcaccaatattatagcgctcctcggacgttcggtggaggcaccaagctggaagtcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccatcgtcaagagcttcaataggaatgagtgttag

SEQ ID NO: 44 - DNA, 1398 bp, Artificial Sequence
  Description of Artificial Sequence Synthetic polynucleotide
atggaaaggcactggatctttctcttcctgttttcagtaactgcaggtgtccactcccaggtccagcttcagcagtctggggctgagctggcaaaacctggggcctcagtgaagatgtcctgcaaggcttctggctacacctttactacctactggatgcactgggtaaaacagaggcctggacagggtctggaatggattggatacattaatcctatcactggttatactgagtacaatcagaagttcaaggacaaggccaccttgactgcagacaaatcctccagcacagcctacatgcaactgagcagcctgacatctgaggactctgcagtctattactgtgcaagagagggtttaagtgctatggactattggggtcagggaacctcagtcaccgtcacctcagccaaaacaacgggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 45 - DNA, 717 bp, Artificial Sequence
  Description of Artificial Sequence Synthetic polynucleotide
atgggcatcaagatggagtcacagattcaggcatttgtattcgtgtttctctggttgtctggtgttggcggagacattgtgatgacccagtctcacaaattcatgtccacatcagtaggagacagggtcagcgtcacctgcaaggccagtcaggatgtgacttctgctgtagcctggtatcaacaaaaaccagggcaatctcctaaactactgatttactgggcatccacccggcacactggagtccctgatcgcttcacaggcagtggatctgggacagattatactctcaccatcagcagtgggcaggctgaagacctggcactttattactgtcaccaatattatagcgctcctcggacgttcggtggaggcaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 46 - DNA, 1401 bp, Artificial Sequence
  Description of Artificial Sequence Synthetic polynucleotide
atggctgtcctggcactactcctctgcctggtggctttcccaacttgtaccctgtcccaggtgcaactgaaggagtcaggacctggcctggtggcgccctcacagagcctgtccattacctgctctgtctctgggttctcattaagcaactatgatataagctggattcgccagccaccaggaaagggtctggagtggcttggagtaatgtggactggtggaggcgcaaattataattcagctttcatgtccagactgagcatcaacaaggacaactccaagagccaagtttttttaaaaatgaacaatctgcaaactgatgacacagccatttattactgtgtcagagatgcggtgaggtactggaacttcgatgtctggggcgcagggaccacggtcaccgtctcctcagccaaaacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 47 - DNA, 708 bp, Artificial Sequence
  Description of Artificial Sequence Synthetic polynucleotide
atggattttcaagcgcagattttcagcttcctgctaatcagtgcttcagtcataatgtccagaggacaaattgttctctcccagtcaccagcaatcctgtctgcatctccaggggagaaggtcacaatgacttgcagggccagctcaagtgtaagttacatacactggtaccagcagaagccaggatcctcccccaaaccctggatttatgccacatcccacctggcttctggagtccctgctcgcttcagtggcagtgggtctgggacctcttactctctcacaatcagcagagtggaggctgaagatactgccacttattactgccagcagtggagtagtaacccattcacgttcggctcggggacaaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 48 - DNA, 1410 bp, Artificial Sequence
  Description of Artificial Sequence Synthetic polynucleotide
atggaaaggcactggatctttctactcctgttgtcagtaactgcaggtgtccactcccaggtccagctgcagcagtctggggctgaactggcaagacctggggcctcagtgaagatgtcctgcaaggcttctggctacacctttactacctacactatgcactgggtaaaacagaggcctggacagggtctggaatggattggatacattaatcctagcagtggttatactaattacaatcagaagttcaaggacaaggccacattgactgcagacaaatcctccagcacagcctccatgcaactgagcagcctgacatctgaggactctgcagtctattactgtgcaagagagagggcggtattagtcccctatgctatggactactggggtcaaggaacctcagtcaccgtctcctcagccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 49 - DNA, 708 bp, Artificial Sequence
  Description of Artificial Sequence Synthetic polynucleotide
atgcattttcaagtgcagattttcagcttcctgctaatcagtgcctcagtcataatgtccagaggacaaattgttctcacccagtctccagcagtcatgtctgcatctccaggggagaaggtcaccataacctgcactgccagctcaagtttaagttacatgcactggttccagcagaagccaggcacttctcccaaactctggctttatagcacatccatcctggcttctggagtccctactcgcttcagtggcagtggatctgggacctcttactctctcacaatcagccgaatggaggctgaagatgctgccacttattactgccagcaaaggagtagttccccattcacgttcggctcggggacaaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 50 - DNA, 1404 bp, Artificial Sequence
  Description of Artificial Sequence Synthetic polynucleotide
atgggatggacctggatctttattttaatcctgtcagttactacaggtgtccactctgaggtccagctgcagcagtctggacctgagctggagaagcctggcgcttcagtgaagatatcctgcaaggcttctggttactccttcactggctacaacatgaactgggtgaaacagagcaatggaaagagccttgagtggattggaaatattgatccttactatggtgatactaactacaaccagaagttcaagggcaaggccacattgactgtagacaaatcctccagcacagcctacatgcacctcaagagcctgacatctgaggactctgcagtctattactgtgcaagaccctacggtagtgaggcctactttgcttactggggccaagggactctggtcactgtctctgcagccaaaacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 51 - DNA, 705 bp, Artificial Sequence
  Description of Artificial Sequence Synthetic polynucleotide
atggtgtccacttctcagctccttggacttttgcttttctggacttcagcctccagatgtgacattgtgatgactcagtctccagccaccctgtctgtgactccaggagatagagtctctctttcctgcagggccagccagagtattagcgactacttacactggtatcaacaaaaatcacatgagtctccaaggcttctcatcaaatatgctgcccaatccatctctgggatcccctccaggttcagtggcagtggatcagggtcagatttcactctcagtatcaacggtgtggaacctgaagatgttggagtgtattactgtcaaaatggtcacagctttccgtacacgttcggaggggggaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 52 - DNA, 1395 bp, Artificial Sequence
  Description of Artificial Sequence Synthetic polynucleotide
atggaatggaggatctttctcttcatcctgtcaggaactgcaggtgtccactcccaggttcagctgcggcagtctggacctgagctggtgaagcctggggcttcagtgaagatgtcctgcaaggcttctggatacacatttactgactatgttataagttgggtgaagcagagaactggacagggccttgagtggattggagatatttatcctggaagtggttattctttctacaatgagaacttcaagggcaaggccacactgactgcagacaaatcctccaccacagcctacatgcagctcagcagcctgacatctgaggactctgcggtctatttctgtgcaacctactataactacccttttgcttactggggccaagggactctggtcactgtctctgcagccaaaacaacgggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 53 - DNA, 717 bp, Artificial Sequence
  Description of Artificial Sequence Synthetic polynucleotide
atgaagttgcctgttaggctgttggtgctgatgttctggattcctgcttccagcagtgatgttgtgatgacccaaactccactctccctgcctgtccgtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacaaatttcacactcaagatcagcagagtggaggctgaggatctgggactttatttctgctctcaaagtacacatgttccgtacacgttcggaggggggaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 54 - DNA, 1410 bp, Artificial Sequence
  Description of Artificial Sequence Synthetic polynucleotide
atgacattgaacatgctgttggggctgaggtgggttttctttgttgttttttatcaaggtgtgcattgtgaggtgcagcttgttgagtctggtggaggattggtgcagcctaaagggtcattgaaactctcatgtgcagcctctggattaaccttcaatatctacgccatgaactgggtccgccaggctccaggaaagggtttggaatgggttgctcgcataagaaataaaagtaataattatgcaacatattatgccgattcagtgaaagacaggttcaccatctccagagatgattcacaaagcttgctctatctgcaaatgaacaacttgaaaactgaggacacagccatgtattactgtgtgggacgggactggtttgattactggggccaagggactctggtcactgtctctgcagccaaaacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 55 - DNA, 708 bp, Artificial Sequence
  Description of Artificial Sequence Synthetic polynucleotide
atggcctggatttcacttatactctctctcctggctctcagctcaggggccatttcccaggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagtttcaggtgttcctgccagattctcaggctccctgattggagacaaggctgccctcaccatcacaggggcacagactgaggatgaggcaatatatttctgtgctctatggtacagcaaccattgggtgttcggtggaggaaccaaactcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 56 - DNA, 1386 bp, Artificial Sequence
  Description of Artificial Sequence Synthetic polynucleotide
atggaatggacctgggtctttctcttcctcctgtcagtaactgcaggtgtccactcccaggttcagctgcagcagtctggagctgagctgatgaagcctggggcctcagtgaagatatcctgcaaggctactggctacacattcggtagctactggatagagtgggtaaagcagaggcctggacatggccttgagtggattggagagattttacctggaagtggtaatactaactacaatgagaacttcaagggcaaggccacattcactgcagatacatcctccaacacagcctacatgcaactcaccagtctgacatctgaggactctgccgtctattactgtgctagggcggggatttattggggccaagggactctggtcactgtctctgcagccaaaacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 57 - DNA, 717 bp, Artificial Sequence
  Description of Artificial Sequence Synthetic polynucleotide
atggagaaagacacactcctgctatgggtcctgcttctctgggttccaggttccacaggtgacattgtgctgacccaatctccagcttttttggctgtgtctctagggcagagggccaccatctcctgcagagccagcgaaagtgttgataattatggcattagttttatgaactggttccaacagaaaccaggacagccacccaaactcctcatctatgttgcatccaagcaaggatccggggtccctgccaggtttagtggcagtgggtctgggacagacttcagcctcaacatccatcctatggaggaggatgatactgcaatgtatttctgtcagcaaagtaaggaggttcctcggacgttcggtggaggcaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 58 - DNA, 1392 bp, Artificial Sequence
  Description of Artificial Sequence Synthetic polynucleotide
atggaatgtaactggatacttccttttattctgtcggtaacttcaggggtctactcagaggttcagctccagcagtctgggactgtgctggcaaggcctggggcttcagtgaagatgtcctgcaaggcttctggctacacctttaccagctactggatgcactgggtaaaacagaggcctggacagggtctggaatggattggcgctatttatcctggaaatagtgatactacctacaaccagaagttcaagggcaaggccaaactgactgcagtcacatccaccagcactgcctacatggagctcagcagcctgacaaatgaggactctgcggtctattactgtacacctacttactactttgactactggggccaaggcacctctctcacagtctcctcagccaaaacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 59 - DNA, 717 bp, Artificial Sequence
  Description of Artificial Sequence Synthetic polynucleotide
atgagtcctgcccaattcctgtttctgttagtgctctggattcgggaaaccaacggtgatgttgtgatgacccagactccactcactttgtcggttaccattggacaaccagcctccatctcttgcaagtcaagtcagagcctcttagatagtgatggaaagacatatttgaattggttcttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttccgtggacgttcggtggaggcaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 60 - DNA, 1415 bp, Artificial Sequence
  Description of Artificial Sequence Synthetic polynucleotide
atgggagggatctggatctttctcttcctcctgtcaggaactgcaggtgcccactctgagatccagctgcagcagactggacctgagctggtgaagcctggggcttcagtgaagatatcctgcaaggcttctggttatccattcactgactacatcatggtctgggtgaagcagagccatggaaagagccttgagtggattggaaatattagtccttactatggtactactaactacaatctgaagttcaagggcaaggccacattgactgtagacaaatcttccagcacagcctacatgcagctcaacagtctgacatctgaggactctgcagtctattactgtgcaagatcccctaactgggacggggcctggtttgctcactggggccaaggggctctggtcactgtctctgcagccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctgattaattaa

SEQ ID NO: 61 - DNA, 717 bp, Artificial Sequence
  Description of Artificial Sequence Synthetic polynucleotide
atggagacagacacaatcctgctatgggtgctgctgctctgggttccaggctccactggtgacattgtgctgacccaatctccagcttctttggctgtgtctctagggcagagggccaccatctcctgcaaggccagccaaagtgttgattatgatggtgatagttatatgaactggttccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatccattcacgttcggctcggggacaaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 62 - DNA, 1403 bp, Artificial Sequence
  Description of Artificial Sequence Synthetic polynucleotide
atggctgtcctggggctgcttctctgcctggtgacgttcccaagctgtgtcctgtcccaggtgcagctgaaggagtcaggacctggcctggtggcaccctcacagagcctgtccatcacatgcactgtctctgggttctcattatccagatatagtgtattttgggttcgccagcctccaggaaagggtctggagtggctgggattgatatggggtggtggaagcacagactataattcagctctcaaatccagactgagcatcagcaaggacaactccaagagccaagttttcttaaaaatgaacagtctgcaaactgatgacacagccatgtactactgtgccagaatctactttgattacgacggggctatggactactggggtcaaggaacctcagtcaccgtctcctcagccaaaacaacgggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctg

SEQ ID NO: 63 - DNA, 732 bp, Artificial Sequence
  Description of Artificial Sequence Synthetic polynucleotide
atgcatcgcaccagcatgggcatcaagatggagtcacggattcaggcatttgtattcgtgtttctctggttgtctggtgttggcggagacattgtgatgacccagtctcacaaattcatgtccacatcagtaggagacagggtcagcgtcacctgcaaggccagtcaggatgtgacttctgctgtagcctggtatcaacaaaaaccagggcaatctcctaaactactgatttactgggcatccacccggcacactggagtccctgatcgcttcacaggcagtggatctgggacagattatactctcaccatcagcagtgggcaggctgaagacctggcactttattactgtcaccaatattatagcgctcctcggacgttcggtggaggcaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 64 - DNA, 1404 bp, Artificial Sequence
  Description of Artificial Sequence Synthetic polynucleotide
atggaatggaactgggtcgttctcttcctcctgtcattaactgcaggtgtctatgcccagggtcagatgcagcagtctggagctgagctggtgaagcctggggcttcagtgaagctgtcctgcaagacttctggcttcaccttcagcagtaactatataagttggttgaagcaaaagcctggacagagtcttgagtggattgcatggatttatgctggaactggtggtattacctataatcagaagttcagaggcagggcccaactgactgtagacacatcctccagcacagcctacatgcagttcagcagcctgacaactgatgactctgccatctattactgtgcaagacacgtgaggggttaccatcctatggactactggggtcaaggaacctcagtcaccgtctcctcagccaaaacgaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 65 - DNA, 705 bp, Artificial Sequence
  Description of Artificial Sequence Synthetic polynucleotide
atggagtcacagactcaggtctttgtatacatgttgctgtggttgtctggtgttgatggagacattgtgatgacccagtctcaaaaattcatgtccgcatcagtaggggacagggtcagcgtcacctgcagggccagtcagaatgtggttactaatgtaggctggtatcaacagaaaccagggcaatctcctaaagtactgatttactcggcatccttccggtacagtggagtccctgatcgcttcacaggcagtggatctgggacagatttcactctcaccatcaccaatgtgcagtctgaagacttggcagagtatttctgtcagcaatataacaactatccgtacacgttcggaggggggaccaagctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 66 - DNA, 1407 bp, Artificial Sequence
  Description of Artificial Sequence Synthetic polynucleotide
atgggatggagctatatcatcctctttttggtagcaacagctacagatgtccactcccaggtccaactgcagcagcctggggctgaactggtgaagcctggggcttcagtgaagctgtcctgcaaggcttctggctacaccttcaccagctactggatgcactgggtgaagcagaggcctggagaaggccttgagtggattggagagattaatcctagctacggtcgtactgactacaatgggaagttcaagaacaaggccacactgactgtagccaaatcctccagcacagcctacatgcaactcagcagcctgacatctgaggactctgcggtctattactgtgcaagaggagattactacggtagtagctcgtttgcttactggggccaagggactctggtcactgtctctgcagccaaaacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagctga

SEQ ID NO: 67 - DNA, 705 bp, Artificial Sequence
  Description of Artificial Sequence Synthetic polynucleotide
atggagtcacagactcaggtctttgtatacatgttgctgtggttgtctggtgttgatggagacattgtgatgacccagtctcaaaaattcatgtccacatcattaggagacagggtcagcgtcacctgcaaggccagtcagaatgtgggtactaatgtagcctggtatcaacagaaaccagggcactctcctaaagcactgatttactcggcatcctaccggtacagtggagtccctgatcgcttcacaggcagtggatctgggacagatttcactctcaccatcagcaatgtgcagtctgaagacttggcagagtttttctgtcagcaatataacaactatccgtacacgttcggaggggggaccacgctcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctatgcctgcgaagtcacccatcagggcctgagctcgcccgtcacaaagagcttcaacaggggagagtgttag

SEQ ID NO: 68 - protein, 466 aa, Artificial Sequence
  Description of Artificial Sequence Synthetic polypeptide
MGRLTSSFLLLIVPAYVLSQVTLKESGPGILQPSQTLSLTCSFSGFSLSTSGMSVGWIRQPSGKGLEWLAHIWWNDDKYYNPVLKSRLTISKETSNNQVFLKIASVVSADTATYYCARFYGNCLDYWGQGTTLTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 69 - protein, 234 aa, Artificial Sequence
  Description of Artificial Sequence Synthetic polypeptide
MMSSAQFLGLLLLCFQGTRCDIQMTQTTSSLSASLGDRVTISCRASQDISNYLNWYQQKPDGTVKLLIYYTSILQLGVPSRFSGSGSETDYSLTISNLEQEDIATYFCQQGDSLPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 464 aa, Artificial Sequence
  Description of Artificial Sequence Synthetic polypeptide
MRALILLCLFTAFPGILSDVQLQESGPDLVKPSQSLSLTCTVTGYSITSGYSWHWIRQFPGNKLEWMGYILFSGSTNYNPSLKSRISITRDTSKNQFFLQLNSVTTEDTATYFCARSNYGSFASWGQGTLVTVSAAKTTGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 71 - protein, 235 aa, Artificial Sequence
  Description of Artificial Sequence Synthetic polypeptide
MDFQVQIFSFLLISASVIISRGQIVLTQSPAIMSASPGEKVTMTCSASSSVSHMHWYQQKSGTSPKRWIYDTSRLASGVPARFSGSGSGTSYSLTISSMEAEDAATYYCQQWSSHPWSFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 468 aa, Artificial Sequence
  Description of Artificial Sequence Synthetic polypeptide
MAWVWTLLFLMAAAQSAQAQIQLVQSGPELKKPGETVKISCKASGYTFTNYGMNWVKQVPGKGLRWMGWMDTFTGEPTYADDFKGRFAFSLETSASTAYLQINSLKNEDTATYFCARGGILRLNYFDYWGQGTTLTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 73 - protein, 234 aa, Artificial Sequence
  Description of Artificial Sequence Synthetic polypeptide
MKFPSQLLLLLLFGIPGMICDIQMTQSSSSFSVSLGDRVTITCKASEDIYNRLGWYQQKPGNAPRLLISGATSLETGVPSRFSGSGSGKDYALSITSLQTEDLATYYCQQCWTSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 464 aa, Artificial Sequence
  Description of Artificial Sequence Synthetic polypeptide
MGWSWIFLFLLSGTGGVLSEVQLQQSGPELVKPGASVKMSCKASGYTFTDYYMKWVKQSHGKSLEWIGDINPNYGDTFYNQKFEGKATLTVDKSSRTAYMQLNSLTSEDSAVYYCGRGDYGYFDVWGAGTTVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLLSLGKAS

SEQ ID NO: 75 - protein, 234 aa, Artificial Sequence
  Description of Artificial Sequence Synthetic polypeptide
METHSQVFVYMLLWLSGVEGDIVMTQSHKFMSTSVGDRVSITCKASQDVGTAVAWYQQKPGQSPKLLIYWASTRHTGVPDRFTGSGSGTDFTLTINNVQSEDLADYFCQQYSSNPYMFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 469 aa, Artificial Sequence
  Description of Artificial Sequence Synthetic polypeptide
MGWSWIFLFLLSGTAGVLSEVQLQQSGPELVKPGASVKISCKTSGYTFTEYTMHWVRSHGKSLEWIGGINPINGGPTYNQKFKGKATLTVDKSSSTAYMELRSLTSEDSAVYYCARWDYGSRDVMDYWGQGTSVTVSSAKTKGPSVFPLAPCSRSTSESTAALGCLVKDYFPEPVPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 77 - protein, 234 aa, Artificial Sequence
  Description of Artificial Sequence Synthetic polypeptide
METHSQVFVYMLLWLSGVEGNIVMTQSPKSMSMSVGERVTLSCKASENVGTYVSWYQQRPEQSPKLLIYGASNRYTGVPDRFTGSGSATDFTLTISSVQAEDLADYHCGQTYSYIFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 467 aa, Artificial Sequence [sequence omitted]

SEQ ID NO: 79 - protein, 234 aa, Artificial Sequence
  Description of Artificial Sequence Synthetic polypeptide
MSVPTQVLGLLLLWLTGARCDIQMAQSPASLSASVGETVTITCRASENIYSYLAWYQQKQGKSPQLLVYNAKTLAEGVPSRFSGSGSGTQFSLKINSLQPEDFGSYYCQHHYGFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 464 aa, Artificial Sequence
  Description of Artificial Sequence Synthetic polypeptide
MNFGLSLIFLVLILKGVQCEVQLVESGGDLVKPGGSLKLSCAASGFTFSSYGMSWVRQTPDKRLEWVAVISSGGSSTFYPDSVKGRFTISRDNAKNTLYLQMSSLKSEDTAVYYCSRGGYYFDYWGQGTTLTVSAAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 81 - protein, 234 aa, Artificial Sequence
  Description of Artificial Sequence Synthetic polypeptide
MRFQVQVLGLLLLWISGAQCDVQITQSPSYLAASPGETITINCRASKTISKYLAWYQEKPEKTDKLLIYSGSTLQSGIPSRFSGSGSGTDFTLTISGLEPEDFAMYYCQQHNEYPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 462 aa, Artificial Sequence
  Description of Artificial Sequence Synthetic polypeptide
MGWSWIFLFLLSGTAGVLSEVQLQQSGPELVKPGASVKISCKASGYSFTGYYMHWVKQSHVKSLEWIGRINPYNGATSYNQNFKDKASLTVDKSSSTAYMELHSLTSEDSAVYYCAREDYVYWGQGTTLTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 83 - protein, 238 aa, Artificial Sequence
  Description of Artificial Sequence Synthetic polypeptide
MKLPVRLLVLMFWIPASSSDVVMTQTPLSLPVSLGDQASISCRSSQSLVHSNGNTYLHWYLQKPGQSPKLLIYKVSNRFSGVPDRFSGSGSGTDFALKISRVEAEDLGVYFCSQSTHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 469 aa, Artificial Sequence
  Description of Artificial Sequence Synthetic polypeptide
MEWSWIFLFLLSGTAGVHSEVQLQQSGPELVKPGASVKMSCKASGYTFTDYVLHWVKQKPGQGLEWIGYINPYNDGTKYNEKFKGKATLTSDKSSSTAYMELSSLTSEDSAVYYCARGYPAYSGYAMDYWGQGTSVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 85 - protein, 234 aa, Artificial Sequence
  Description of Artificial Sequence Synthetic polypeptide
MMSSAQFLGLLLLCFQGTRCDIQMTQTTSSLSASLGDRVTISCRASQDISNYLNWYQQKPDGTVKLLIYYTSRLHSGVPSRFSGSGSGTDYSLTISNLEQEDIATYFCHHGNTLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 467 aa, Artificial Sequence
  Description of Artificial Sequence Synthetic polypeptide
MNLGLSLIFLVLVLKGVQCEVKLVESGGGLVQPGGSLKLSCATSGFTFSDYYMYWVRQTPEKRLEWVAYINSGGGSTYYPDTVKGRFTISRDNAKNTLYLQMSRLKSEDTAMYYCARRGLPFHAMDYWGQGTSVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 87 - protein, 234 aa, Artificial Sequence
  Description of Artificial Sequence Synthetic polypeptide
MMSSAQFLGLLLLCFQGTRCDIQMTQTTSSLSASLGDRVTISCSASQGISNYLNWYQQKPDGTVKLLIYYTSILHSGVPSRFSGSGSGTDYSLTIGNLEPEDIATYYCQQFNKLPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 467 aa, Artificial Sequence
  Description of Artificial Sequence Synthetic polypeptide
MDWLWNLLFLMAAAQSAQAQIQLVQSGPELKKPGETVKISCKASGYSFTNYGMNWVKQAPGKGLKWMGWINTYTGESTYADDFKGRFAFSLETSASTAYLQISNLKNEDMATYFCARGDFRYYYFDYWGQGTTLTGSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 89 - protein, 233 aa, Artificial Sequence
  Description of Artificial Sequence Synthetic polypeptide
MSVLTQVLALLLLWLTGARCDIQMTQSPASLSASVGETVTITCRASGNIHNYLAWYQQKQGKSPQLLVYNAKTLADGVPSRFSGSGSGTQYSLKINTLQPEDFGSYYCQHFWDSWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 465 aa, Artificial Sequence
  Description of Artificial Sequence Synthetic polypeptide
MEWTWVFLFLLSVTAGVHSQVQLQQSGAELMKPGASVKISCKATGYTFSSYWIEWVKQRPGHGLEWIGEILPGSGRTNDNEKFKGKATFTADTSSKKAYMQLSSLTSEDSAVYYCARRGGYSFAYWGQGTLVTVSAAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 91 - protein, 236 aa, Artificial Sequence
  Description of Artificial Sequence Synthetic polypeptide
MTMFSLALLLSLLLLCVSDSRAETTVTQSPASLSMAIGEKVTIRCVTSTDIDDDVNWYQQKPGEPPKLLISEGNTLRPGVPSRFSSSGYGTDFVFTIENMLSEDVADYYCLQSGNLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 467 aa, Artificial Sequence
  Description of Artificial Sequence Synthetic polypeptide
MAWVWTLLFLMAAAQSAQAQIQLVQSGPELKKPGETVKISCKASGYTFTNYGMNWVKQAPGKGLKWVGWINTFTGEPTYVDDFKGRFAFSLETSASTAYLQINNLKNEDTATYFCARGNFRYYYFDYWGQGTTLTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 93 - protein, 234 aa, Artificial Sequence
  Description of Artificial Sequence Synthetic polypeptide
MSVLTQVLALLLLWLTGARCDIQMTQSPASLSASVGETVTITCRTSGNIRNYLAWYQQKQGKSPQLLVYNAKTLADGVPSRFGGSGSGTQYSLKINSLQPEDFGNYYCQHFWSSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 469 aa, Artificial Sequence
  Description of Artificial Sequence Synthetic polypeptide
MMGWSYIILFLVATATDVHSQVQLQQPGAELVKPGASVKLSCKASGYTFTSYWMHWVKQRPGEGLEWIGEINPSYGRTDYNEKFKNKATLTVAKSSSTAYMQLSSLTSEDSAVYYCARGDYYGSSSFAYWGQGTLVTVSAAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 95 - protein, 235 aa, Artificial Sequence
  Description of Artificial Sequence Synthetic polypeptide
MDFQVQIFSFLLMSASVIMSRGQIVLTQSPALMSASPGEKVTMTCSASSNISYMYWYQQKPRSSPKPWIYLTSNLASGVPARFSGSGSGTSYSLTTSSMEAEDAATYCCQQWSSNPPTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 235 aa, Artificial Sequence
  Description of Artificial Sequence Synthetic polypeptide
MDFRVQIFSFLLMSASVIMSRGQIVLTQSPALMSASPGEKVTMTCSASSNISYMYWYQQKPRSSPKPWIYLTSNLASGVPARFSGSGSGTSYSLTISSMEAEDAATYYCQQWSSNPPTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 238 aa, Artificial Sequence
  Description of Artificial Sequence Synthetic polypeptide
METDTLLLWVLLLWVPGSTGDIVLIQSPASLAVSLGQRATISCRASESVDSYVNSFMHWYQQKPGQPPKLLIYRVSNLESGIPARFSGSGSRTDFTLTINPVEADDVATYYCQQSNEDPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 465 aa, Artificial Sequence
  Description of Artificial Sequence Synthetic polypeptide
MECNWILPFILSVISGVYSEVQLQQSGTVLARPGASVNMSCKAAGYSFTSYWVYWVKQRPGQGLEWIGAIYPKNSRTSYNQKFQDKATLTAVTSASTAYMELSSLTNEDSAVYYCTRPHYDSFGYWGQGTLVTVSAAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 99 - protein, 238 aa, Artificial Sequence
  Description of Artificial Sequence Synthetic polypeptide
METDTLLLWVLLLWVPGSTGDIVLTQSPASLAVSLGQRATISCRASESVDSYGISFMHWYQQKPGQPPKLLIYRASNQESGIPARFSGSGSRTDFTLTINPVEADDVATYYCQQSNEDPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 473 aa, Artificial Sequence
  Description of Artificial Sequence Synthetic polypeptide
NRLTSSLLLLIVPAYVLSQQVTLKESGPGILQPSQTLSLTCSFSGFSLSTSGMGVSWIRQPSGKGLEWLAHIYWDDDKRYNPSLKSRLTIFKDPSSNQVFLRITSVDTADTATYYCARNSHYYGSTYGGYFDVWGAGTTVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 101 - protein, 238 aa, Artificial Sequence
  Description of Artificial Sequence Synthetic polypeptide
METDTLLLWVLLLGVPGSTGNIVLTQSPTSFTVSLGQRATISCRASESVHSYGNSFMHWYQQKPGQPPKLLIYLASNVESGVPARFSGSGSRTDFTLTIDPVEADDAATYYCQQNSEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 465 aa, Artificial Sequence
  Description of Artificial Sequence Synthetic polypeptide
MEWTWVFLFLLSVTAGVHSQVQLQQSGTELMKPGASVKISCKATGYTFSTYWIEWVKQRPGHGLEWIGEILPGSGRTNDNEKFKGKATITADTSSKKAYMQLSSLTSEDSAVYYCARRGGYSFAFWGQGTLVSVSAAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 103 - protein, 236 aa, Artificial Sequence
  Description of Artificial Sequence Synthetic polypeptide
MTMFSLALLLSLLLLCVSDSRAETTVTQSPASLSMAIGEKVTIRCVTSTDIDDDVNWYQQKPGEPPKLLISEGNTLRAGVPSRFSSSGYGTDFVFTIENMLSEDVADYYCLQSGNLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 473 aa, Artificial Sequence
  Description of Artificial Sequence Synthetic polypeptide
MNRLTSSLLLLIVPAYVLSQVTLKESGPGILQPSQTLSLTCSFSGFSLSTSGMGLSWIRQPSGKGLEWLAHIYWDDDKRYNPSLKSRLTISKDTSSNQVFLKITIVDTADAATYYCARSSHYYGYGYGGYFDVWGAGTTVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 105 - protein, 238 aa, Artificial Sequence
  Description of Artificial Sequence Synthetic polypeptide
METDTLLLWVLLLWVPGSTGNIVLTQSPASLAVSLGQRATISCRASESIHSYGNSFLHWYQQKPGQPPKLLIYLASNLESGVPARFSGSGSRTDFTLTIDPVEADDAATYYCQQNNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 467 aa, Artificial Sequence
  Description of Artificial Sequence Synthetic polypeptide
MKCSWVIFFLMAVVTGVNSEVQLQQSGAELVRPGALVKLSCKASGFNINDYYIHWVKQRPEQGLERIGWIDPDNGNTIYDPKFQGKASITADTSPNTAYLQLSSLTSEDTAVYYCARTRSPMVTTGFVYWGQGTVVTVSAAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKXKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 107 - protein, 474 aa, Artificial Sequence
  Description of Artificial Sequence Synthetic polypeptide
MERHWIFLFLFSVTAGVHSQVQLQQSGAELAKPGASVKMSCKASGYTFTTYWMHWVKQRPGQGLEWIGYINPITGYTEYNQKFKDKATLTADKSSSTAYMQLSSLTSEDSAVYYCAREGLSAMDYWGQGTSVTVTSAKTTAPSVYPLAPVCGDTTGSSVTLGCLVKGYFPEPVTLTWNSGSLSSGVHTFPAVLQSDLYTLSSSVTVTSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGKAS

SEQ ID NO: 108 - protein, 234 aa, Artificial Sequence
  Description of Artificial Sequence Synthetic polypeptide
MESRIQAFVFVFLWLSGVGGDIVMTQSHKFMSTSVGDRVSVTCKASQDVTSAVAWYQQKPGQSPKLLIYWASTRHTGVPDRFTGSGSGTDYTLTISSGQAEDLALYYCHQYYSAPRTFGGGTKLEVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 109 - protein, 465 aa, Artificial Sequence
  Description of Artificial Sequence Synthetic polypeptide
MERHWIFLFLFSVTAGVHSQVQLQQSGAELAKPGASVKMSCKASGYTFTTYWMHWVKQRPGQGLEWIGYINPITGYTEYNQKFKDKATLTADKSSSTAYMQLSSLTSEDSAVYYCAREGLSAMDYWGQGTSVTVTSAKTTGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 110 - protein, 234 aa, Artificial Sequence
  Description of Artificial Sequence Synthetic polypeptide
MESQIQAFVFVFLWLSGVGGDIVMTQSHKFMSTSVGDRVSVTCKASQDVTSAVAWYQQKPGQSPKLLIYWASTRHTGVPDRFTGSGSGTDYTLTISSGQAEDLALYYCHQYYSAPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 466 aa, Artificial Sequence
  Description of Artificial Sequence Synthetic polypeptide
MAVLALLLCLVAFPTCTLSQVQLKESGPGLVAPSQSLSITCSVSGFSLSNYDISWIRQPPGKGLEWLGVMWTGGGANYNSAFMSRLSINKDNSKSQVFLKMNNLQTDDTAIYYCVRDAVRYWNFDVWGAGTTVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 112 - protein, 235 aa, Artificial Sequence
  Description of Artificial Sequence Synthetic polypeptide
MDFQAQIFSFLLISASVIMSRGQIVLSQSPAILSASPGEKVTMTCRASSSVSYIHWYQQKPGSSPKPWIYATSHLASGVPARFSGSGSGTSYSLTISRVEAEDTATYYCQQWSSNPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 469 aa, Artificial Sequence
  Description of Artificial Sequence Synthetic polypeptide
MERHWIFLLLLSVTAGVHSQVQLQQSGAELARPGASVKMSCKASGYTFTTYTMHWVKQRPGQGLEWIGYINPSSGYTNYNQKFKDKATLTADKSSSTASMQLSSLTSEDSAVYYCARERAVLVPYAMDYWGQGTSVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 114 - protein, 235 aa, Artificial Sequence
  Description of Artificial Sequence Synthetic polypeptide
MHFQVQIFSFLLISASVIMSRGQIVLTQSPAVMSASPGEKVTITCTASSSLSYMHWFQQKPGTSPKLWLYSTSILASGVPTRFSGSGSGTSYSLTISRMEAEDAATYYCQQRSSSPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 467 aa, Artificial Sequence
  Description of Artificial Sequence Synthetic polypeptide
MGWTWIFILILSVTTGVHSEVQLQQSGPELEKPGASVKISCKASGYSFTGYNMNWVKQSNGKSLEWIGNIDPYYGDTNYNQKFKGKATLTVDKSSSTAYMHLKSLTSEDSAVYYCARPYGSEAYFAYWGQGTLVTVSAAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 116 - protein, 214 aa, Artificial Sequence
  Description of Artificial Sequence Synthetic polypeptide
DIVMTQSPATLSVTPGDRVSLSCRASQSISDYLHWYQQKSHESPRLLIKYAAQSISGIPSRFSGSGSGSDFTLSINGVEPEDVGVYYCQNGHSFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 446 aa, Artificial Sequence
  Description of Artificial Sequence Synthetic polypeptide
QVQLRQSGPELVKPGASVKMSCKASGYTFTDYVISWVKQRTGQGLEWIGDIYPGSGYSFYNENFKGKATLTADKSSTTAYMQLSSLTSEDSAVYFCATYYNYPFAYWGQGTLVTVSAAKTTGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 118 - protein, 219 aa, Artificial Sequence
  Description of Artificial Sequence Synthetic polypeptide
DVVMTQTPLSLPVRLGDQASISCRSSQSLVHSNGNTYLHWYLQKPGQSPKLLIYKVSNRFSGVPDRFSGSGSGTNFTLKISRVEAEDLGLYFCSQSTHVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 469 aa, Artificial Sequence
  Description of Artificial Sequence Synthetic polypeptide
MTLNMLLGLRWVFFVVFYQGVHCEVQLVESGGGLVQPKGSLKLSCAASGLTFNIYAMNWVRQAPGKGLEWVARIRNKSNNYATYYADSVKDRFTISRDDSQSLLYLQMNNLKTEDTAMYYCVGRDWFDYWGQGTLVTVSAAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 120 - protein, 235 aa, Artificial Sequence
  Description of Artificial Sequence Synthetic polypeptide
MAWISLILSLLALSSGAISQAVVTQESALTTSPGETVTLTCRSSTGAVTTSNYANWVQEKPDHLFTGLIGGTNNRVSGVPARFSGSLIGDKAALTITGAQTEDEAIYFCALWYSNHWVFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 461 aa, Artificial Sequence
  Description of Artificial Sequence Synthetic polypeptide
MEWTWVFLFLLSVTAGVHSQVQLQQSGAELMKPGASVKISCKATGYTFGSYWIEWVKQRPGHGLEWIGEILPGSGNTNYNENFKGKATFTADTSSNTAYMQLTSLTSEDSAVYYCARAGIYWGQGTLVTVSAAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 122 - protein, 238 aa, Artificial Sequence
  Description of Artificial Sequence Synthetic polypeptide
MEKDTLLLWVLLLWVPGSTGDIVLTQSPAFLAVSLGQRATISCRASESVDNYGISFMNWFQQKPGQPPKLLIYVASKQGSGVPARFSGSGSGTDFSLNIHPMEEDDTAMYFCQQSKEVPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - protein, 463 aa, Artificial Sequence
  Description of Artificial Sequence Synthetic polypeptide
MECNWILPFILSVTSGVYSEVQLQQSGTVLARPGASVKMSCKASGYTFTSYWMHWVKQRPGQGLEWIGAIYPGNSDTTYNQKFKGKAKLTAVTSTSTAYMELSSLTNEDSAVYYCTPTYYFDYWGQGTSLTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 124 - protein, 238 aa, Artificial Sequence
  Description of Artificial Sequence Synthetic polypeptide
MSPAQFLFLLVLWIRETNGDVVMTQTPLTLSVTIGQPASISCKSSQSLLDSDGKTYLNWFLQRPGQSPKRLIYLVSKLDSGVPDRFTGSGSGTDFTLKISRVEAEDLGVYYCWQGTHFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 468 aa, Artificial Sequence
  Description of Artificial Sequence Synthetic polypeptide
MGGIWIFLFLLSGTAGAHSEIQLQQTGPELVKPGASVKISCKASGYPFTDYIMVWVKQSHGKSLEWIGNISPYYGTTNYNLKFKGKATLTVDKSSSTAYMQLNSLTSEDSAVYYCARSPNWDGAWFAHWGQGALVTVSAAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 126 - protein, 238 aa, Artificial Sequence
  Description of Artificial Sequence Synthetic polypeptide
METDTILLWVLLLWVPGSTGDIVLTQSPASLAVSLGQRATISCKASQSVDYDGDSYMNWFQQKPGQPPKLLIYAASNLESGIPARFSGSGSGTDFTLNIHPVEEEDAATYYCQQSNEDPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 467 aa, Artificial Sequence
  Description of Artificial Sequence Synthetic polypeptide
MAVLGLLLCLVTFPSCVLSQVQLKESGPGLVAPSQSLSITCTVSGFSLSRYSVFWVRQPPGKGLEWLGLIWGGGSTDYNSALKSRLSISKDNSKSQVFLKMNSLQTDDTAMYYCARIYFDYDGAMDYWGQGTSVTVSSAKTTGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 128 - protein, 243 aa, Artificial Sequence
  Description of Artificial Sequence Synthetic polypeptide
MHRTSMGIKMESRIQAFVFVFLWLSGVGGDIVMTQSHKFMSTSVGDRVSVTCKASQDVTSAVAWYQQKPGQSPKLLIYWASTRHTGVPDRFTGSGSGTDYTLTISSGQAEDLALYYCHQYYSAPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 467 aa, Artificial Sequence
  Description of Artificial Sequence Synthetic polypeptide
MEWNWVVLFLLSLTAGVYAQGQMQQSGAELVKPGASVKLSCKTSGFTFSSNYISWLKQKPGQSLEWIAWIYAGTGGITYNQKFRGRAQLTVDTSSSTAYMQFSSLTTDDSAIYYCARHVRGYHPMDYWGQGTSVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 130 - protein, 234 aa, Artificial Sequence
  Description of Artificial Sequence Synthetic polypeptide
MESQTQVFVYMLLWLSGVDGDIVMTQSQKFMSASVGDRVSVTCRASQNVVTNVGWYQQKPGQSPKVLIYSASFRYSGVPDRFTGSGSGTDFTLTITNVQSEDLAEYFCQQYNNYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 468 aa, Artificial Sequence
  Description of Artificial Sequence Synthetic polypeptide
MGWSYIILFLVATATDVHSQVQLQQPGAELVKPGASVKLSCKASGYTFTSYWMHWVKQRPGEGLEWIGEINPSYGRTDYNGKFKNKATLTVAKSSSTAYMQLSSLTSEDSAVYYCARGDYYGSSSFAYWGQGTLVTVSAAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 132 - protein, 234 aa, Artificial Sequence
  Description of Artificial Sequence Synthetic polypeptide
MESQTQVFVYMLLWLSGVDGDIVMTQSQKFMSTSLGDRVSVTCKASQNVGTNVAWYQQKPGHSPKALIYSASYRYSGVPDRFTGSGSGTDFTLTISNVQSEDLAEFFCQQYNNYPYTFGGGTTLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 60 aa, Artificial Sequence
  Description of Artificial Sequence Synthetic polypeptide
MWVPVVFLTLSVTWIGAAPLILSRIVGGWECEKHSQPWQVLVASRGRAVCGGVLVHPQWV

SEQ ID NO: 134 - protein, 60 aa, Artificial Sequence
  Description of Artificial Sequence Synthetic polypeptide
LTAAHCIRNKSVILLGRHSLFHPEDTGQVFQVSHSFPHPLYDMSLLKNRFLRPGDDSSHD

SEQ ID NO: 135 - protein, 60 aa, Artificial Sequence
  Description of Artificial Sequence Synthetic polypeptide
LMLLRLSEPAELTDAVKVMDLPTQEPALGTTCYASGWGSIEPEEFLTPKKLQCVDLHVIS

SEQ ID NO: 136 - protein, 60 aa, Artificial Sequence
  Description of Artificial Sequence Synthetic polypeptide
NDVCAQVHPQKVTKFMLCAGRWTGGKSTCSGDSGGPLVCNGVLQGITSWGSEPCALPERP

SEQ ID NO: 137 - protein, 21 aa, Artificial Sequence
  Description of Artificial Sequence Synthetic peptide
SLYTKVVHYRKWIKDTIVANP

SEQ ID NO: 138 - protein, 9 aa, Artificial Sequence
  Description of Artificial Sequence Synthetic peptide
IMDQVPFSV

SEQ ID NO: 139 - protein, 9 aa, Artificial Sequence
  Description of Artificial Sequence Synthetic peptide
ITDQVPFSV

SEQ ID NO: 140 - protein, 9 aa, Artificial Sequence
  Description of Artificial Sequence Synthetic peptide
YLEPGPVTV

SEQ ID NO: 141 - protein, 9 aa, Artificial Sequence
  Description of Artificial Sequence Synthetic peptide
YLEPGPVTA

SEQ ID NO: 142 - protein, 9 aa, Artificial Sequence
  Description of Artificial Sequence Synthetic peptide
KTWGQYWQV

SEQ ID NO: 143 - protein, 230 aa, Artificial Sequence
  Description of Artificial Sequence Synthetic polypeptide
DTTEPATPTTPVTTPTTTKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSQSYVPLAHSSSAFTITDQVPFSVSVSQLRALDGGNKHFLRNQ

SEQ ID NO: 144 - protein, 73 aa, Artificial Sequence
  Description of Artificial Sequence Synthetic polypeptide
PLTFALQLHDPSGYLAEADLSYTWDFGDSSGTLISRAXVVTHTYLEPGPVTAQVVLQAAIPLTSCGSSPVPAS

SEQ ID NO: 145 - protein, 109 aa, Artificial Sequence
  Description of Artificial Sequence Synthetic polypeptide
GTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAA

SEQ ID NO: 146 - protein, 75 aa, Artificial Sequence
  Description of Artificial Sequence Synthetic polypeptide
QVTTTEWVETTARELPIPEPEGPDASSIMSTESITGSLGPLLDGTATLRLVKRQVPLDCVLYRYGSFSVTLDIVQ

SEQ ID NO: 147 - protein, 109 aa, Artificial Sequence
  Description of Artificial Sequence Synthetic polypeptide
GIESAEILQAVPSGEGDAFELTVSCQGGLPKEACMEISSPGCQPPAQRLCQPVLPSPACQLVLHQILKGGSGTYCLNVSLADTNSLAVVSTQLIVPGILLTGQEAGLGQ

SEQ ID NO: 148 - protein, 50 aa, Artificial Sequence
  Description of Artificial Sequence Synthetic polypeptide
MEMKILRALNFGLGRPLPLHFLRRASKIGEVDVEQHTLAKYLMELTMLDY

SEQ ID NO: 149 - protein, 36 aa, Artificial Sequence
  Description of Artificial Sequence Synthetic polypeptide
DWLVQVQMKFRLLQETMYMTVSIIDRFMQNNCVPKK

SEQ ID NO: 150 - protein, 48 aa, Artificial Sequence
  Description of Artificial Sequence Synthetic polypeptide
MEHQLLCCEVETIRRAYPDANLLNDRVLRAMLKAEETCAPSVSYFKCV

SEQ ID NO: 151 - protein, 95 aa, Artificial Sequence
  Description of Artificial Sequence Synthetic polypeptide
QKEVLPSMRKIVATWMLEVCEEQKCEEEVFPLAMNYLDRFLSLEPVKKSRLQLLGATCMFVASKMKETIPLTAEKLCIYTDNSIRPEELLQMELL

SEQ ID NO: 152 - protein, 60 aa, Artificial Sequence
  Description of Artificial Sequence Synthetic polypeptide
LVNKLKWNLAAMTPHDFIEHFLSKMPEAEENKQIIRKHAQTFVALCATDVKFISNPPSMV

SEQ ID NO: 153 - protein, 91 aa, Artificial Sequence
  Description of Artificial Sequence Synthetic polypeptide
AGSVVAAVQGLNLRSPNNFLSYYRLTRFLSRVIKCDPDCLRACQEQIEALLESSLRQAQQNMDPKAAEEEEEEEEEVDLACTPTDVRDVDI

SEQ ID NO: 154 - protein, 32 aa, Human immunodeficiency virus 1
VGFPVTPQVPLRPMTYKAAVDLSHFLKEKGGL

SEQ ID NO: 155 - protein, 30 aa, Human immunodeficiency virus 1
HTQGYFPDWQNYTPGPGVRYPLTFGWLYKL

SEQ ID NO: 156 - protein, 19 aa, Human immunodeficiency virus 1
EKIRLRPGGKKKYKLKHIV

SEQ ID NO: 157 - protein, 32 aa, Human immunodeficiency virus 1
NPPIPVGEIYKRWIILGLNKIVRMYSPTSILD

SEQ ID NO: 158 - protein, 31 aa, Human immunodeficiency virus 1
AIFQSSMTKILEPFRKQNPDIVIYQYMDDLY

SEQ ID NO: 159 - protein, 9 aa, Unknown
  Description of Unknown prostate specific antigen peptide
KLQCVDLHV

SEQ ID NO: 160 - protein, 9 aa, Influenza A virus
GILGFVFTL